(19) United States
(12) Patent Application Publication
(10) Pub. No.: US 2009/0054450 A1
(43) Pub. Date: Feb. 26, 2009

(54) COMPOSITIONS AND METHODS OF USE FOR TREATING OR PREVENTING LIPID RELATED DISORDERS

(75) Inventors: Mark G. CURRIE, Sterling, MA (US); John Jeffrey TALLEY, Somerville, MA (US); Brian CALI, Arlington, MA (US)

Correspondence Address: HESLIN ROTHENBERG FARLEY & MESITI PC, 5 COLUMBIA CIRCLE, ALBANY, NY, 12203, US

(73) Assignee: IRONWOOD PHARMACEUTICALS, INC., Cambridge, MA (US)

(21) Appl. No.: 12/140,637

(22) Filed: Jun. 17, 2008

Related U.S. Application Data
(60) Provisional application No. 60/944,934, filed on Jun. 19, 2007.
(60) Provisional application No. 61/023,744, filed on Jan. 25, 2008.
(60) Provisional application No. 61/030,778, filed on Feb. 22, 2008.

(51) Int. Cl. A61K 31/506; C07C 69/00; A61K 31/216; C07C 233/01; A61K 31/164; C07D 257/04; A61K 31/41; C07C 229/02; A61P 3/00; A61K 31/192; C07D 263/02; A61K 31/421; C07D 403/02; A61K 31/496; C07D 403/14

(52) U.S. Cl. 514/252.19; 560/52; 514/534; 564/169; 514/621; 548/251; 514/381; 564/147; 514/615; 562/433; 514/563; 548/233; 514/377; 544/370; 514/254.05; 544/295

(57) ABSTRACT

Disclosed herein are novel compositions and methods for treating or preventing a variety of disorders and conditions associated with lipid metabolism. The methods generally include administering to a patient in need thereof a therapeutically effective amount of a pharmaceutical composition comprising one or more fibric acid or statin derivative compositions alone or in combination with one or more lipid altering agents and/or PDE inhibitors.

### CROSS-REFERENCE TO RELATED APPLICATIONS

This application claims benefit from U.S. Provisional Application Nos. 60/944,934 filed Jun. 19, 2007; 61/023,744 filed Jan. 25, 2008; and 61/030,778 filed Feb. 22, 2008, the entire contents of which are incorporated herein by reference.

### TECHNICAL FIELD

The subject matter of this application relates to fibric acid and statin derivatives, and pharmaceutical formulations thereof, used alone or in combination with one or more additional agents for treating lipid related disorders and associated conditions.

### BACKGROUND

Disorders of lipid metabolism or dyslipidemias include various conditions characterized by abnormal concentrations of one or more lipids (i.e., cholesterol and triglycerides), and/or apolipoproteins (i.e., apolipoproteins A, B, C and E), and/or lipoproteins (i.e., the macromolecular complexes formed by the lipid and the apolipoprotein that allow lipids to circulate in blood, such as LDL, VLDL and IDL). Dyslipidemia is a major risk factor for cardiovascular disorders including coronary heart disease. Dyslipidemias were originally classified by Fredrickson according to the combination of alterations mentioned above. The Fredrickson classification includes 6 phenotypes (i.e., I, IIa, IIb, III, IV and V) with the most common being the isolated hypercholesterolemia (or type IIa), which is usually accompanied by elevated concentrations of total and LDL cholesterol. A second common form of dyslipidemia is the mixed or combined hyperlipidemia or type IIb and III of the Fredrickson classification. This dyslipidemia is often prevalent in patients with type 2 diabetes, obesity and the metabolic syndrome.

The management of dyslipidemia is an important part of the National Cholesterol Education Program Adult Treatment Panel III guidelines. The reduction of low-density lipoprotein cholesterol as the primary target of treatment is recommended. Various lipid altering agents are considered first-line drugs for attaining this goal. However, monotherapy may not always be optimal for patients with significant combined or mixed dyslipidemias (e.g., elevated low-density lipoprotein cholesterol plus hypertriglyceridemia) or with concomitant conditions that increase the patient's level of risk (e.g., type 2 diabetes or the metabolic syndrome). Accordingly, combination drug therapy, which utilizes complementary mechanisms of action to alter levels of one or more lipoproteins may be useful.

It would be desirable to develop an effective treatment for management of lipid related disorders using fibric acid and statin derivatives that can be administered alone or in combination with one or more lipid altering agents. Lipid altering agents encompass several classes of drugs that include HMG CoA reductase inhibitors (statins), fibric acid derivatives (fibrates), cholesterol-ester-transfer-protein (“CETP”) inhibitors, squalene synthase inhibitors, microsomal-triglyceride-transfer-protein (“MTTP”) inhibitors, cholesterol absorption inhibitors (“CAIs”), soluble guanylate cyclase modulators (“sGC modulators”), bile acid sequestrants, nicotinic acid, thyroid receptor agonists, liver X-receptor (LXR) modulators, antisense inhibitors of apoB-100 or C reactive protein, and probucol and derivatives thereof (e.g. AGI-1067). These drugs differ with respect to mechanism of action and to the degree and type of lipid modulation. Thus, the indications for a particular drug are influenced by the underlying lipid abnormality. Monotherapy with statins, which competitively inhibit the intracellular rate-limiting enzyme for cholesterol biosynthesis, and bile acid sequestrants, which reduce terminal ileal bile acid absorption, primarily lower plasma low-density lipoprotein (LDL) cholesterol by enhancing hepatic LDL-receptor activity. Monotherapy with fibrates, which serve as ligands for peroxisome proliferator-activated receptor α, a transcription factor influencing gene expression in lipid metabolism, reduces plasma very-low-density lipoprotein (VLDL) cholesterol and triglycerides, and also increases high-density lipoprotein (HDL) cholesterol.

### SUMMARY

Briefly, the present application discloses compositions of fibric acid and statin derivatives and their use in methods of prevention and/or treatment of various lipid related disorders, wherein the administration of such compositions is to a subject in need thereof. The application also discloses pharmaceutical formulations comprising fibric acid and statin derivatives alone or in combination with one or more lipid altering agents.

In accordance with the above, the present application discloses methods to prevent and/or treat lipid related disorders such as dyslipidemia, hypercholesterolemia, hypertriglyceridemia, sitosterolemia, and fatty liver disease, by administering a therapeutically effective dose of at least one fibric acid or statin derivative described herein, alone or in combination with another therapeutic agent such as a lipid altering agent or a PDE inhibitor.

In a first aspect, compounds represented by the structure of Formula I or II:

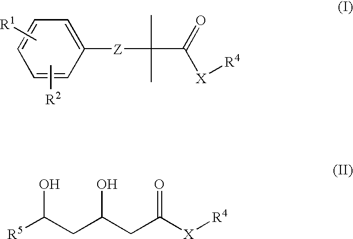

wherein

R¹ is chosen from H and halogen;

R² is chosen from H, halogen, cycloalkyl substituted with from 1 to 3 halogens, COR³, and (CH₂)ₘNHOR³;

R³ is phenyl substituted with from one to three halogen groups;

Z is chosen from O and (CH₂)ₙO;

X is chosen from direct bond, O, NH, and an amino acid residue;

R⁴ is chosen from OH, NO, NO₂, an amino acid residue, a fibric acid residue, guanidine, tetrazolyl, agmatine, an amino-containing compound; lower alkyl terminating in ONO, (ONO₂)ₚ, or guanidine; a resveratrol residue; and an imidazoline receptor agonist residue;

wherein m, n, and p are independently chosen from 1 to 3; and

R⁵ is chosen from a residue of a statin

are provided herein.

In certain embodiments, the compositions can include therapeutically effective amounts of the compounds represented by Formula (I), which include, but are not limited to, any of the compounds for which the synthesis is shown in Examples 1-35. In other embodiments, the compositions can include therapeutically effective amounts of the compounds represented by Formula (II), which include, but are not limited to, any of the compounds for which the synthesis is shown in Examples 36-115. In still other embodiments, the present disclosure provides compounds comprising a fibric acid or statin derivative in the form of a salt, wherein a fibric acid or statin residue is a cation or anion, and another molecule is presented as a counterion to the fibric acid or statin residue. In some embodiments, the counterion includes, but is not limited to, a NOS substrate or an amino-tetrazole compound. Synthesis of particular fibric acid or statin derivative salt compounds is shown in Examples 116-139, although any fibric acid or statin compound can be used. Similarly, molecules other than those disclosed in the instant Examples can be used as the corresponding counterions.

In other embodiments, the instant disclosure provides compositions that include one or more fibric acid or statin compound and one or more amino-containing compound. Although the fibric acid compound can include any fibric acid, in certain embodiments the fibric acid is fenofibrate (CA Registry No. 49562-28-9). Similarly, although any statin can be used, in certain embodiments the statin includes, but is not limited to, atorvastatin, rosuvastatin, simvastatin, and fluvastatin. In other embodiments, the amino-containing compound includes, but is not limited to, agmatine, aminoguanidine, guanidine, tetrazole, amino-tetrazole, or an amino acid residue. While any amino acid residue can be used, in certain embodiments, the amino acid is alanine, lysine, or arginine.

In certain embodiments, the fibric acid or statin compound is formulated and administered in combination with the amino-containing compound. In other embodiments, the fibric acid or statin compound and amino-containing compound are formulated and administered separately. In certain embodiments, one or more additional agents for treating lipid related disorders and associated conditions can also be co-formulated or co-administered.

In other embodiments, the instant disclosure provides compositions that include one or more fibric acid or statin compound and resveratrol. Although the fibric acid compound can include any fibric acid, in certain embodiments the fibric acid is fenofibrate. Similarly, although any statin can be used, in certain embodiments the statin compound includes, but is not limited to, atorvastatin, rosuvastatin, simvastatin, and fluvastatin.

In certain embodiments, the fibric acid or statin compound is formulated and administered in combination with resveratrol. In other embodiments, the fibric acid or statin compound and resveratrol compound are formulated and administered separately. In certain embodiments, one or more additional agents for treating lipid related disorders and associated conditions can also be co-formulated or co-administered.

In other embodiments, the instant disclosure provides compositions that include one or more fibric acid or statin compound and one or more imidazo line receptor agonists. Although the fibric acid compound can include any fibric acid, in certain embodiments the fibric acid is fenofibrate. Similarly, although any statin can be used, in certain embodiments the statin compound includes, but is not limited to, atorvastatin, rosuvastatin, simvastatin, and fluvastatin. In some embodiments, the imidazoline receptor agonist includes, but is not limited to, LNP509, S-21663, S-22068 or S-23515. In some embodiments, the imidazoline receptor agonist does not agonize one or more adrenergic receptors (e.g. α2-adrenergic receptors). In other embodiments, the imidazoline receptor agonist agonizes one or more adrenergic receptors (e.g. α2-adrenergic receptors). In some embodiments the imidazoline receptor agonist is selective for the I1 imidazoline receptor (e.g. and is not an agonist of either the I2 or I3 imidazoline receptor). In some embodiments the imidazoline receptor agonist does not cross the blood brain barrier.

In certain embodiments, the fibric acid or statin compounds are formulated and administered in combination with one or more imidazoline receptor agonists. In other embodiments, they are formulated and administered separately. In certain embodiments, one or more additional agents for treating lipid related disorders and associated conditions can also be co-formulated or co-administered.

In other embodiments, the compositions disclosed herein can include a therapeutically effective amount of at least one fibric acid or statin derivative compound or composition disclosed herein and a therapeutically effective amount of at least one lipid altering agent and/or at least one phosphodiesterase inhibitor. Thus, in certain embodiments the therapeutically effective amount of at least one fibric acid or statin derivative compound or composition can be co-administered, either simultaneously or sequentially, with a therapeutically effective amount of at least one lipid altering agent and/or at least one phosphodiesterase inhibitor.

In certain embodiments, the lipid altering agent can include, for example, statins, fibrates, cholesterol-ester-transfer-protein (CETP) inhibitors, squalene synthase inhibitors, microsomal-triglyceride-transfer-protein (MTTP) inhibitors, cholesterol absorption inhibitors, soluble guanylate cyclase modulators, and bile acid sequestrants. Other suitable compounds are also disclosed herein. Suitable inorganic cholesterol sequestrants include bismuth salicylate plus montmorillonite clay, aluminum hydroxide and calcium carbonate antacids.

In certain embodiments, the lipid altering agent includes a fibrate, such as, for example, fenofibrate.

In other embodiments, the lipid altering agent includes a statin. In certain embodiments, the statin includes, but is not limited to, atorvastatin (Lipitor®), bervastatin, carvastatin, crilvastatin, dalvastatin, fluvastatin (Lescol®), glenvastatin, fluindostatin, velostatin, lovastatin (mevinolin; Mevacor®), pravastatin (Pravachol®), rosuvastatin (Crestor®), and simvastatin (Zocor®).

In still other embodiments, the lipid altering agent includes a soluble guanylate cyclase modulator (sGC modulator). The usefulness of such compounds in treating lipid related disorders is disclosed in U.S. Provisional Application No. 60/910,309 filed Apr. 5, 2007, which is incorporated herein by reference.

In certain embodiments, the sGC modulator can include, for example, one or more of the following compounds: nitroglycerin, isosorbide dinitrate, isosorbide mononitrate, isosorbide 5-mononitrate, sodium nitroprusside, FK 409 (NOR-3); FR 144420 (NOR-4); 3-morpholinosydnonimine; Linsidomine chlorohydrate (“SIN-1”); S-nitroso-N-acetylpenicillamine (“SNAP”); AZD3582 (ClNOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), S-nitrosoglutathione (GSNO), S-nitrosoglutathione mono-ethyl-ester (GSNO-ester), 6-(2-hydroxy-1-methyl-nitrosohydrazino)-N-methyl-1-hexanamine (NOC-9) or diethylamine NONOate, S-nitrosothiol, a nitrite, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea or a furoxan, including pharmaceutically acceptable salts or mixtures thereof.

In certain embodiments, the one or more sGC modulators are chosen from NO donors, eNOS transcriptional enhancers, haem-dependent sGC stimulators, haem-independent sGC activators and NOS substrates. In certain embodiments, the NO donor is chosen from organic nitrates, isosorbides, S-nitrosothiols, iron-nitrosyl complexes, sydnonimines, C-nitroso compounds, and secondary amine/NO complex ions. In certain embodiments, the organic nitrates can include, for example nitroglycerin and isosorbides. Isosorbides include, but are not limited to, isosorbide dinitrate and isosorbide mononitrate. In certain embodiments, the isosorbide dinitrate can include, for example, dilatrate SR.

In other embodiments, the soluble guanylate cyclase modulator is an eNOS transcriptional enhancer. In certain embodiments, the eNOS transcriptional enhancer includes, but is not limited to, 2,2-difluorobenzo[1,3]dioxol-5-carboxylic acid indan-2-ylamide, 4-fluoro-N-(indan-2-yl)-benzamide), AVE3085 and AVE9488.

In other embodiments, the soluble guanylate cyclase modulator is haem-dependent sGC stimulator. In certain embodiments, the haem-dependent sGC stimulator includes, but is not limited to, YC-1, BAY 41-2272, BAY 41-8543, CFM-1571, and A350-619.

In other embodiments, the soluble guanylate cyclase modulator is haem-independent sGC activator. In certain embodiments, the haem-independent sGC activator includes, but is not limited to, BAY 58-2667, HMR-1766, S 3448 (2-(4-chloro-phenylsulfonylamino)-4,5-dimethoxy-N-(4-(thiomorpholine-4-sulfonyl)-phenyl)-benzamide and HMR-1069.

In other embodiments, the soluble guanylate cyclase modulator is a NOS substrate. In certain embodiments, the NOS substrate includes, but is not limited to, arginine, an n-hydroxyguanidine based analog, an L-arginine derivative, an N-alkyl-N′-hydroxyguanidine, an N-aryl-N′-hydroxyguanidine and a guanidine derivatives. In other embodiments, the NOS substrate includes, but is not limited to, N[G]-hydroxy-L-arginine (NOHA), (1-(3,4-dimethoxy-2-chlorobenzylideneamino)-3-hydroxyguanidine), PR5 (1-(3,4-dimethoxy-2-chlorobenzylideneamino)-3-hydroxyguanidine), homo-Arg, homo-NOHA, N-tert-butyloxy-(3-methyl-2-butenyl)oxy-L-arginine, N-(3-methyl-2-butenyl)oxy-L-arginine, canavanine, epsilon guanidine-caproic acid, agmatine, hydroxyl-agmatine, L-tyrosyl-L-arginine), N-cyclopropyl-N′-hydroxyguanidine, N-butyl-N′-hydroxyguanidine, N-phenyl-N′-hydroxyguanidine, a para-substituted derivative of N-phenyl-N′-hydroxyguanidine and 3-(trifluormethyl) propylguanidine.

In other embodiments, the lipid altering agent includes a bile acid sequestrant. In certain embodiments, the bile acid sequestrant includes, but is not limited to, cholestyramine, colesevelam, sevelamer, and colestipol.

In other embodiments, the lipid altering agent includes a cholesterol absorption inhibitor (CAI). In certain embodiments, the CAI includes, but is not limited to, 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; 4-biphenylylazetidinones. In other embodiments, the CAI includes, but is not limited to, (1S)-1,5-Anhydro-1-(4′-{(2S,3R)-3-[(3S)-3-(4-fluorophenyl)-3-hydroxypropyl]-4-oxo-1-phenylazetidin-2-yl}-3′-hydroxybiphenyl-4-yl)-D-glucitol, (3R,4S)-4-(3,3′-Dihydroxybiphenyl-4-yl)-3-[(3S)-3-(4-fluorophenyl)-3-hydroxypropyl]-1-phenylazetidin-2-one, (4′-{(2S,3R)-3-[(3S)-3-(4-Fluorophenyl)-3-hydroxypropyl]-4-oxo-1-phenylazetidin-2-yl}-3′-hydroxybiphenyl-4-yl)phosphonic acid, AVE5530 (Aventis), and AZD-4121 (Aztrazeneca). In some embodiments, the CAI can be ezetimibe.

In certain embodiments, the phosphodiesterase inhibitor can include, for example, PDE3, PDE4, or PDE5. In some embodiments, the PDE5 inhibitor is chosen from sildenafil, tadalafil, vardenafil, udenafil and avanafil, or any other inhibitor of an enzyme that accepts cGMP and breaks it down.

In a second aspect, methods for treating or preventing a lipid metabolism disorder by administering to a patient in need thereof a therapeutically effective amount of at least one fibric acid or statin derivative compound or composition disclosed herein, alone or in combination with a therapeutically effective amount of at least one lipid altering agent and/or at least one phosphodiesterase inhibitor, are provided.

In certain embodiments, the patient may be suffering from (or susceptible to developing) a lipid metabolism disorder including, but not limited to, dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, and fatty liver disease. In certain embodiments, the hypercholesterolemia includes, for example, primary heterozygous familial hypercholesterolemia or primary non-familial hypercholesterolemia.

In certain embodiments, the pharmaceutical composition is in a form suitable for oral administration. In other embodiments the fibric acid or statin compound or pharmaceutical formulation is administered simultaneously with the lipid altering agent and/or PDE inhibitor. In yet other embodiments the fibric acid or statin compound or pharmaceutical formulation is administered sequentially to the lipid altering agent and/or PDE inhibitor.

In yet another aspect, kits for treating a lipid metabolism disorder or associated condition comprising, in one or more containers, a therapeutically effective amount of a fabric acid or statin derivative compounds or compositions as described in detail herein, and a label or packaging insert containing instructions for use are disclosed.

In other aspects, the present application provides methods for treating and/or preventing a variety of diseases or disorders associated with aging, stress, diabetes, obesity, neurodegenerative diseases, cardiovascular disease, blood clotting disorders, inflammation, and cancer.

These, and other objects, features and advantages of this disclosure will become apparent from the following detailed description of the various aspects of the disclosure taken in conjunction with the accompanying Examples.

### BRIEF DESCRIPTION OF THE DRAWINGS

FIG. 1. LDL Analysis in Rat High Fat, High Cholesterol Diet Model for Compounds 10A and 5A.

FIG. 2. VLDL Analysis in Rat High Fat, High Cholesterol Diet Model for Compounds 10A and 5A.

FIG. 3. HDL Analysis in Rat High Fat, High Cholesterol Diet Model for Compounds 10A and 5A.

FIG. 4. LDL Analysis in Rat High Fat, High Cholesterol Diet Model for Compound 13 and fenofibrate.

FIG. 5. VLDL Analysis in Rat High Fat, High Cholesterol Diet Model for Compound 13 and fenofibrate.

FIG. 6. HDL Analysis in Rat High Fat, High Cholesterol Diet Model for Compound 13 and fenofibrate.

FIG. 7. Blood Glucose Analysis in the Obese (ob/ob) Mouse Model.

FIG. 8. Fenofibric acid pharmacokinetic profile.

FIGS. 9A and 9B. Resveratrol pharmacokinetic profile.

### DETAILED DESCRIPTION

The present application is based in part on the use of fibric acid or statin derivatives alone or in combination (for example, with one or more lipid altering agents or PDE inhibitors) to prevent/treat the lipid related disorders described herein.

The present application discloses compositions including at least one fibric acid or statin derivative, either alone or in combination with at least one lipid altering agent, which when administered provide an effective treatment to patients suffering from, but not limited to, fatty liver disease, hepatitis, high serum levels of total cholesterol, high serum levels of LDL, low levels of HDL, and high serum levels of triglycerides.

Accordingly, the compositions and compounds disclosed herein are useful in methods for treating or preventing: a variety of lipid metabolism disorders and associated conditions such as, for example, hyperlipidemia, hypercholesterolemia, familial hypercholesterolemia, primary heterozygous familial hypercholesterolemia, primary non-familial hypercholesterolemia, xanthoma, combined hyperlipidemia, lecithin cholesterol acyltransferase deficiency, tangier disease, abetalipoproteinemia, and fatty liver disease.

The present application also provides methods for treating or preventing a variety of disorders by administering to a patient in need thereof a therapeutically effective amount of a pharmaceutical composition as disclosed and described in detail herein.

When administered to a patient, the compounds, compositions and pharmaceutical formulations described herein can lead to one or more of: reduced blood plasma or serum concentrations of low-density lipoprotein cholesterol (LDL-C); reduced blood plasma or serum concentrations of very low-density lipoprotein cholesterol (VLDL-C); reduced blood plasma or serum concentrations of intermediate-density lipoprotein cholesterol (IDL-C); reduced concentrations of cholesterol and cholesterol ester in the blood plasma or serum; reduced blood plasma or serum concentrations of apolipoprotein B; reduced blood plasma or serum concentrations of triglycerides; increased clearance of triglycerides; increased blood plasma or serum concentrations of high density lipoprotein cholesterol (HDL-C); reduced blood plasma or serum concentrations of non high-density lipoprotein cholesterol (non HDL-C); reduced levels of lipoprotein(a) (Lp(a)); increased ratio of HDL-C to LDL-C; inhibition of saponified and/or non-saponified fatty acid synthesis; reduced blood plasma or serum concentrations apolipoprotein C-II; reduced blood plasma or serum concentrations of C-reactive protein; reduced blood plasma or serum concentrations apolipoprotein C-III; increased blood plasma or serum concentrations of HDL associated proteins (including but not limited to apo A-I, apo A-II, apo A-IV, and apo E), and increased fecal excretion of cholesterol.

The compounds, compositions and pharmaceutical formulations described herein may not antagonize liver X-receptor (LXR) activity. When administered to a patient, the compounds, compositions and pharmaceutical formulations described herein may not substantially increase liver function test levels (e.g. alanine aminotransferase (ALT) and/or alanine aminotransferase (AST) levels). When administered to a patient, the compounds, compositions and pharmaceutical formulations described herein may be administered without food.

As employed above and throughout the disclosure, the following terms are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art unless otherwise indicated. As used herein and in the appended claims, the singular forms include plural referents unless the context clearly dictates otherwise. Thus, for example, reference to “a fibric acid or statin derivative” includes one or more of such compositions, as would be known to those skilled in the art.

A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations” is incorporated herein by reference.

As used herein, “Alkyl” refers to saturated hydrocarbon residues containing eight or fewer carbons in straight or branched chains, as well as cyclic structures. “Aryl” includes phenyl, substituted phenyl, naphthyl and the like; “heteroaryl” means a 5- or 6-membered aromatic heterocyclic group containing up to three heteroatoms, each selected from N, O and S. Examples include, but are not limited to thiazolyl, oxazolyl, pyridyl, furanyl, pyrrolyl, thienyl and the like.

“Acyl” refers to groups of from 1 to 8 carbon atoms of a straight, branched, cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons.

As utilized herein, the term “lower alkyl”, alone or in combination, means an acyclic alkyl radical containing from 1 to about 10, preferably from 1 to about 8 carbon atoms and more preferably from 1 to about 6 carbon atoms. Examples of such radicals include methyl, ethyl, n-propyl, isopropyl, n-butyl, isobutyl, sec-butyl, tert-butyl, pentyl, iso-amyl, hexyl, octyl and the like.

The term “lower alkenyl” refers to an unsaturated acyclic hydrocarbon radical in so much as it contains at least one double bond. Such radicals containing form about 2 to about 10 carbon atoms, preferably from about 2 to about 8 carbon atoms and more preferably 2 to about 6 carbon atoms. Examples of suitable alkenyl radicals include propylenyl, buten-1-yl, isobutenyl, penten-1-yl, 2-methylbuten-1-yl, 3-methylbuten-1-yl, hexen-1-yl, hepten-1-yl, and octen-1-yl, and the like.

The term “heterocycle” means an unsaturated cyclic compound with 1 to 6 carbon atoms and 1 to 4 heteroatoms chosen from the group consisting of nitrogen, oxygen and sulfur. The heterocycle may be fused to an aromatic hydrocarbon radical. Suitable examples include pyrrolyl, pyridinyl, pyrazolyl, triazolyl, pyrimidinyl, pyridazinyl, oxazolyl, tetrazolyl, thiazolyl, imidazolyl, indolyl, thiophenyl, furanyl, tetrazolyl, 2-pyrrolinyl, 3-pyrrolinyl, pyrrolindinyl, 1,3-dioxolanyl, imidazolinyl, imidazolidinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dithianyl, thiomorpholinyl, pyrazinyl, piperazinyl, 1,3,5-triazinyl, 1,2,5-trithianyl, benzo(b)thiophenyl, benzimidazolyl, quinolinyl, and the like.

The term “aryl” means an aromatic hydrocarbon radical of 4 to about 16 carbon atoms, preferable 6 to about 12 carbon atoms, more preferably 6 to about 10 carbon atoms. Examples of suitable aromatic hydrocarbon radicals include phenyl and naphthyl.

The terms “cycloalkyl” or “cycloalkenyl” means an alicyclic radical in a ring with 3 to 10 carbon atoms, and preferably from 3 to 6 carbon atoms. Examples of suitable alicyclic radicals include cyclopropyl, cyclopropenyl, cyclobutyl, cyclopentyl, cyclohexyl, cyclohexenyl and the like.

The term “halogen” means fluorine, chlorine, bromine or iodine.

The term “alkoxy”, alone or in combination, means an alkyl ether radical wherein the term alkyl is as defined above and in certain embodiments containing from 1 to 6 or from 1 to 4 carbon atoms. Examples of suitable alkyl ether radicals include methoxy, ethoxy, n-propoxy, isopropoxy, n-butoxy, iso-butoxy, sec-butoxy, tert-butoxy, n-pentyloxy, cyclohexyloxy, and the like.

Substituted alkyl, aryl, cycloalkyl, heterocyclyl etc. refer to alkyl, aryl, cycloalkyl, or heterocyclyl wherein up to three H atoms in each residue are replaced with halogen, haloalkyl, hydroxy, loweralkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “a residue of an amino acid” or “an amino acid residue” as used herein, refers to an amino acid (for example, as defined herein) minus the elements of water that are eliminated in forming the claimed parent molecule. For example, in the parent molecule illustrated below:

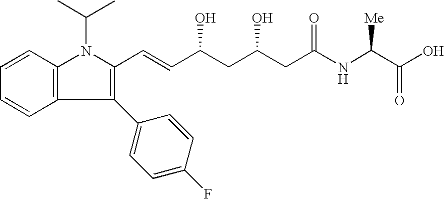

after one subtracts the atorvastatin portion, the structure that remains is:

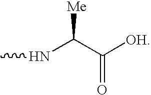

This is not sensu stricto the amino acid alanine, since it lacks the second hydrogen molecule at the point of attachment indicated by [symbol] . This and similar structures of amino acids that lack functional groups at the points of attachment are referred to herein as “residues of amino acids” or “amino acid residues.” One of skill in the art might also refer to them as “amino acid fragments.”

Similarly, the term “a residue of a statin” or “a statin residue” refers to a statin (as defined herein) minus the functional groups that are involved in the bond to the parent molecule. For example, in the parent molecule illustrated below:

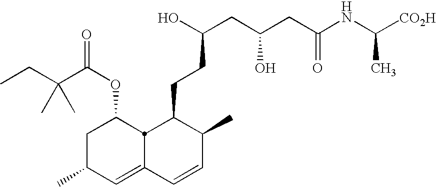

after one subtracts the alanine portion of the molecule, the structure that remains is:

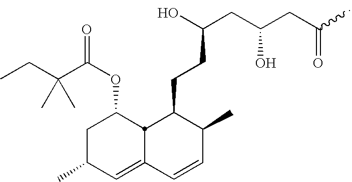

This is not sensu stricto the statin rosuvastatin, since it lacks the OH group at the point of attachment indicated by [symbol] . This and similar structures of statins that lack functional groups at the points of attachment are referred to herein as “a residue of a statin” or “statin residues.” One of skill in the art might also refer to them as “statin fragments.”

The same logic is applied to the terms “a fibric acid residue,” “a fibrate residue,” or “a residue of a fibrate”; “a residue of resveratrol” or “a resveratrol residue”; and “an imidazoline receptor agonist residue” or “a residue of an imidazoline receptor agonist” as used herein. In general, the claimed molecule is formally comprised of two or more units, termed residues that are joined with the elimination of one mole of water from their respective parents. As used herein the term “resveratrol residue” is intended to encompass resveratrol as well as resveratrol residues in which the phenolic hydroxyl residues are esterified or etherified (such as in pterostilbene). In certain embodiments the esterified or etherified resveratrol residues can have ether or ester residues of the same or different lengths. For example, in one embodiment the ester residue is —O(C₁-C₆) acyl. In another embodiment, for example, the ester residue can be any of —O(C₁) acyl; —O(C₂) acyl; —O(C₃) acyl; —O(C₄) acyl; —O(C₅) acyl; or —O(C₆) acyl. Similarly, the ether residue is —O(C₁-C₆) alkyl in an embodiment. In one embodiment, for example, the ether residue can be any of —O(C₁) alkyl; —O(C₂) alkyl; —O(C₃) alkyl; —O(C₄) alkyl; —O(C₅) alkyl; or —O(C₆) alkyl.

Amino acids include, but are not limited to, alanine, asparagine, N-β-trityl-asparagine, aspartic acid, aspartic acid-β-t-butyl ester, arginine, N^{g}-Mtr-arginine, cysteine, S-trityl-cysteine, glutamic acid, glutamic acid-γ-t-butyl ester, glutamine, N-γ-trityl-glutamine, glycine, histidine, N^{im}-trityl-histidine, isoleucine, leucine, lysine, N^{ε}-Boc-lysine, methionine, phenylalanine, proline, serine, O-t-butyl-serine, threonine, tryptophan, Nⁱⁿ-Boc-tryptophan, tyrosine, valine, sarcosine, L-alanine, chloro-L-alanine, 2-aminoisobutyric acid, 2-(methylamino)isobutyric acid, D, L-3-aminoisobutyric acid, (R)-(−)-2 aminoisobutyric acid, (S)-(+)-2-aminoisobutyric acid, D-leucine, L-leucine, D-norvaline, L-norvaline, L-2-amino-4-pentenoic acid, D-isoleucine, L-isoleucine, D-norleucine, 2,3-diaminopropionic acid, L-norleucine, D,L-2-aminocaprylic acid, β-alanine, D,L-3-aminobutyric acid, 4-aminobutyric acid, 4-(methylamino)butyric acid, 5-aminovaleric acid, 5-aminocaproic acid, 7-aminoheptanoic acid, 8-aminocaprylic acid, 11-aminodecanoic acid, 12-aminododecanoic acid, carboxymethoxylamine, D-serine, D-homoserine, L-homoserine, D-allothreonine, L-allothreonine, D-threonine, L-threonine, D,L-4-amino-3-hydroxybutyric acid, D-,L-3-hydroxynorvaline, (3S,4S)-(−)-statine, 5-hydroxy-D,L-lysine, 1-amino-1-cyclopropanecarboxylic acid, 1-amino-1-cyclopentanecarboxylic acid, 1-amino-1-cyclohexanecarboxylic acid, 5-amino-1,3-cyclohexadiene-1-carboxylic acid, 2-amino-2-norbornanecarboxylic acid, (S)-(−)-2-azetidinecarboxylic acid, cis-4-hydroxy-D-proline, cis-4-hydroxy-L-proline, trans-4-hydroxy-L-proline, 3,4-dehydro-D,L-proline, 3,4-dehydro-L-proline, D-pipecolinic acid, L-pipecolinic acid, nipecotic acid, isonipecotic acid, mimosine, 2,3-diaminopropionic acid, D,L-2,4-diaminobutyric acid, (S)-(+)-diaminobutyric acid, D-ornithine, L-ornithine, 2-methylomithine, N-ε-methyl-L-lysine, N-methyl-D-aspartic acid, D,L-2-methylglutamic acid, D,L-2-aminoadipic acid, D-2-aminoadipic acid, L-2-aminoadipic acid, (+/−)-3-aminoadipic acid, D-cysteine, D-penicillamine, L-penicillamine, D,L-homocysteine, S-methyl-L-cysteine, L-methionine, D-ethionine, L-ethionine, S-carboxymethyl-L-cysteine, (S)-(+)-2-phenylglycine, (R)-(−)-2-phenylglycine, N-phenylglycine, N-(4-hydroxyphenyl)glycine, D-phenylalanine, thienylalanine, (S)-(−)indoline-2-carboxylic acid, α-methyl,D,L-phenylalanine, β-methyl-D,L-phenylalanine, D-homophenylalanine, L-homophenylalanine, D,L-2-fluorophenylglycine, D,L-2-fluorophenylalanine, D,L-3-fluorophenylalanine, D,L-4-fluorophenylalanine, D,L-4-chlorophenylalanine, L-4-chlorophenylalanine, 4-bromo-D,L-phenylalanine, 4-iodo-D-phenylalanine, 3,3N,5-triiodo-L-thyronine, (+)-3,3N,5-triiodo-L-thyronine, D-thyronine, L-thyronine, D,L-m-tyrosine, D-4-hydroxyphenylglycine, D-tyrosine, L-tyrosine, O-methyl-L-tyrosine, 3-fluoro-D,L-tyrosine, 3-iodo-L-tyrosine, 3-nitro-L-tyrosine, 3,5-diiodo-L-tyrosine, D,L-dopa, L-dopa, 2,4,5-trihydroxyphenyl-D,L-alanine, 3-amino-L-tyrosine, 4-amino-D-phenylalanine, 4-amino-L-phenylalanine, 4-amino-D,L-phenylalanine, 4-nitro-L-phenylalanine, 4-nitro-D,L-phenylalanine, 3,5-dinitro-L-tyrosine, D,L-α-methyltyrosine, L-α-methyltyrosine, (−)-3-(3,4-dihydroxyphenyl)-2-methyl-L-alanine, D,L-threo-3-phenylserine, trans-4-(aminomethyl)cyclohexane carboxylic acid, 4-(aminomethyl)benzoic acid, D,L-3-aminobutyric acid, 3-aminocyclohexane carboxylic acid, cis-2-amino-1-cyclohexane carboxylic acid, γ-amino-β-(p-chlorophenyl) butyric acid (Baclofen), D,L-3-aminophenylpropionic acid, 3-amino-3-(4-chlorophenyl) propionic acid, 3-amino-3-(2-nitrophenyl)propionic acid, and 3-amino-4,4,4-trifluorobutyric acid.

As used herein, the term “guanidine” refers to a common functional group with the general structure (N)(N)C═N. The central bond within this group is that of an imine; the other recognizable motif within this group is an animal. “Guanidine” can include, for example, 2-aminoguanidine and methyl guanidine.

The terms “fibric acid,” “fibrate,” “fibric acid compound,” “fibrate compound,” and “fibric acid derivative” are used interchangeably throughout the specification and claims and refer to all compounds having as a core structure a 2-oxy-2-methylpropanoic acid, such as any PPARα agonist known to those skilled in the art. Suitable PPARα agonists for use with the compounds described herein include: those disclosed in U.S. Pat. No. 6,028,109 (fluorophenyl compounds), WO00/75103 (substituted phenylpropionic compounds), WO98/43081 and compounds such as beclofibrate, benzafibrate, bezafibrate (C.A.S. Registry No. 41859-67-0, see U.S. Pat. No. 3,781,328), binifibrate (C.A.S. Registry No. 69047-39-8, see BE884722), ciprofibrate (C.A.S. Registry No. 52214-84-3, see U.S. Pat. No. 3,948,973), clinofibrate (C.A.S. Registry No. 30299-08-2, see U.S. Pat. No. 3,716,583), clofibrate (such as ethyl 2-(p-chlorophenoxy)-2-methyl-propionate, e.g. Atromid-S® capsules (Wyeth-Ayerst), etofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g. Lopid® tablets (Parke Davis)), lifibrol, GW 7647, BM 170744, LY518674 and those compounds disclosed in WO03/033456, WO03/033481, WO03/043997, WO03/048116, WO03/053974, WO03/059864, and WO03/05875. Those of skill in the art would recognize that any of these fibrates can be used to formulate the fibric acid derivative compounds according to the methods disclosed herein.

The terms “statin,” “statins,” “statin compound” and “statin derivative” are used interchangeably throughout the specification and claims and refer to all compounds having as a core structure any HMG-CoA reductase inhibitor (statin compound) that is known to those skilled in the art to which this disclosure pertains. Suitable HMG-CoA reductase inhibitors for use with the compounds described herein include: atorvastatin (LIPITOR®; disclosed in U.S. Pat. No. 4,681,893, U.S. Pat. No. 5,385,929 and U.S. Pat. No. 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, crilvastatin, dalvastatin (disclosed in EP738510A2), fluvastatin (LESCOL®; disclosed in U.S. Pat. No. 4,739,073 and U.S. Pat. No. 534,772), glenvastatin, fluindostatin (disclosed in EP363934A1), velostatin (visinolin; disclosed in U.S. Pat. No. 4,448,784 and U.S. Pat. No. 4,450,171), lovastatin (mevinolin; MEVACOR® (Merck and Co.) and related compounds disclosed in U.S. Pat. No. 4,231,938), mevastatin (and related compound disclosed in U.S. Pat. No. 3,983,140), compactin (and related compounds disclosed in U.S. Pat. No. 4,804,770), pitavastatin (also known as NK-104, itavastatin, nisvastatin, nisbastatin disclosed in U.S. Pat. No. 5,102,888), pravastatin (PRAVACHOL® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin (CRESTOR®; also known as ZD-4522 disclosed in U.S. Pat. No. 5,260,440), atavastatin, visastatin, simvastatin (ZOCOR® (Merck and Co.) and related compounds as disclosed in U.S. Pat. No. 4,448,784 and U.S. Pat. No. 4,450,171), sirrivastatin, CI-981, compounds disclosed in WO03/033481, U.S. Pat. No. 4,231,938, U.S. Pat. No. 4,444,784, U.S. Pat. No. 4,647,576, U.S. Pat. No. 4,686,237, U.S. Pat. No. 4,499,289, U.S. Pat. No. 4,346,227, U.S. Pat. No. 5,753,675, U.S. Pat. No. 4,613,610, EP0221025, and EP491226, and optical or geometric isomers thereof, and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzim-idazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate. Other suitable statins, or statin prodrugs, are disclosed in PCT/US2006/018616, filed May 15, 2006, which is incorporated herein by reference. Those of skill in the art would recognize that any of these statins can be used to formulate the statin derivative compounds according to the methods disclosed herein.

Most of the compounds described herein contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as, their racemic and optically pure forms. Optically active (R)- and (S)-, or (D)- and (L)-isomers may be prepared using chiral synthons or chiral reagents, or resolved using conventional techniques. When the compounds described herein contain olefinic double bonds or other centers of geometric asymmetry, and unless specified otherwise, it is intended that the compounds include both E and Z geometric isomers. Likewise, all tautomeric forms are also intended to be included.

Terminology related to “protecting”, “deprotecting” and “protected” functionalities occurs throughout this application. Such terminology is well understood by persons of skill in the art and is used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the present disclosure, the person of ordinary skill can readily envision those groups that would be suitable as “protecting groups”.

In the case of the present disclosure, the functionalities that must be protected are carboxylic acids and alcohols. Suitable groups for that purpose are discussed in standard textbooks in the field of chemistry, such as Protective Groups in Organic Synthesis by T. W. Greene [John Wiley & Sons, New York, 1991], which is incorporated herein by reference. Particular attention is drawn to the chapters entitled “Protection for the Hydroxyl Group, Including 1,2- and 1,3-Diols” (pages 10-86) and “Protection for the Carboxyl Group” (pages 152-end).

As used herein, “treating” or “treatment of” a condition or subject refers to taking steps to obtain beneficial or desired results, including clinical results. For purposes of this disclosure, beneficial or desired clinical results include, but are not limited to, alleviation or amelioration of one or more disease, symptom, or condition related to lipid metabolism disorders, fatty liver disease, hepatitis, or erectile dysfunction.

As used herein, a “therapeutically effective amount” of a drug or pharmaceutical composition or formulation, or agent, described herein is an amount of a drug or agent that, when administered to a subject with a disease or condition, will have the intended therapeutic effect, e.g., alleviation, amelioration, palliation or elimination of one or more manifestations of the disease or condition in the subject. The full therapeutic effect does not necessarily occur by administration of one dose and may occur only after administration of a series of doses. Thus, a therapeutically effective amount may be administered in one or more administrations.

As used herein, a “prophylactically effective amount” of a drug or pharmaceutical composition or formulation, or agent, described herein is an amount of a drug or agent that, when administered to a subject, will have the intended prophylactic effect, e.g., preventing or delaying the onset (or reoccurrence) of disease or symptoms, or reducing the likelihood of the onset (or reoccurrence) of disease or symptoms. The full prophylactic effect does not necessarily occur by administration of one dose and may occur only after administration of a series of doses. Thus, a prophylactically effective amount may be administered in one or more administrations.

As used herein, and as would be understood by the person of skill in the art, the recitation of “a compound” or “a composition” is intended to include salts, solvates and inclusion complexes of that compound as well as any stereoisomeric form, or a mixture of any such forms of that compound in any ratio.

The term “pharmaceutically acceptable salt” refers to salts prepared from pharmaceutically acceptable non-toxic acids or bases including inorganic acids and bases and organic acids and bases. When the compounds of the present disclosure are basic, salts may be prepared from pharmaceutically acceptable non-toxic acids including inorganic and organic acids. Suitable pharmaceutically acceptable acid addition salts for the compounds of the present disclosure include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. When the compounds contain an acidic side chain, suitable pharmaceutically acceptable base addition salts for the compounds of the present disclosure include metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine.

Administration of any of the compositions or formulations described in detail herein includes parallel administration (i.e., administration of elements of the formulation to the subject over a period-of time), co-administration or sequential administration (in which elements of the formulation are administered at approximately the same time, e.g., within about a few seconds to a few hours of one another), and simultaneous or co-formulation (in which elements of the formulation are combined or compounded into a single dosage form suitable for oral or parenteral administration).

Combination therapy can be achieved by administering two or more agents, e.g., a fibric acid, a statin, a fibric acid derivative or a statin derivative in combination with one or more agents chosen from lipid altering agents, resveratrol, imidazoline receptor agonists, and PDE inhibitors, each of which is formulated and administered separately, or by administering two or more agents in a single formulation. For example, a fibric acid compound or a statin compound can be combined with one or more of (1) an amino-containing compound such as, but not limited to, aminoguanidine, agmatine, or amino-tetrazole, (2) resveratrol and (3) an imidazoline receptor agonist such as, but not limited to, LNP509, S-21663, S-22068, or S-23515. This combination can be accomplished by addition of the separate agents or by direct chemical coupling of the agents as disclosed herein to form a single compound. One or more lipid altering agents and/or PDE inhibitors can also be included in the fibric acid/statin/(amino-containing compound/resveratrol/imidazoline receptor agonist) combination. Each of the agents can be formulated and administered separately, or in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

A “subject” or “patient” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

A “susceptible individual” or “patient in need thereof” is an individual who suffers from, is suffering from, or is likely to or predisposed to suffer from a disorder or associated condition contemplated of being treated by the compositions described in detail herein. In humans these conditions may include, for example, dyslipidemia, hyperlipidemia, hypercholesterolemia, hypertriglyceridemia, sitosterolemia, erectile dysfunction, fatty liver disease, and hepatitis.

As used herein, the term “nitric oxide donor” is also interchangeably used herein and in the art with “NO prodrugs” or “NO-donating agents,” which refer to compounds that release free nitric oxide when administered to a patient, compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450.

The fibric acid or statin derivative compositions disclosed herein can be used for preventing and/or treating a “lipid related disorder” or “lipid metabolism disorder” including, for example: reducing blood plasma or serum concentrations of LDL cholesterol; reducing concentrations of cholesterol and cholesterol ester in the blood plasma or serum; reducing blood plasma or serum concentrations of apolipoprotein B; reducing blood plasma or serum concentrations of triglycerides; increasing blood plasma or serum concentrations of high density lipoprotein (HDL) cholesterol; increasing fecal excretion of cholesterol; inhibiting the absorption of or reducing plasma or tissue concentration of one or more sterols or stanols; preventing or treating sistoserolemia; preventing or treating vascular diseases/disorders and conditions (including, but not limited to, arteriosclerosis, atherosclerosis, cardiovascular disease, cerebrovascular disease, renovascular disease, mesenteric vascular disease, pulmonary vascular disease, ocular vascular disease and peripheral vascular disease), hyperlipidemia (including, but not limited to, hypercholesterolemia, hypertriglyceridemia, sitosterolemia), hypertension, angina, cardiac arrhythmias, congestive heart failure, and stroke; reducing the incidence of cardiovascular disease-related events; preventing or treating vascular conditions and associated thrombotic events and blood clotting disorders; preventing or treating inflammation (including, but not limited to, vascular inflammation); reducing blood plasma or serum concentrations of C-reactive protein; preventing, treating, or ameliorating symptoms of Alzheimer's Disease (AD) or other neurodegenerative diseases; regulating production or levels of at least one amyloid β (Aβ) peptide; regulating the amount of ApoE isoform 4 in the bloodstream and/or brain; slowing the aging process and reducing stress-related disorders; preventing or treating obesity; preventing or decreasing the incidence of xanthomas; preventing or minimizing muscular degeneration and related side effects associated with certain HMG-CoA reductase inhibitors (statins); preventing or treating diabetes and associated conditions; preventing or treating at least one autoimmune disorder; preventing or treating demyelination and associated disorders; preventing or treating cancer (including, but not limited to, cholesterol associated tumors); inhibiting the expression of at least one multiple (“multi”)-drug resistance gene or protein in an animal cell; enhancing the effectiveness of a chemotherapeutic agent in a subject having cancer; reversing a multi-drug resistance phenotype exhibited by an animal cell; and preventing or treating osteopenia disorders (bone loss disorders).

For example, in certain aspects, the application provides methods for the following: improving lipid parameters in diabetic and non-diabetic patients; improving glycemic control in both Type 2 diabetics and “pre-diabetic” individuals who exhibit elevated fasting plasma glucose and/or insulin resistance; reducing glycosylated hemoglobin levels (HbA_{1C}); lowering fasting plasma glucose (FPG) levels; reducing peak and 2-hour post-prandial glucose (PPG) levels; improving insulin sensitivity; reducing insulin resistance; and increasing insulin secretion. The methods are performed by administering to the patient in need thereof a therapeutically effective amount of one or more of the compounds, salts, and/or compositions disclosed herein.

Furthermore, based on rationale that it has been shown that improved glycemic control can reduce the risk of diabetes-associated morbidity and mortality (as demonstrated by the DCCT (Diabetes Control and Complications Trial. Diabetes. 1996 October; 45(10):1289-98), DCCT/EDIC (Diabetes Control and Complications Trial/Epidemiology of Diabetes Interventions and Complications Trial. Diabetes Care. 1999 January; 22(1):99-111), and UKPPDS (United Kingdom Prospective Diabetes Study. BMJ. 1995 Jan. 14; 310(6972):83-8) trials (amongst others), the compounds, salts, and compositions disclosed herein can be useful for reducing the risk of diabetes-associated complications including, but not limited to: development and progression of diabetic retinopathy; development of proliferative or severe non-proliferative retinopathy; albluminuria; microalbuminuria; nephropathy; kidney failure; cardiovascular disease (including non-fatal myocardial infarction (MI), stroke, or death from CVD); neuropathy; foot ulcers; amputations; hepatic steatosis; steatohepatitis; and cirrhosis

As used herein the term “lipid altering agent” or “dyslipidemia agent” refers to compounds including, but not limited to, bile acid sequestrants such as cholestyramine (a styrene-divinylbenzene copolymer containing quaternary ammonium cationic groups capable of binding bile acids, such as QUESTRAN® or QUESTRAN LIGHT® cholestyramine which are available from Bristol-Myers Squibb), colesevelam hydrochloride (such as WELCHOL® Tablets (polyallylamine hydrochloride) cross-linked with epichlorohydrin and alkylated with 1-bromodecane and (6-bromohexyl)-trimethylammonium bromide) which are available from Sankyo), colestipol (a copolymer of diethylenetriamine and 1-chloro-2,3-epoxypropane, such as COLESTID® tablets which are available from Pharmacia), dialkylaminoalkyl derivatives of a cross-linked dextran, LOCHOLEST®, DEAE-Sephadex (SECHOLEX®, POLICEXIDE®), water soluble derivatives such as 3,3-ioene, N-(cycloalkyl)alkylamines and poliglusam, insoluble quaternized polystyrenes, saponins and mixtures thereof and those bile acid sequestrants disclosed in WO97/11345, WO98/57652, U.S. Pat. No. 3,692,895, and U.S. Pat. No. 5,703,188. Suitable inorganic cholesterol sequestrants include bismuth salicylate plus montmorillonite clay, aluminum hydroxide and calcium carbonate antacids.

HMG-CoA reductase inhibitors are dyslipidemic agents that can be used in therapeutic combinations with compounds described herein. Suitable HMG-CoA reductase inhibitors for use in therapeutic combination with a compounds described herein include: atorvastatin (LIPITOR®; disclosed in U.S. Pat. No. 4,681,893, U.S. Pat. No. 5,385,929 and U.S. Pat. No. 5,686,104), atorvastatin calcium (disclosed in U.S. Pat. No. 5,273,995), dihydrocompactin, (disclosed in U.S. Pat. No. 4,450,171), bervastatin (disclosed in U.S. Pat. No. 5,082,859), carvastatin, crilvastatin, dalvastatin (disclosed in EP738510A2), fluvastatin (LESCOL®; disclosed in U.S. Pat. No. 4,739,073 and U.S. Pat. No. 534,772), glenvastatin, fluindostatin (disclosed in EP363934A1), velostatin (visinolin; disclosed in U.S. Pat. No. 4,448,784 and U.S. Pat. No. 4,450,171), lovastatin (mevinolin; MEVACOR® (Merck and Co.) and related compounds disclosed in U.S. Pat. No. 4,231,938), mevastatin (and related compound disclosed in U.S. Pat. No. 3,983,140), compactin (and related compounds disclosed in U.S. Pat. No. 4,804,770), pitavastatin (also known as NK-104, itavastatin, nisvastatin, nisbastatin disclosed in U.S. Pat. No. 5,102,888), pravastatin (PRAVACHOL® (Bristol Myers Squibb) and related compounds disclosed in U.S. Pat. No. 4,346,227), rivastatin (sodium 7-(4-fluorophenyl)-2,6-diisopropyl-5-methoxymethylpyridin-3-yl)-3,5-dihydroxy-6-heptanoate), rosuvastatin (CRESTOR®; also known as ZD-4522 disclosed in U.S. Pat. No. 5,260,440), atavastatin, visastatin, simvastatin (ZOCOR® (Merck and Co.) and related compounds as disclosed in U.S. Pat. No. 4,448,784 and U.S. Pat. No. 4,450,171), sirrivastatin, CI-981, compounds disclosed in WO03/033481, U.S. Pat. No. 4,231,938, U.S. Pat. No. 4,444,784, U.S. Pat. No. 4,647,576, U.S. Pat. No. 4,686,237, U.S. Pat. No. 4,499,289, U.S. Pat. No. 4,346,227, U.S. Pat. No. 5,753,675, U.S. Pat. No. 4,613,610, EP0221025, and EP491226, and optical or geometric isomers thereof, and nontoxic pharmaceutically acceptable salts, N-oxides, esters, quaternary ammonium salts, and prodrugs thereof. In HMG-CoA reductase inhibitors where an open-acid form can exist, salt and ester forms may preferably be formed from the open-acid, and all such forms are included within the meaning of the term “HMG-CoA reductase inhibitor” as used herein. Pharmaceutically acceptable salts with respect to the HMG-CoA reductase inhibitor includes non-toxic salts of the compounds which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, orithine, choline, N,N′-dibenzylethylenediamine, chloroprocaine, diethanolamine, procaine, N-benzylphenethylamine, 1-p-chlorobenzyl-2-pyrrolidine-1′-yl-methylbenzim-idazole, diethylamine, piperazine, and tris(hydroxymethyl)aminomethane. Further examples of salt forms of HMG-CoA reductase inhibitors may include, but are not limited to, acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynapthoate, iodide, isothionate, lactate, lactobionate, laurate, malate, maleate, mandelate, mesylate, methylsulfate, mucate, napsylate, nitrate, oleate, oxalate, pamaote, palmitate, panthothenate, phosphate/diphosphate, polygalacturonate, salicylate, stearate, subacetate, succinate, tannate, tartrate, teoclate, tosylate, triethiodide, and valerate.

Soluble guanylate cyclase modulators are dyslipidemic agents that can be used in therapeutic combinations with compounds described herein. Soluble guanylate cyclase (sGC) is a nitric oxide (NO) sensing haemprotein that has been described in many eukaryotes. In response to various stimuli sGC converts GTP into the 2^{nd} messenger cyclic cGMP. GC is a heterodimeric protein consisting of homologous alpha and beta subunits. Each subunit consists of an N-terminal domain which may bind haem-nitric oxide and/or oxygen, a central domain of unknown function, and a C-terminal consensus nucleotide cyclase domain. sGC can be activated via both nitric oxide (NO) dependent and independent manners. When NO binds to the haem prosthetic group in the beta subunit of sGC, catalysis is accelerated by 2-3 orders of magnitude. Agents that function as sGC modulators include but are not limited to: NO donors, eNOS transcriptional enhancers, haem-dependent sGC stimulators, haem-independent sGC activators and NOS substrates.

### NO Donors

NO donors are pharmacologically active substances that release NO in vivo or in vitro. There are different classes of NO donors, which include organic nitrates (e.g., nitroglycerin, isosorbides (e.g. isosorbide dinitrate, isosorbide mononitrate, isosorbide 5-mononitrate, isosorbide 2-mononitrate, CA Registry no. 16051-77-7), S-nitrosothiols, iron-nitrosyl complexes (e.g., sodium nitroprusside), sydnonimines, C-nitroso compounds, and secondary amine/NO complex ions.

Specific examples of some of the classes of NO donors named above include: Isosorbide (Dilatrate®-SR, Imdur®, Ismo®, Isordil®, Isordil® Titradose®, Monoket®), FK 409 (NOR-3); FR 144420 (NOR-4); 3-morpholinosydnonimine; Linsidomine chlorohydrate (“SIN-1”); S-nitroso-N-acetylpenicillamine (“SNAP”); AZD3582 (ClNOD lead compound), NCX 4016, NCX 701, NCX 1022, HCT 1026, NCX 1015, NCX 950, NCX 1000, NCX 1020, AZD 4717, NCX 1510/NCX 1512, NCX 2216, and NCX 4040 (all available from NicOx S.A.), S-nitrosoglutathione (GSNO), S-nitrosoglutathione mono-ethyl-ester (GSNO-ester), 6-(2-hydroxy-1-methyl-nitrosohydrazino)-N-methyl-1-hexanamine (NOC-9) or diethylamine NONOate, S-nitrosothiol, a nitrite, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea or a furoxan. Nitric oxide donors are also as disclosed in U.S. Pat. Nos. 5,155,137; 5,366,997; 5,405,919; 5,650,442; 5,700,830; 5,632,981; 6,290,981; 5,691,423; 5,721,365; 5,714,511; 6,511,911; and 5,814,666, Chrysselis et al. (2002) J Med Chem. 45:5406-9 (such as NO donors 14 and 17), and Nitric Oxide Donors for Pharmaceutical and Biological Research, Eds: Peng George Wang, Tingwei Bill Cai, Naoyuki Taniguchi, Wiley, 2005.

NO donors have a nitrate functionality within the molecule, and a nitroso functional group is present in all of these compounds. Glyceryl trinitrate (also known as GTN, nitroglycerin, nitroglycerine, and trinitrogylcerin) is the nitrate ester of glycerol. In sodiumnitroprusside (SNP) a molecule of nitric oxide is coordinatedto iron metal forming the square bipyramidal complex. 3-Morpholinosydnonimine (SIN-1) is a zwitterionic compound formed by combination of a morpholine and a sydnonimine. S-nitroso-N-acetylpenicillamine (SNAP) is an N-acetylated amino acid derivative with a nitrosothiol functional group. Diethylenetriamine/NO (DETA/NO) is a compound of nitric oxide covalently linked to diethylenetriamine. NCX 4016 is an m-nitroxymethyl phenyl ester of acetyl salicyclic acid.

The amount and duration of NO release by the respective NO donors determines their pharmacological properties. In vivo, some compounds act rapidly, and the amount of NO released is relatively small. In others, such as NCX 4016 (NO aspirin), the effect is slow and lasts for hours. The route of administration (oral and parenteral) and the duration of release of NO also differ. NO is connected with a specific molecular target; by binding to iron in the haem group of sGC, it produces cyclic guanosine monophosphate (cGMP), which activates a cascade of cellular processes.

The classic nitrovasodilators, organic nitrate and nitrite esters, including nitroglycerin, amyl nitrite, isosorbide dinitrate, isosorbide 5-mononitrate, and nicorandil, have been used for many years in the treatment of cardiovascular diseases. Their principal action is vasorelaxation/vasodilation, mediated by guanylyl cyclase activation and by direct inhibition of nonspecific cation channels in vascular smooth muscle cells (VSMCs). As such, these agents represent the prototypical form of NO-replacement therapy. All of the organic nitrate esters are prodrugs requiring enzymatic metabolism to generate bioactive NO. The major enzyme system involved is located within microsomal membranes, has an estimated apparent molecular mass of 160 kDa, and manifests enhanced activity in the presence of reducing equivalents, especially thiols, which potentiate the action of organic nitrate esters. Although the enzyme has not been more specifically characterized, growing evidence suggests that the cytochrome P-450 system, in conjunction with NADPH and glutathione-S-transferase activities, is required for the linked metabolic processes of denitration and reduction of organic nitrate esters to authentic NO.

eNOS Transcriptional Enhancers

Endothelial NO synthase is subject to physiological and pathophysiological regulation both at the transcriptional and at the post-transcriptional level. Compounds which enhance eNOS transcription are described in WO 02/064146, WO 02/064545, WO 02/064546 and WO 02/064565, and corresponding patent documents such as US2003/0008915, US2003/0022935, US2003/0022939 and US2003/0055093 for example. Other eNOS transcriptional enhancers include those described in US20050101599 (e.g. 2,2-difluorobenzo[1,3]dioxol-5-carboxylic acid indan-2-ylamide, and 4-fluoro-N-(indan-2-yl)-benzamide), and Sanofi-Aventis compounds AVE3085 and AVE9488 (CA Registry NO. 916514-70-0; Schafer et al., Journal of Thrombosis and Haemostasis 2005; Volume 3, Supplement 1: abstract number P1487).

### Haem-Dependent sGC Stimulators

Evgenov et al. (2006) Nature Reviews-Drug Discovery 5:755-768 review a novel class of haem-dependent sGC-stimulators which share several characteristics including crucial dependency on the presence of the reduced prosthetic haem moiety and strong synergistic enzyme activation when combined with NO. Haem-dependent sGC stimulators include but are not limited to:

YC-1 (see patent publications EP667345 and DE 19744026)

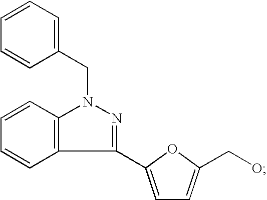

BAY 41-2272 (see patent publications DE19834047 and DE19942809)

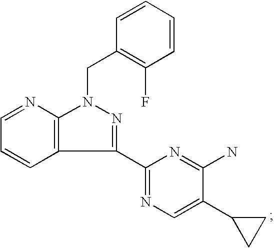

BAY 41-8543 (see patent publication DE19834044)

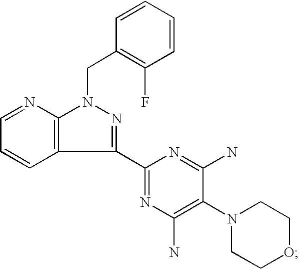

CFM-1571 (see patent publication WO2000027394)

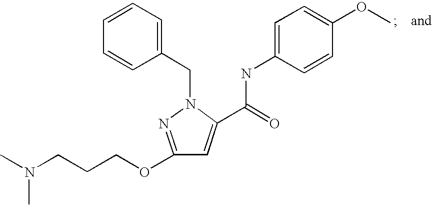

A350-619

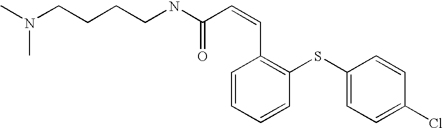

and other compounds disclosed in Tetrahedron Letters (2003), 44(48): 8661-8663.

### Haem-Independent sGC Activators

sGC can also be activated in a NO- and haem-independent manner by haem-independent sGC activators which include but are not limited to:

BAY 58-2667 (see patent publication DE19943635)

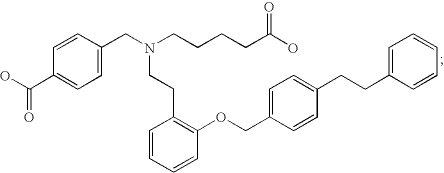

HMR-1766 (ataciguat sodium, see patent publication WO2000002851)

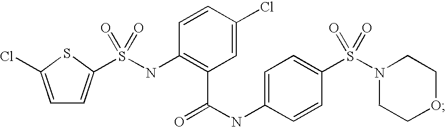

S 3448 (2-(4-chloro-phenylsulfonylamino)-4,5-dimethoxy-N-(4-(thiomorpholine-4-sulfonyl)-phenyl)-benzamide; see patent publications DE19830430 and WO2000002851)

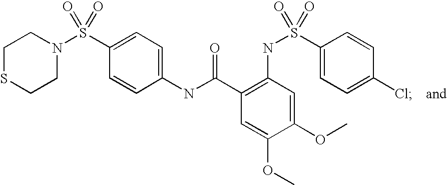

HMR-1069 (Sanofi-Aventis).

### NOS Substrates

L-arginine acts as the endogenous substrate of NOS. Other NOS substrates which can be converted to NO may also be useful in the methods described herein. NOS substrates in addition to L-arginine include n-hydroxyguanidine based analogs (such as N[G]-hydroxy-L-arginine (NOHA), (1-(3,4-dimethoxy-2-chlorobenzylideneamino)-3-hydroxyguanidine), and PR5 (1-(3,4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-caproic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear —F, —Cl, -methyl, —OH substituents, respectively); guanidine derivatives such as 3-(trifluormethyl) propylguanidine; and others reviewed in Cali et al. (2005) Current Topics in Medicinal Chemistry 5:721-736) and disclosed in the references cited therein.

Other dyslipidemic agents (e.g. lipid altering agents) which can be used in therapeutic combination with a compound described herein include:

HMG-CoA synthase inhibitors such as L-659,699 ((E,E)-11-[3′R-(hydroxy-methyl)-4′-oxo-2′R-oxetanyl]-3,5,7R-trimethyl-2,4-undecadienoic acid) and those disclosed in U.S. Pat. No. 5,120,729, U.S. Pat. No. 5,064,856, and U.S. Pat. No. 4,847,271;

cholesterol absorption inhibitors such as plant sterols, plant stanols and/or fatty acid esters of plant stanols such as sitostanol ester used in BENECOL® margarine, stanol esters, beta-sitosterol, and sterol glycosides such as tiqueside. Other cholesterol absorption inhibitors include 1,4-Diphenylazetidin-2-ones; 4-biarylyl-1-phenylazetidin-2-ones; 4-(hydroxyphenyl)azetidin-2-ones; 1,4-diphenyl-3-hydroxyalkyl-2-azetidinones; 4-biphenyl-1-phenylazetidin-2-ones; 4-biarylyl-1-phenylazetidin-2-ones; and 4-biphenylylazetidinones.

acyl coenzyme A-cholesterol acyl transferase (ACAT) inhibitors such as avasimibe (Current Opinion in Investigational Drugs. 3(9):291-297 (2003)), eflucimibe, HL-004, lecimibe, DuP-128, KY505, SMP 797, CL-277,082 (Clin Pharmacol Ther. 48(2):189-94 (1990)) and the like; and those disclosed in U.S. Pat. No. 5,510,379, WO96/26948 and WO96/10559;

CETP inhibitors such as JTT 705 identified as in Nature 406, (6792):203-7 (2000), CP 532,632, BAY63-2149, SC 591, SC 795, and the like including those described in Current Opinion in Investigational Drugs. 4(3):291-297 (2003) and those disclosed in J. Antibiot., 49(8): 815-816 (1996), and Bioorg. Med. Chem. Lett., 6:1951-1954 (1996) and patent publications U.S. Pat. No. 5,512,548, U.S. Pat. No. 6,147,090, WO99/20302, WO99/14204, WO99/41237, WO95/04755, WO96/15141, WO96/05227, WO038721, EP796846, EP818197, EP818448, DE19704244, DE19741051, DE19741399, DE197042437, DE19709125, DE19627430, DE19832159, DE19741400, JP 11049743, and JP 09059155;

squalene synthetase inhibitors such as squalestatin-1, TAK-475, and those disclosed in U.S. Pat. No. 4,871,721, U.S. Pat. No. 4,924,024, U.S. Pat. No. 5,712,396 (α-phosphono-sulfonates), Biller et al (1988) J. Med. Chem., 31:1869 (e.g. isoprenoid (phosphinyl-methyl)phosphonates), Biller et al (1996) Current Pharmaceutical Design, 2:1, P. Ortiz de Montellano et al (1977) J. Med. Chem. 20:243 (terpenoid pyrophosphates), Corey and Volante (1976) J. Am. Chem. Soc., 98:1291 (farnesyl diphosphate analog A and presqualene pyrophosphate (PSQ-PP) analogs), McClard et al (1987) J.A.C.S., 109:5544 (phosphinylphosphonates), Capson, T. L., PhD dissertation, June, 1987, Dept. Med. Chem. U of Utah, Abstract, Table of Contents, pp 16, 17, 40-43, 48-51, Summary, (cyclopropanes), Curr. Op. Ther. Patents (1993) 861, and patent publications EP0567026A1, EP0645378A1, EP0645377A1, EP0611749A1, EP0705607A2, EP0701725A1, and WO96/09827;

antioxidants such as probucol (and related compounds disclosed in U.S. Pat. No. 3,674,836), probucol derivatives such as AGI-1067 (and other derivatives disclosed in U.S. Pat. No. 6,121,319 and U.S. Pat. No. 6,147,250), tocopherol, ascorbic acid, β-carotene, selenium and vitamins such as vitamin B6 or vitamin B12 and pharmaceutically acceptable salts and esters thereof,

PPARα agonists such as those disclosed in U.S. Pat. No. 6,028,109 (fluorophenyl compounds), WO00/75103 (substituted phenylpropionic compounds), WO98/43081 and fibric acid derivatives (fibrates) such as beclofibrate, benzafibrate, bezafibrate (C.A.S. Registry No. 41859-67-0, see U.S. Pat. No. 3,781,328), binifibrate (C.A.S. Registry No. 69047-39-8, see BE884722), ciprofibrate (C.A.S. Registry No. 52214-84-3, see U.S. Pat. No. 3,948,973), clinofibrate (C.A.S. Registry No. 30299-08-2, see U.S. Pat. No. 3,716,583), clofibrate (such as ethyl 2-(p-chlorophenoxy)-2-methyl-propionate, e.g. Atromid-S® capsules (Wyeth-Ayerst), etofibrate, fenofibrate (such as Tricor® micronized fenofibrate ((2-[4-(4-chlorobenzoyl)phenoxy]-2-methyl-propanoic acid, 1-methylethyl ester; Abbott Laboratories) or Lipanthyl® micronized fenofibrate (Labortoire Founier, France)), gemcabene, gemfibrozil (such as 5-(2,5-dimethylphenoxy)-2,2-dimethylpentanoic acid, e.g. Lopid® tablets (Parke Davis)), lifibrol, GW 7647, BM 170744, LY518674 and those fibrate and fibrate acid derivatives disclosed in WO03/033456, WO03/033481, WO03/043997, WO03/048116, WO03/053974, WO03/059864, and WO03/05875;

FXR receptor modulators such as GW 4064, SR 103912, and the like;

LXR receptor modulators such as GW 3965, T9013137, and XTC0179628, and those disclosed in US20030125357, WO03/045382, WO03/053352, WO03/059874, and the like;
thyroid receptor agonists, such as QRX-401 and QRX-431 (QuatRX), GC-24 (described in US 20040110154), KB-2611 and KB-2115 (KaroBioBMS), and those disclosed in WO02/15845, WO97/21993, WO99/00353, GB98/284425, U.S. Provisional Application No. 60/183,223, and Japanese Patent Application No. JP 2000256190;
antisense inhibitors of apoB-100 or C reactive protein including, for example, ISIS 301012 and ISIS 353512 (ISIS Pharmaceuticals);

HM74 and HM74A (human HM74A is Genbank Accession No. AY148884 and rat HM74A is EMM_patAR098624) receptor agonists such as nicotinic acid (niacin) and derivatives thereof (e.g. compounds comprising a pyridine-3-carboxylate structure or a pyrazine-2-carboxylate structure, including acid forms, salts, esters, zwitterions and tautomers, where available) including but not limited to those disclosed in Wise et al (2003) J. Biol. Chem. 278: 9869 (e.g. 5-methylpyrazole-3-carboxylic acid and acifran (4,5-dihydro-5-methyl-4-oxo-5-phenyl-2-furan carboxylic acid pyradine-3-acetic acid)), as well as 5-methyl nicotinic acid, nicotinuric acid, niceritrol, nicofuranose, acipimox (5-methylpyrazine-2-carboxylic acid 4-oxide), Niaspan® (niacin extended-release tablets; Kos) and those which can be easily identified by one skilled in the art which bind to and agonize the HM74A or HM74 receptor (for example using the assays disclosed in Wise et al (2003) J. Biol. Chem 278:9869 (nicotine binding and [35S]-GTPγS binding assays), Soga et al (2003) Biochem. Biophys. Res. Comm. 303:364 (radiolabel binding assay using the HM74 receptor which could be adapted to the HM74A receptor), Tunaru et al (2003) Nature Medicine 9:352 (calcium mobilization assay using the HM74 receptor which could be adapted to the HM74A receptor) and U.S. Pat. No. 6,420,183 (FLIPR assays are described generally in and may be adapted to the HM74A or HM74 receptor);

renin angiotensin system inhibitors;

bile acid reabsorption inhibitors (bile acid reuptake inhibitors), such as BARI 1453, SC435, PHA384640, S8921, AZD7706, and the like;

PPARδ agonists (including partial agonists) such as GW 501516, and GW 590735, and those disclosed in U.S. Pat. No. 5,859,051 (acetophenols), WO03/024395, WO97/28149, WO01/79197, WO02/14291, WO02/46154, WO02/46176, WO02/076957, WO03/016291, WO03/033493, WO99/20275 (quinoline phenyl compounds), WO99/38845 (aryl compounds), WO00/63161 (1,4-disubstituted phenyl compounds), WO01/00579 (aryl compounds), WO01/12612 & WO01/12187 (benzoic acid compounds), and WO97/31907 (substituted 4-hydroxy-phenylalconic acid compound);

sterol biosynthesis inhibitors such as DMP-565;

triglyceride synthesis inhibitors;

microsomal triglyceride transport (MTTP) inhibitors, such as inplitapide, LAB687, and CP346086, AEGR 733, implitapide and the like;

HMG-CoA reductase gene expression inhibitors (e.g. compounds that decrease HMG-CoA reductase expression by affecting (e.g. blocking) transcription or translation of HMG-CoA reductase into protein or compounds that may be biotransformed into compounds that have the aforementioned attributes by one or more enzymes in the cholesterol biosynthetic cascade or may lead to the accumulation of an isoprene metabolite that has the aforementioned activities (such regulation is readily determined by those skilled in the art according to standard assays (Methods of Enzymology, 110:9-19 1985))) such as those disclosed in U.S. Pat. No. 5,041,432 (certain 15-substituted lanosterol derivatives) and E. I. Mercer (1993) Prog. Lip. Res. 32:357 (oxygenated sterols that suppress the biosynthesis of HMG-CoA reductase);

squalene epoxidase inhibitors such as NB-598 ((E)-N-ethyl-N-(6,6-dimethyl-2-hepten-4-y-nyl)-3-[(3,3′-bithiophen-5-yl)methoxy]benzene-methanamine hydrochloride);

low density lipoprotein (LDL) receptor inducers such as HOE-402 (an imidazolidinyl-pyrimidine derivative that directly stimulates LDL receptor activity, see Huettinger et al (1993) Arterioscler. Thromb. 13:1005);

platelet aggregation inhibitors;

5-LO or FLAP inhibitors;

PPAR modulators (including compounds that may have multiple functionality for activating various combinations of PPARα, PPARγ, and PPARδ) such as those disclosed in U.S. Pat. No. 6,008,237, U.S. Pat. No. 6,248,781, U.S. Pat. No. 6,166,049, WO00/12491, WO00/218355, WO00/23415, WO00/23416, WO00/23425, WO00/23442, WO00/23445, WO00/23451, WO00/236331, WO00/236332, WO00/238553, WO00/50392, WO00/53563, WO00/63153, WO00/63190, WO00/63196, WO00/63209, WO00/78312, WO00/78313, WO01/04351, WO01/14349, WO01/14350, WO01/16120, WO01/17994, WO01/21181, WO01/21578, WO01/25181, WO01/25225, WO01/25226, WO01/40192, WO01/79150, WO02/081428, WO02/100403, WO02/102780, WO02/79162, WO03/016265, WO03/033453, WO03/042194, WO03/043997, WO03/066581, WO97/25042, WO99/07357, WO99/11255, WO99/12534, WO99/15520, WO99/46232, and WO98/05331 (including GW2331 or (2-(4-[difluorophenyl]-1 heptylureido)ethyl]phenoxy)-2-methylbutyric));

niacin-bound chromium, as disclosed in WO03/039535;

substituted acid derivatives disclosed in WO03/040114;

apolipoprotein B inhibitors such as those disclosed in WO02/090347, WO02/28835, WO03/045921, WO03/047575;

Factor Xa modulators such as those disclosed in WO03/047517, WO03/047520, WO03/048081;

ileal bile acid transport (“IBAT”) inhibitors (or apical sodium co-dependent bile acid transport (“ASBT”) inhibitors) such as benzothiepines (including 1,2-benzothiazepines; 1,4-benzothiazepines; 1,5-benzothiazepines; 1,2,5-benzothiadiazepines);

PPARδ activators such as disclosed in WO01/00603 (thiazole and oxazole derivates (e.g. C.A.S. Registry No. 317318-32-4), WO97/28149 (fluoro, chloro and thio phenoxy phenylacetic), U.S. Pat. No. 5,093,365 (non-1-oxidizable fatty acid analogues), and WO99/04815. Tests showing the efficacy of the therapy and the rationale for the combination therapy with a dyslipidemic agent are presented in US20030069221 (where the dyslipidemic agents are called ‘cardiovascular agents’).

The compounds described herein can be used in therapeutic combination with one or more anti-diabetic agents, including but not limited to:

PPARγ agonists such as glitazones (e.g., balaglitazone, ciglitazone, darglitazone (CP-86325, Pfizer), englitazone (CP-68722, Pfizer), isaglitazone (MIT/J&J), MCC-555 (Mitsibishi disclosed in U.S. Pat. No. 5,594,016), pioglitazone (such as such as Actos™ pioglitazone; Takeda), rosiglitazone (Avandia™; Smith Kline Beecham), rosiglitazone maleate, troglitazone (Rezulin®, disclosed in U.S. Pat. No. 4,572,912), GL-262570 (Glaxo Welcome), BRL49653 (disclosed in WO98/05331), CLX-0921, 5-BTZD, GW-0207, LG-100641, JJT-501 (JPNT/P&U), L-895645 (Merck), R-119702 (Sankyo/Pfizer), N,N-2344 (Dr. Reddy/NN), YM-440 (Yamanouchi), LY-300512, LY-519818, R483 (Roche), T131 (Tularik), and the like and compounds disclosed in U.S. Pat. No. 5,994,554, WO97/10813, WO97/27857, WO97/28115, WO97/28137, WO97/27847, WO00/76488, WO03/000685, WO03/027112, WO03/035602, WO03/048130, WO03/055867, and pharmaceutically acceptable salts thereof,

biguanides such as metformin hydrochloride (N,N-dimethylimidodicarbonimidic diamide hydrochloride, such as Glucophage™, Bristol-Myers Squibb); metformin hydrochloride with glyburide, such as Glucovance™, Bristol-Myers Squibb); buformin (Imidodicarbonimidic diamide, N-butyl-); etoformine (1-Butyl-2-ethylbiguanide, Schering A. G.) and phenformin;

protein tyrosine phosphatase-1B (PTP-1B) inhibitors, such as A-401,674, KR 61639, OC-060062, OC-83839, OC-297962, MC52445, MC52453, ISIS 113715, and those disclosed in WO03/032916, WO03/032982, WO03/041729, WO03/055883, WO02/26707, WO02/26743, JP2002114768, and pharmaceutically acceptable salts and esters thereof,

sulfonylureas such as acetohexamide (e.g. Dymelor, Eli Lilly), carbutamide, chlorpropamide (e.g. Diabinese®, Pfizer), gliamilide (Pfizer), glibenclamide, gliclazide (e.g. Diamcron, Servier Canada Inc), glimepiride (e.g. disclosed in U.S. Pat. No. 437,978, such as Amaryl™, Aventis), glipentide, glipizide (e.g. Glucotrol or Glucotrol XL Extended Release, Pfizer), gliquidone, glisolamide, glyburide, glibenclamide (e.g. Micronase or Glynase Prestab, Pharmacia & Upjohn and Diabeta, Aventis), tolazamide (e.g. Tolinase), and tolbutamide (e.g. Orinase), and pharmaceutically acceptable salts and esters thereof,

meglitinides such as repaglinide (e.g. Pranidin®, Novo Nordisk), KAD1229 (PF/Kissei), and nateglinide (e.g. Starlix®, Novartis), and pharmaceutically acceptable salts and esters thereof,

alpha glucoside hydrolase inhibitors (or glucoside inhibitors) such as acarbose (e.g. Precose™, Bayer disclosed in U.S. Pat. No. 4,904,769), miglitol (such as GLYSET™, Pharmacia & Upjohn disclosed in U.S. Pat. No. 4,639,436), camiglibose (Methyl 6-deoxy-6-[(2R,3R,4R,5S)-3,4,5-trihydroxy-2-(hydroxymethyl)piperidino]-α-D-glucopyrano side, Marion Merrell Dow), voglibose (Takeda), adiposine, emiglitate, pradimicin-Q, salbostatin, CKD-711, MDL-25,637, MDL-73,945, and MOR 14, and the compounds disclosed in U.S. Pat. No. 4,062,950, U.S. Pat. No. 4,174,439, U.S. Pat. No. 4,254,256, U.S. Pat. No. 4,701,559, U.S. Pat. No. 4,639,436, U.S. Pat. No. 5,192,772, U.S. Pat. No. 4,634,765, U.S. Pat. No. 5,157,116, U.S. Pat. No. 5,504,078, U.S. Pat. No. 5,091,418, U.S. Pat. No. 5,217,877, U.S. Pat. No. 51,091 and WO01/47528 (polyamines);

α-amylase inhibitors such as tendamistat, trestatin, and A1-3688, and the compounds disclosed in U.S. Pat. No. 4,451,455, U.S. Pat. No. 4,623,714, and U.S. Pat. No. 4,273,765;

insulin secreatagogues such as linogliride and A-4166 and pharmaceutically acceptable salts and esters thereof,

fatty acid oxidation inhibitors, such as clomoxir, and etomoxir, and pharmaceutically acceptable salts and esters thereof;

A2 antagonists, such as midaglizole, isaglidole, deriglidole, idazoxan, earoxan, and fluparoxan, and pharmaceutically acceptable salts and esters thereof,

insulin and related compounds (e.g. insulin mimetics) such as biota, LP-100, novarapid, insulin detemir, insulin lispro, insulin glargine, insulin zinc suspension (lente and ultralente), Lys-Pro insulin, GLP-1 (1-36) amide, GLP-1 (73-7) (insulintropin, disclosed in U.S. Pat. No. 5,614,492), LY-315902 (Lilly), GLP-1 (7-36)-NH2), AL-401 (AutoImmune), certain compositions as disclosed in U.S. Pat. No. 4,579,730, U.S. Pat. No. 4,849,405, U.S. Pat. No. 4,963,526, U.S. Pat. No. 5,642,868, U.S. Pat. No. 5,763,396, U.S. Pat. No. 5,824,638, U.S. Pat. No. 5,843,866, U.S. Pat. No. 6,153,632, U.S. Pat. No. 6,191,105, and WO 85/05029, and primate, rodent, or rabbit insulin including biologically active variants thereof including allelic variants, more preferably human insulin available in recombinant form (sources of human insulin include pharmaceutically acceptable and sterile formulations such as those available from Eli Lilly (Indianapolis, Ind. 46285) as Humulin™ (human insulin rDNA origin), also see the THE PHYSICIAN'S DESK REFERENCE, 55.sup.th Ed. (2001) Medical Economics, Thomson Healthcare (disclosing other suitable human insulins);

non-thiazolidinediones such as JT-501 and farglitazar (GW-2570/GI-262579), and pharmaceutically acceptable salts and esters thereof,

PPARα/γ dual agonists such as AR-HO39242 (Aztrazeneca), GW-409544 (Glaxo-Wellcome), BVT-142, CLX-0940, GW-1536, GW-1929, GW-2433, KRP-297 (Kyorin Merck; 5-[(2,4-Dioxo thiazolidinyl)methyl]methoxy-N-[[4-(trifluoromethyl)phenyl]methyl]benzamide), L-796449, LR-90, MK-0767, SB 219994, muraglitazar, reglitazar (JTT-501) and those disclosed in WO99/16758, WO99/19313, WO99/20614, WO99/38850, WO00/23415, WO00/23417, WO00/23445, WO00/50414, WO01/00579, WO01/79150, WO02/062799, WO03/004458, WO03/016265, WO03/018010, WO03/033481, WO03/033450, WO03/033453, WO03/043985, WO 031053976 and pharmaceutically acceptable salts and esters thereof;

other insulin sensitizing drugs;

VPAC2 receptor agonists;

GLK modulators, such as those disclosed in WO03/015774;

retinoid modulators such as those disclosed in WO03/000249;

GSK 3β/GSK 3 inhibitors such as 4-[2-(2-bromophenyl)-4-(4-fluorophenyl-1H-imidazol-5-yl]pyridine and those compounds disclosed in WO03/024447, WO03/037869, WO03/037877, WO03/037891, WO03/068773, EP1295884, EP1295885, and the like;

glycogen phosphorylase (HGLPa) inhibitors such as CP-368,296, CP-316,819, BAYR3401, and compounds disclosed in WO01/94300, WO02/20530, WO03/037864, and pharmaceutically acceptable salts or esters thereof;

ATP consumption promotors such as those disclosed in WO03/007990;

TRB3 inhibitors;

vanilloid receptor ligands such as those disclosed in WO03/049702;

hypoglycemic agents such as those disclosed in WO03/015781 and WO03/040114;

glycogen synthase kinase 3 inhibitors such as those disclosed in WO03/035663;

agents such as those disclosed in WO99/51225, US20030134890, WO01/24786, and WO03/059870;

insulin-responsive DNA binding protein-1 (IRDBP-1) as disclosed in WO03/057827, and the like;

adenosine A2 antagonists such as those disclosed in WO03/035639, WO03/035640, and the like;

PPARδ agonists such as GW 501516, GW 590735, and compounds disclosed in JP10237049 and WO02/14291;

dipeptidyl peptidase IV (DP-IV) inhibitors, such as isoleucine thiazolidide, NVP-DPP728, P32/98, LAF 237, P3298, TSL225, valine pyrrolidide, TMC-2A/2B/2C, CD-26 inhibitors, FE999011, P9310/K364, VIP 0177, DPP4, SDZ 274-444, and the compounds disclosed in WO03/004498, WO03/004496, EP1258476, WO02/083128, WO02/062764, WO03/000250, WO03/002530, WO03/002531, WO03/002553, WO03/002593, WO03/000180, and WO03/000181;

GLP-1 agonists such as exendin-3 and exendin-4 (including the 39 aa peptide synthetic exendin-4 called Exenatide®), and compounds disclosed in US2003087821 and NZ 504256, and pharmaceutically acceptable salts and esters thereof,

peptides including amlintide and Symlin® (pramlintide acetate);

glycokinase activators such as those disclosed in US2002103199 (fused heteroaromatic compounds) and WO02/48106 (isoindolin-1-one-substituted propionamide compounds); and

other anti-diabetic agents such as cholestagel (Sankyo/Geltex), lipostabil (Rhone-Poulenc), Eisai E-5050 (an N-substituted ethanolamine derivative), imanixil (HOE-402), tetrahydrolipstatin (THL), istigmastanylphosphorylcholine (SPC, Roche), aminocyclodextrin (Tanabe Seiyoku), Ajinomoto AJ-814 (azulene derivative), melinamide (Sumitomo), Sandoz 58-035, American Cyanamid CL-277,082 and CL-283,546 (disubstituted urea derivatives), acipimox, acifran, neomycin, p-aminosalicylic acid, aspirin, poly(diallylmethylamine) derivatives such as disclosed in U.S. Pat. No. 4,759,923, quaternary amine poly(diallyldimethylammonium chloride), pancreatic cholesteryl hydrolase (pCEH) inhibitors (such as WAY-121898), omega 3 fatty acids, fish oil (which contains Omega 3 fatty acids (3-PUFA)), and ionenes such as disclosed in U.S. Pat. No. 4,027,009. Tests showing the efficacy of the therapy and the rationale for the combination therapy with an anti-diabetic agent are presented in US20040214811.

### Fibric Acid and Statin Derivative Compounds

In part, the present application relates to compounds represented by the structure of Formula (I) or (II):

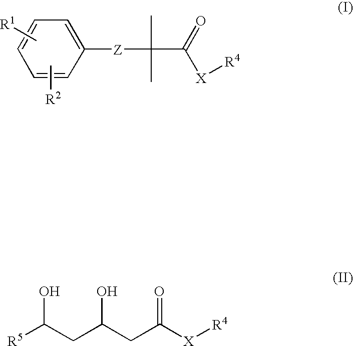

wherein

R¹ is chosen from H and halogen;

R² is chosen from H, halogen, cycloalkyl substituted with from 1 to 3 halogens, COR³, and (CH₂)ₘNHOR³;

R³ is phenyl substituted with from one to three halogen groups;

Z is chosen from O and (CH₂)ₙO;

X is chosen from direct bond, O, NH, and an amino acid residue;

R⁴ is chosen from OH, NO, NO₂, an amino acid residue, a fibric acid residue, guanidine, tetrazolyl, agmatine, an amino-containing compound, lower alkyl terminating in ONO, (ONO₂)ₚ, or guanidine, a resveratrol residue, and an imidazoline receptor agonist residue;

wherein m, n, and p are independently chosen from 1 to 3; and

R⁵ is chosen from a residue of a statin.

In certain embodiments, the application provides compounds represented by the structure of Formula (III):

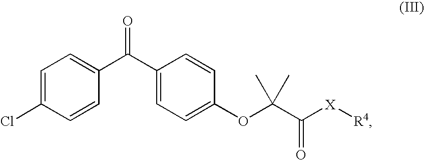

wherein

X is chosen from direct bond, O, NH, and any amino acid residue;

R⁴ is chosen from OH, NO, NO₂, any amino acid residue, guanidine, tetrazolyl, agmatine, an amino-containing compound, lower alkyl terminating in ONO, (ONO₂)ₚ, or guanidine, a resveratrol residue, and an imidazoline receptor agonist residue; and

wherein p is independently chosen from 1 to 3.

Methods of synthesis and specific experimental for representative compounds in the genus of Formula (III) include the following:

### EXAMPLE 1

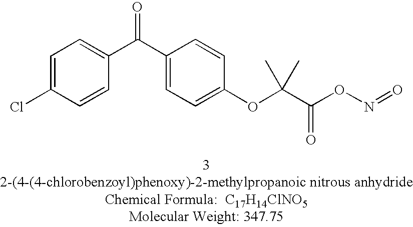

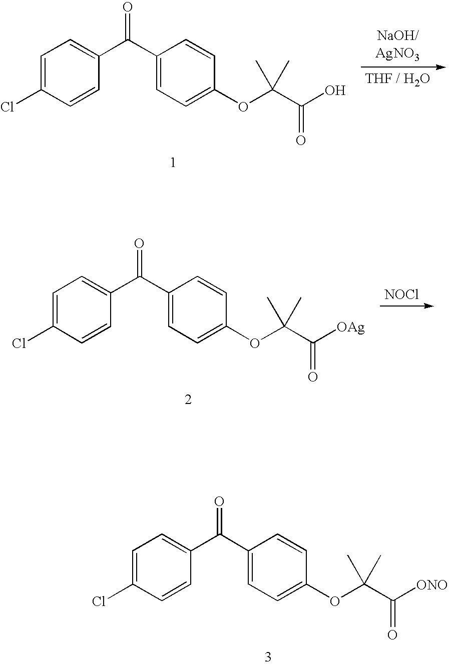

Step 1. Stromnova, Tatiana A.; Paschenko, Denis V.; Boganova, Lyubov' I.; Daineko, Mikhail V.; Katser, Sergei B.; Churakov, Andrei V.; Kuz'mina, Lyudmila G.; Howard, Judith A. K. Inorganica Chimica Acta 2003, 350 283-288.
Step 2. Using the method of Pritzkow, W.; Nitzer, H. Journal fuer Praktische Chemie (Leipzig) (1964), 25(1-2), 69-78.

### EXAMPLE 2

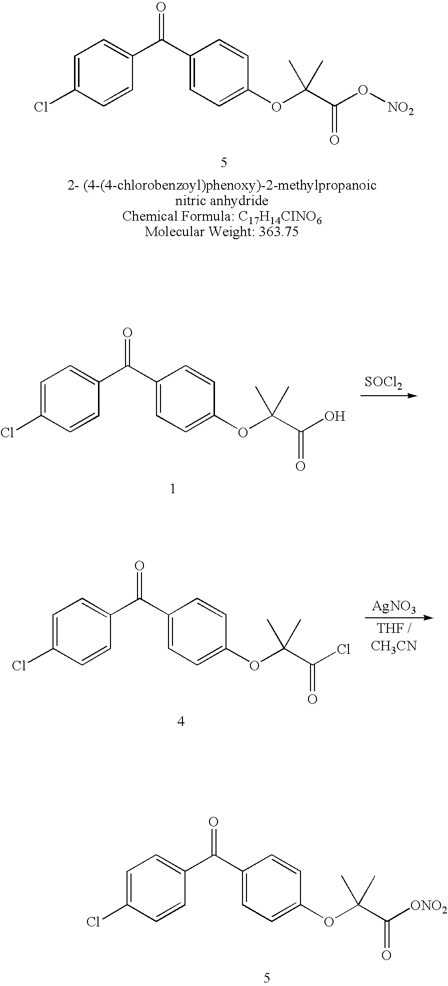

Step 1. see Lafon, L. U.S. Pat. No. 4,146,728, 1979.

### Step 2. See Burton, H.; Praill, P. F. G. Journal of the Chemical Society 1955, 729-731.

### EXAMPLE 3

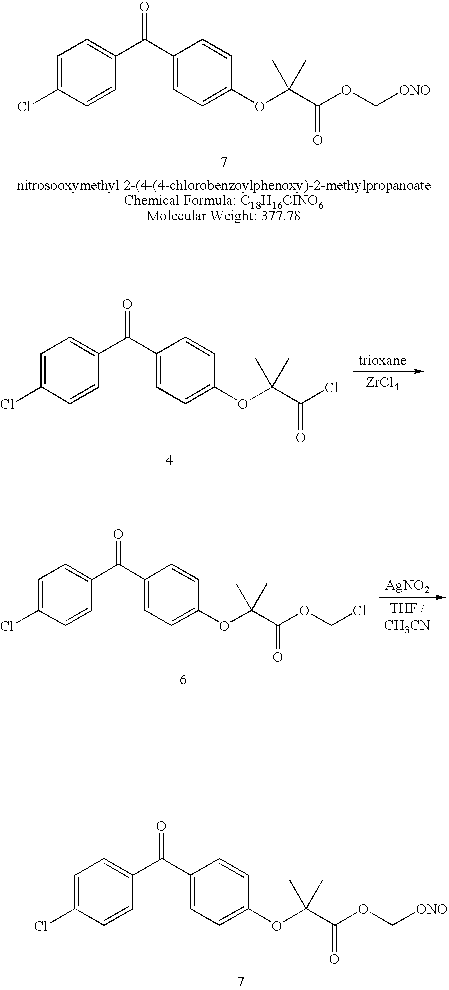

Step 1. See Mudryk, Boguslaw; Rajaraman, Shanthi; Soundararajan, Nachimuthu. Tetrahedron Letters 2002, 43(36), 6317-6318.
Step 2. Soloveichik, S. U.S. Pat. No. 2,714,606 1955.

### EXAMPLE 4

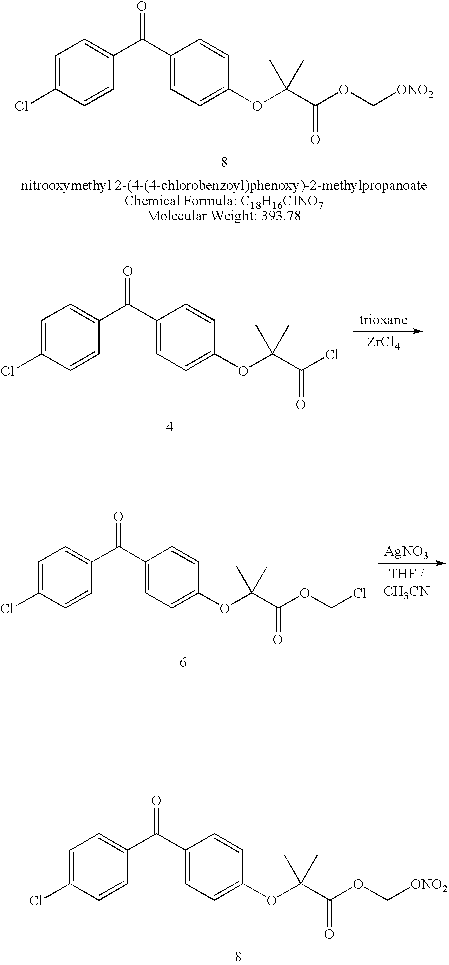

Step 1. See Mudryk, Boguslaw; Rajaraman, Shanthi; Soundararajan, Nachimuthu. Tetrahedron Letters 2002, 43(36), 6317-6318.
Step 2. Kawashima, Y.; Ikemoto, T.; Horiguchi, A.; Hayashi, M.; Matsumoto, K.; Kawarasaki, K.; Yamazaki, R.; Okuyama, S.; Hatayama, K. J. Med. Chem. 1993, 36, 815-819.

### EXAMPLE 5

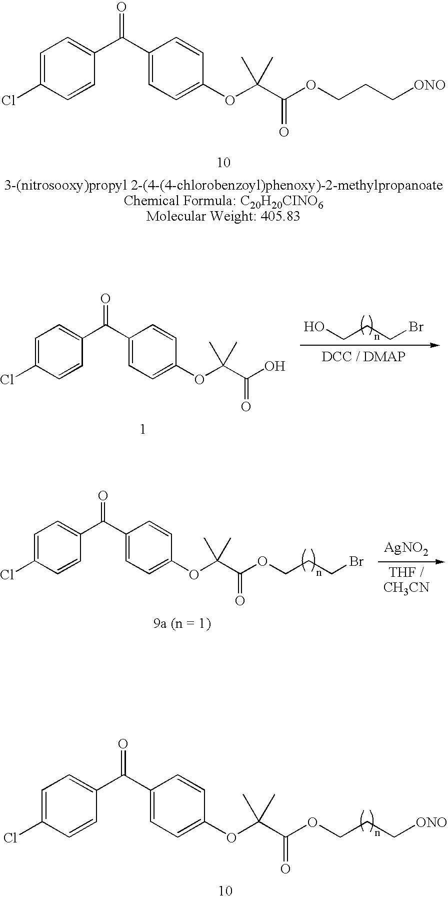

Step 1. Esterification of fenofibric acid with 3-bromopropanol.
Step 2. Soloveichik, S. U.S. Pat. No. 2,714,606 1955.

### EXAMPLE 6

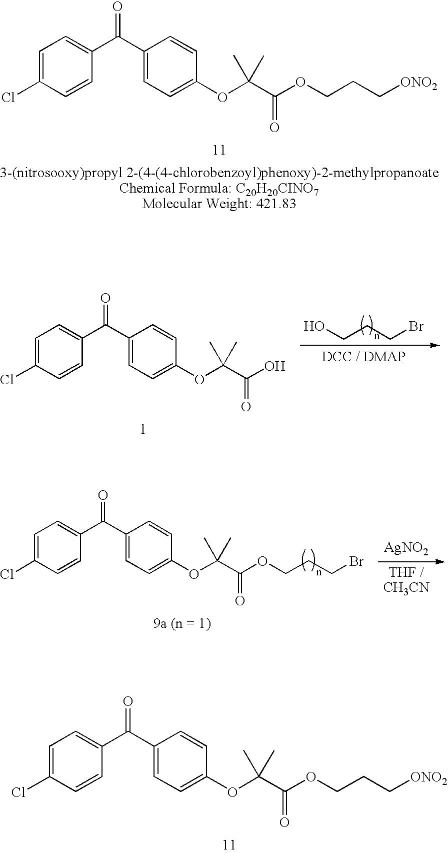

Step 1. Fenofibric acid is esterified with 3-bromopropanol (n=1) in the presence of dicyclohexylcarbodiimide (DCC) and 4-dimethylamino pyridine (DMAP).
Step 2. Reaction of the aliphatic bromide in acetonitrile gives the nitrate ester.

### EXAMPLE 7

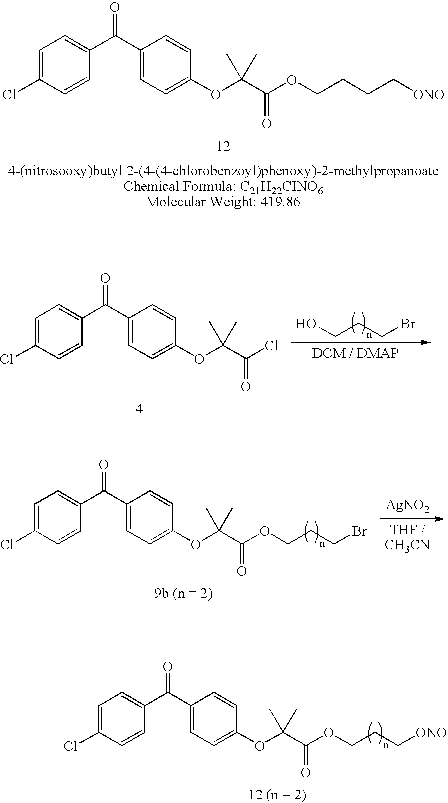

Step 1. Esterification of fenofibric acid with 4-bromobutanol (n=2).

Coupling of 4-bromobutanol (n=2) with fenofibric acid chloride (4) in dichloromethane with dimethylaminopyridine gave 9b as a tan solid that was used as is. ¹H-NMR (CDCl₃): δ 7.76-7.70 (m, 4H), 7.45 (d, 2H, J=8 Hz), 6.86 (d, 2H, J=9 Hz), 4.22-4.18 (m, 2H), 3.34-3.30 (m, 2H), 1.79-1.75 (m, 4H), 1.69 (s, 6H) ppm.

Step 2. Soloveichik, S. U.S. Pat. No. 2,714,606 1955.

### EXAMPLE 8

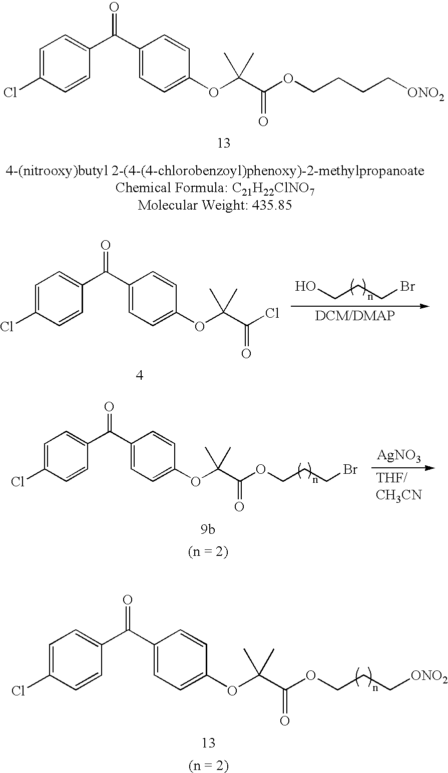

Step 1. Esterification of fenofibric acid chloride with 4-bromobutanol gave 9b (n=2).

Coupling of 4-bromobutanol (n=2) with fenofibric acid chloride (4) in dichloromethane with dimethylaminopyridine gave 9b as a tan solid that was used as is. ¹H-NMR (CDCl₃): δ 7.76-7.70 (m, 4H), 7.45 (d, 2H, J=8 Hz), 6.86 (d, 2H, J=9 Hz), 4.22-4.18 (m, 2H), 3.34-3.30 (m, 2H), 1.79-1.75 (m, 4H), 1.69 (s, 6H) ppm.

Step 2. Reaction of the aliphatic bromide in acetonitrile gave the nitrate ester.

Addition of silver nitrate to 9b in acetonitrile and heating to 50° C. for 2 days followed by chromatography (ethyl acetate/hexane) gave 13 in a yield of 86% as a clear oil which solidified to a waxy white solid. ¹H-NMR (CDCl₃): δ 7.82-7.62 (m, 4H), 7.46 (d, 2H, J=8 Hz), 6.85 (d, 2H, J=8 Hz), 4.42-4.28 (m, 2H), 4.28-4.11 (m, 2H), 1.69 (s, 6H), 1.69-1.51 (m, 4H) ppm

### EXAMPLES 9-16

The following nitrite and nitrate esters can be prepared by the protocols listed immediately above by esterification with the appropriate bromo alcohol followed by treatment with silver nitrite or silver nitrate.

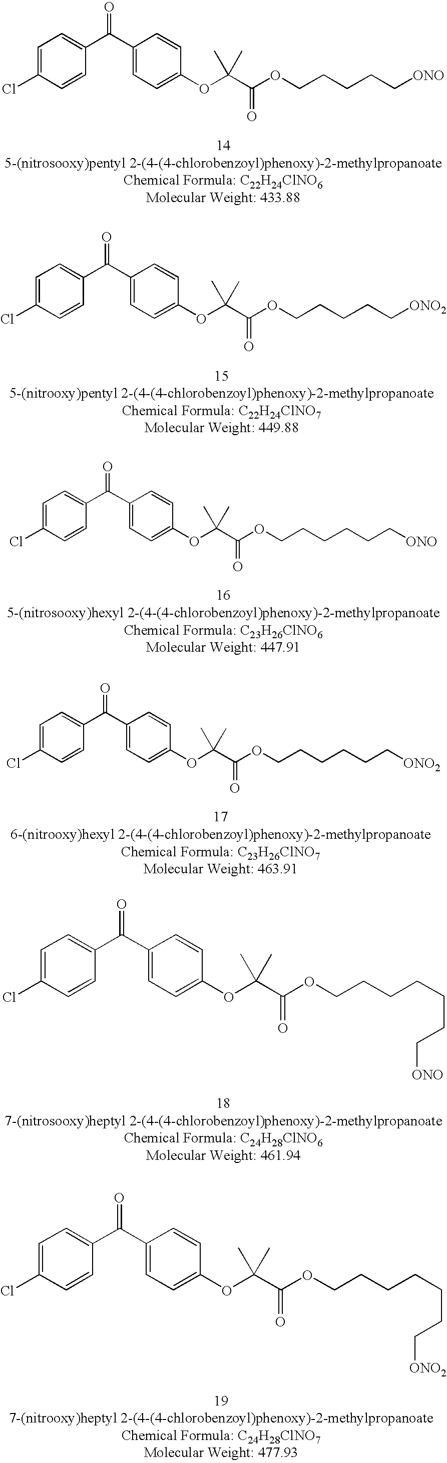

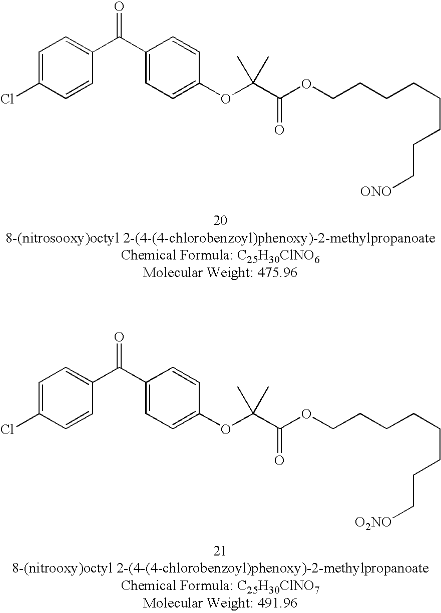

### EXAMPLE 17

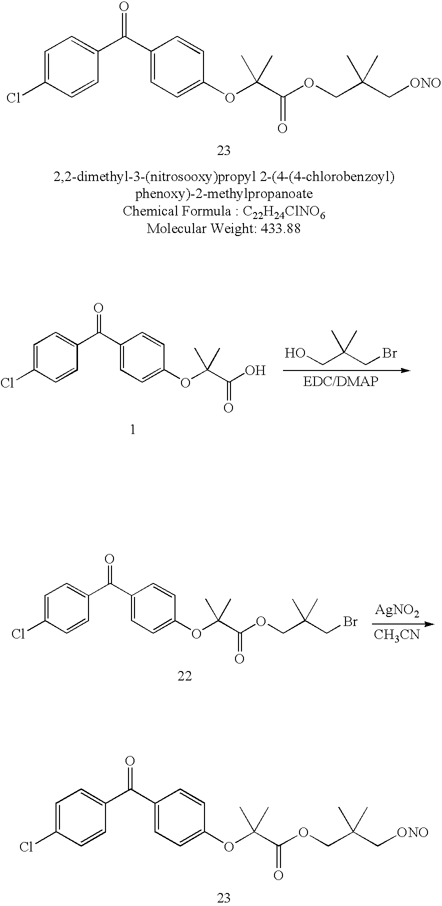

Step 1. Esterification of fenofibric acid with 3-bromo-2,2-dimethylpropan-1-ol.
Step 2. Soloveichik, S. U.S. Pat. No. 2,714,606 1955.

### EXAMPLE 18

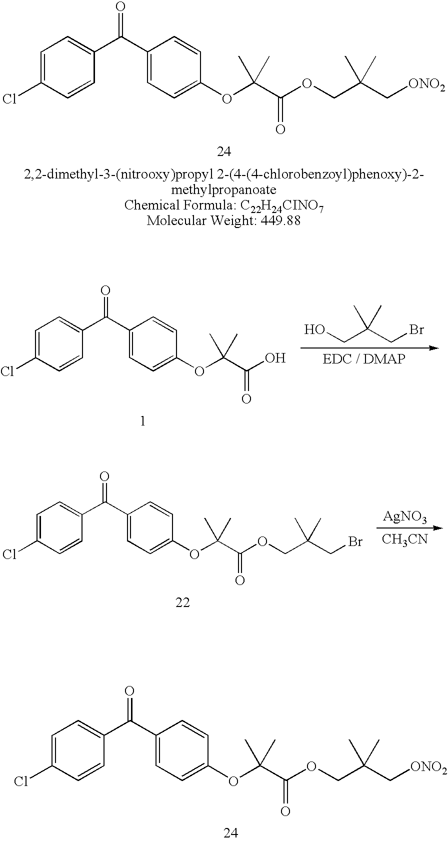

Step 1. Esterification of fenofibric acid with 3-bromo-2,2-dimethylpropan-1-ol.
Step 2. Reaction of the aliphatic bromide (22) in acetonitrile gives the nitrate ester.

### EXAMPLE 19

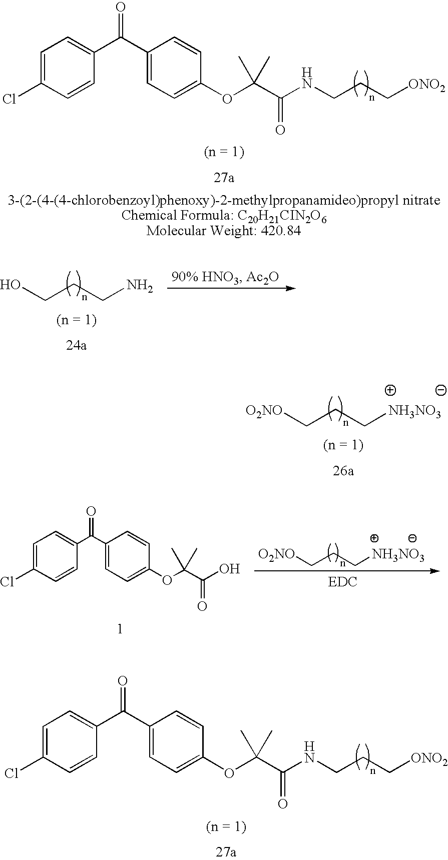

Step 1. Meyrs, G. S.; Winthrop, S. O. U.S. Pat. No. 2,975,208 1961 (n=1)
Step 2. EDC is N-(3-dimethylaminopropyl)-N′-ethylcarbodiimide hydrochloride.

### EXAMPLE 20

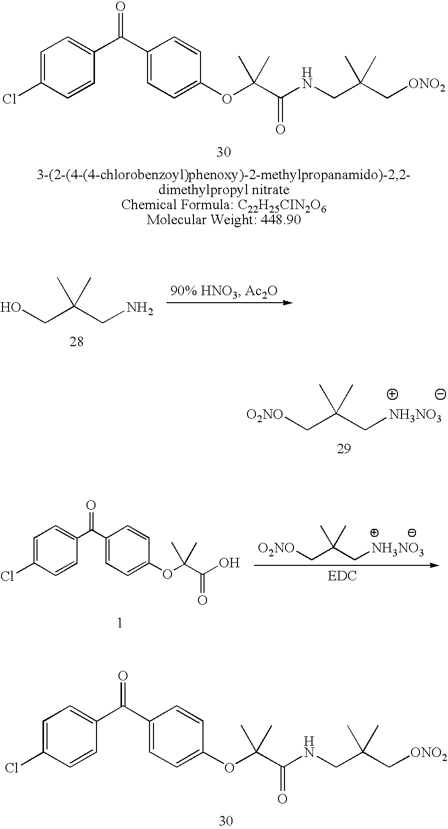

Step 1. Meyrs, G. S.; Winthrop, S. O. U.S. Pat. No. 2,975,208 1961 (n=1)
Step 2. EDC is N-(3-dimethylaminopropyl)-N′-ethylcarbodiimide hydrochloride.

### EXAMPLE 21

The following amides containing a nitrate ester can be prepared by the protocols listed immediately above by nitration of an amino alcohol with acetyl nitrate generated in situ from 90% nitric acid and acetic anhydride. Coupling of the amine nitrate ester with fenofibric acid is promoted by the coupling agent EDC to afford the desired amide derivatives, 27 b-f.

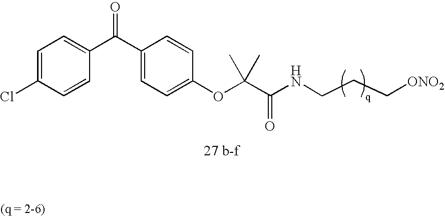

### EXAMPLE 22

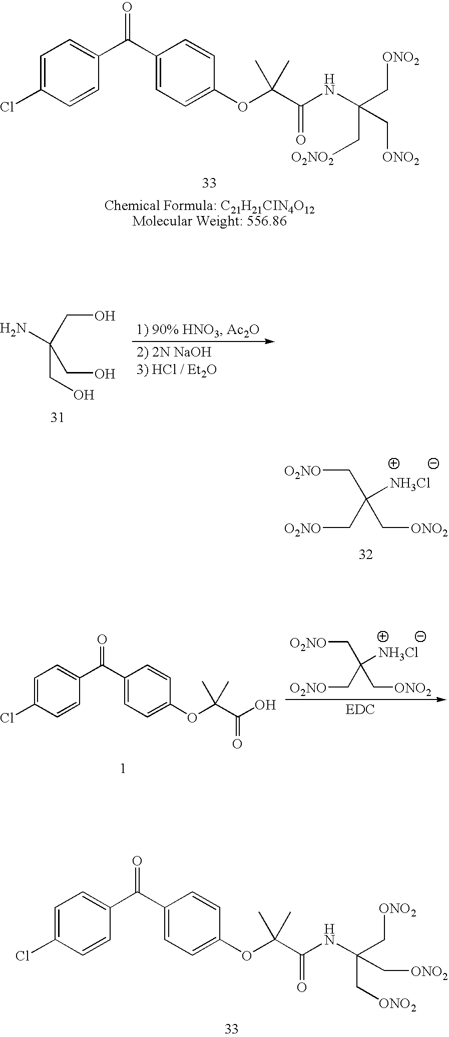

Step 1. Meyrs, G. S.; Winthrop, S. O. U.S. Pat. No. 2,975,208 1961 (n=1)
Step 2. EDC is N-(3-dimethylaminopropyl)-N′-ethylcarbodiimide hydrochloride.

### EXAMPLE 23

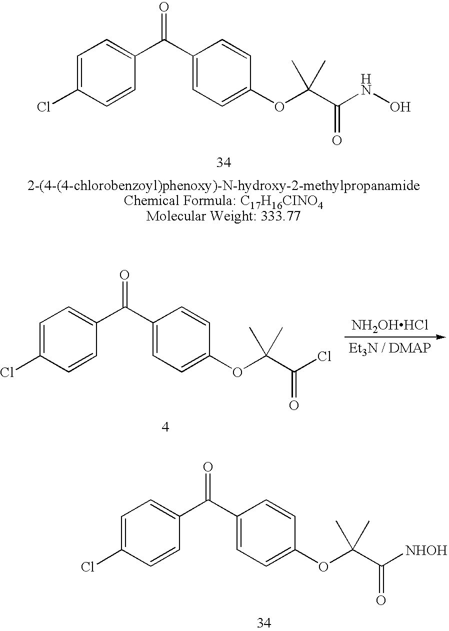

### EXAMPLE 24

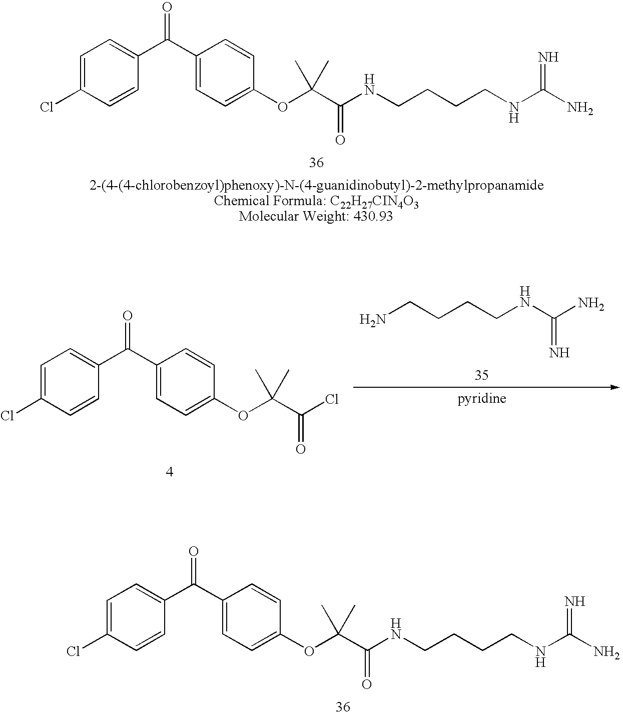

Step 1. Coupling of agmatine (35) with fenofibric acid chloride (4) in pyridine gave 36 as a white solid in 10% yield. ¹H-NMR (CDCl₃): δ 7.67 (d, 2H, J=8 Hz), 7.66 (d, 2H, J=9 Hz), 7.41 (d, 2H, J=8 Hz), 6.92 (d, 2H, J=8 Hz), 3.47 (s 1H), 3.0-3.3 (m, 4H), 1.82 (s, 1H), 1.54 (s, 6H), 1.4-1.6 (m, 4H) ppm.

### EXAMPLE 25

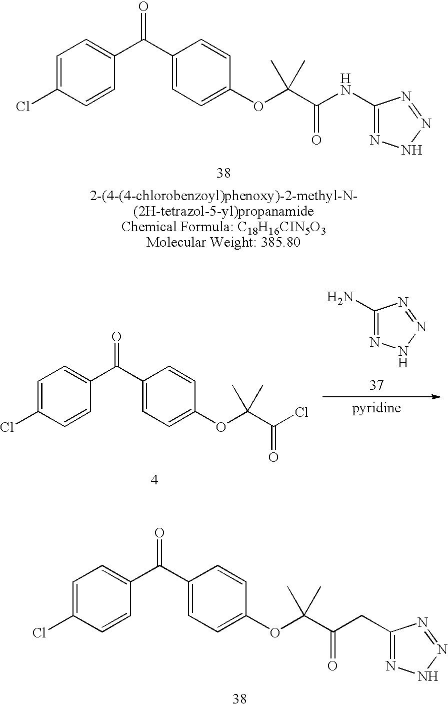

Step 1. Coupling of 5-aminotetrazole with fenofibric acid chloride (4) in pyridine gave 38 as white needles in 30% yield. ¹H-NMR (CD₃OD): δ 7.74 (d, 2H, J=8 Hz), 7.70 (d, 2H, J=9 Hz), 7.51 (d, 2H, J=8 Hz), 7.04 (d, 2H, J=8 Hz), 1.71 (s, 6H) ppm. See Hallinan, E. A.; Tsymbalov, S.; Dorn, C. R.; Pitzele, B. S.; Hansen, D. W.; Moore, W. M.; Jerome, G. M.; Connor, J. R.; Branson, L. F.; Widomski, D. L.; Zhang, Y.; Curie, M. G.; Manning, P. T. J. Med. Chem. 2002, 45, 1686-1689.

### EXAMPLE 26

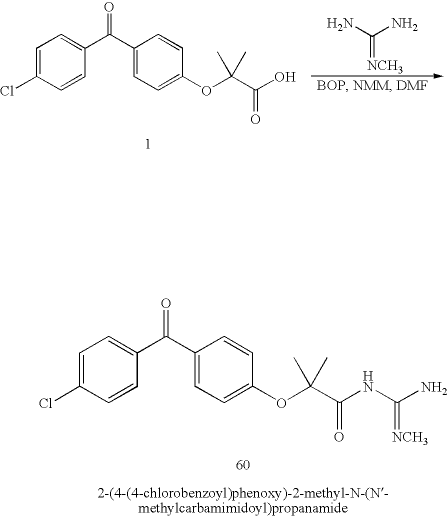

### EXAMPLE 27

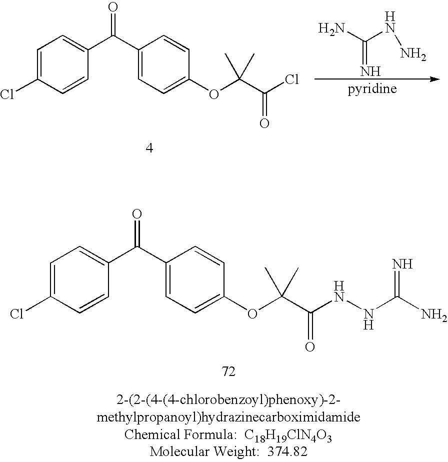

Coupling of aminoguanidine with fenofibric acid chloride (4) in pyridine gave 72 as a white solid in 38% yield. ¹H-NMR (CDCl₃): δ 11.0 (br s, 1H), 7.58 (d, 2H, J=8 Hz), 7.50 (d, 2H, J=9 Hz), 7.35 (d, 2H, J=8 Hz), 6.94 (d, 2H, J=8 Hz), 1.51 (s, 6H) ppm.

### EXAMPLE 28

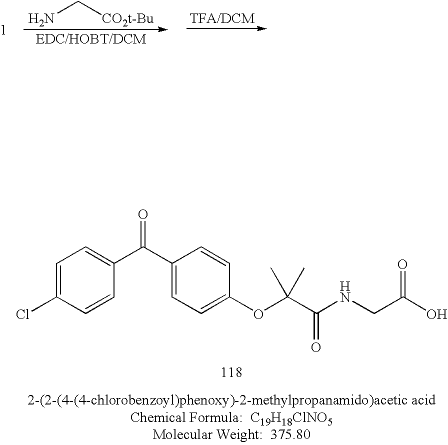

Step 1. Coupling of fenofibric acid (1) with EDC/HOBT in Dichloromethane followed by t-butylglycine gave t-butyl protected 118 in 79% yield after chromatography (ethyl acetate/hexane) ¹H-NMR (CDCl₃): δ 7.74 (d, 2H, J=9 Hz), 7.72 (d, 2H, J=9 Hz), 7.53 (d, 2H, J=8 Hz), 7.10 (d, 2H, J=8 Hz), 3.83 (s, 2H), 1.60 (s, 6H), 1.45 (s, 9H) ppm.

Step 2. Deprotection with trifluoroacetic acid in dichloromethane at room temperature (R.T.) gave 118 in 80% yield as white solid ¹H-NMR (CDCl₃): δ 7.75 (d, 2H, J=9 Hz), 7.73 (d, 2H, J=9 Hz), 7.53 (d, 2H, J=9 Hz), 7.12 (d, 2H, J=9 Hz), 3.77 (s, 2H), 1.60 (s, 6H) ppm.

### EXAMPLE 29

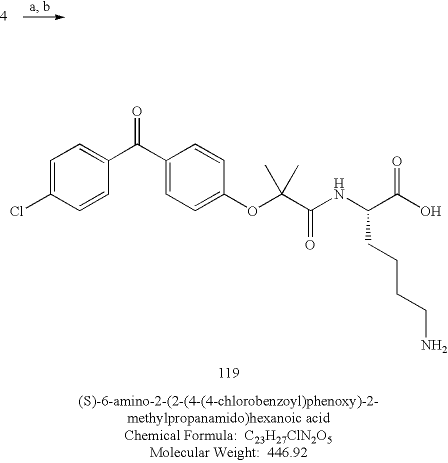

(a) treatment with (S)-2-amino-6-((2-(trimethylsilyl)ethoxy)carbonylamino)hexanoic acid, (from L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 30

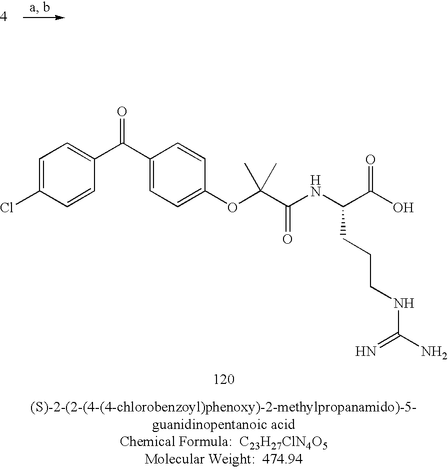

treatment with (S)-2-amino-5-guanidinopentanoic acid, (L-arginine) in DMF and N-methylmorpholine

### EXAMPLE 31

### A. Preparation of (E)-4-(3,5-dihydroxystyryl)phenyl 2-(4-(4-chlorobenzoyl)phenoxy)-2-methylpropanoate (10A) and (E)-3-hydroxy-5-(4-hydroxystyryl)phenyl 2-(4-(4-chlorobenzoyl)phenoxy)-2-methylpropanoate (12A).

10A and 12A, can be prepared from 4 by treatment with resveratrol ((E)-5-(4-hydroxystyryl)benzene-1,3-diol) (121) in the presence of cesium carbonate. This reaction gives a mixture of 10A and 12A that are separable by chromatography.

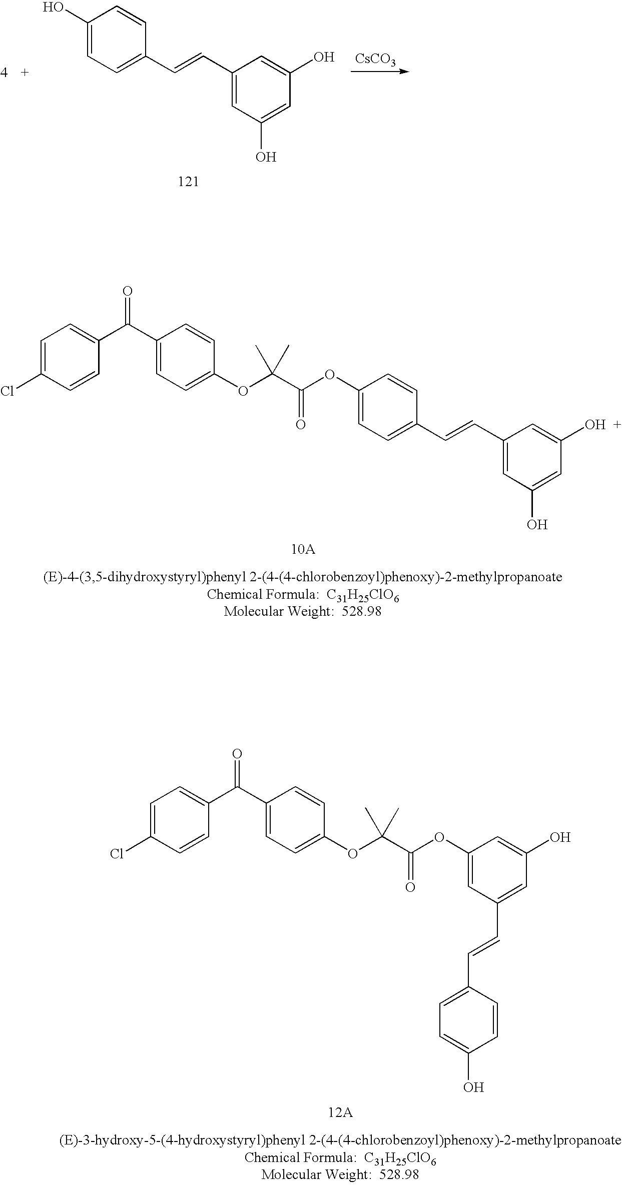

Compound 4 was coupled with 121 in THF with cesium carbonate to give 12A as well as 10A which were separated by chromatography (ethyl acetate/hexane). 12A ¹H-NMR (CDCl₃): δ 7.79 (d, 2H, J=9 Hz), 7.72 (d, 2H, J=9 Hz), 7.43 (d, 2H, J=9 Hz), 7.33 (d, 2H, J=9 Hz), 7.01 (d, 2H, J=9 Hz), 6.92 (d, 1H, J=16 Hz), 6.84-6.79 (m, 2H), 6.77 (d, 1H, J=16 Hz), 6.66-6.64 (m, 1H), 6.33-6.31 (m, 1H), 5.84 (s, 1H), 5.61 (dd, 1H, J=4 Hz, 2 Hz), 5.42 (s, 1H), 1.83 (s, 6H). 10A ¹H-NMR (CDCl₃): δ 7.80 (d, 2H, J=9 Hz), 7.73 (d, 2H, J=9 Hz), 7.46 (d, 4H, J=8 Hz), 6.88-7.04 (m, 6H), 6.56 (d, 2H, J=2 Hz), 6.28 (t, 1H, J=2 Hz), 4.85 (s, 2H), 1.84 (s, 6H).

Alternatively, (E)-4-(3,5-dihydroxystyryl)phenyl 2-(4-(4-chlorobenzoyl)phenoxy)-2-methylpropanoate (10A) can be prepared in the following manner.

### Step 1. Preparation of (E)-5-(4-(2-(4-(4-chlorobenzoyl)phenoxy)-2-methylpropanoyloxy)styryl)-1,3-phenylene diacetate (8A)

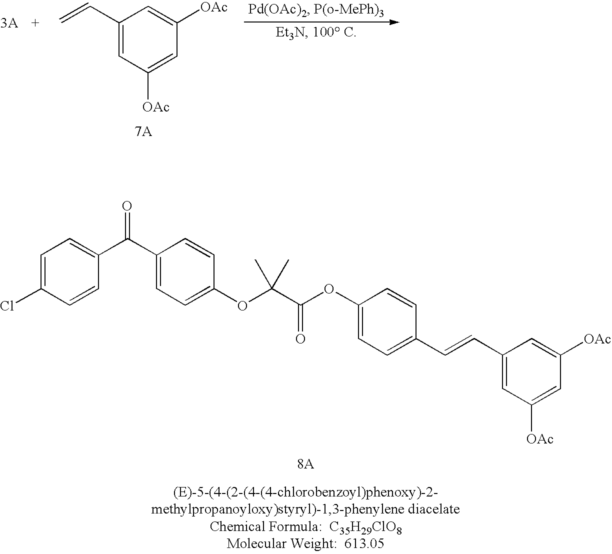

Iodoester 3A is coupled to styrene 7A in the presence of palladium (II) acetate, tri-o-tolylphosphine and triethylamine (Heck coupling conditions) to give (E)-5-(4-(2-(4-(4-chlorobenzoyl)phenoxy)-2-methylpropanoyloxy)styryl)-1,3-phenylene diacetate (8A) as off-white needles in 85% yield (mp: 112-113° C.).

### Step 2. Preparation of (E)-4-(3,5-dihydroxystyryl)phenyl 2-(4-(4-chlorobenzoyl)phenoxy)-2-methylpropanoate (10A)

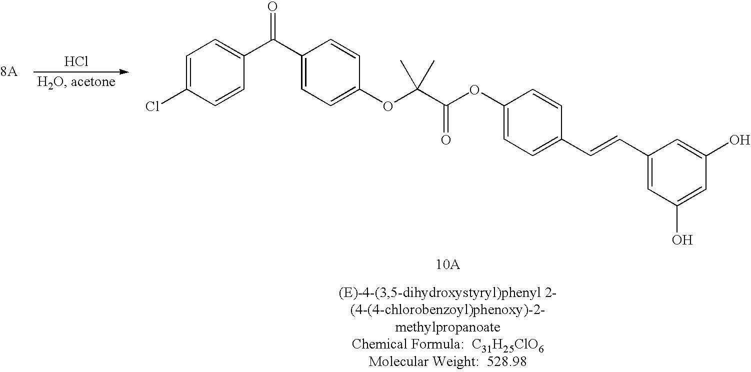

In the second step the acetate groups of 8A can be selectively removed by careful hydrolysis with HCl to give the bis-phenol (10A) as a light tan foam in 71% yield.

### B. 1. Preparation of (E)-4-(3,5-dimethoxystyryl)phenyl 2-(4-(4-chlorobenzoyl)phenoxy)-2-methylpropanoate (5A)

### Step 1. Preparation of 4-iodophenyl 2-(4-(4-chlorobenzoyl)phenoxy)-2-methylpropanoate (3A)

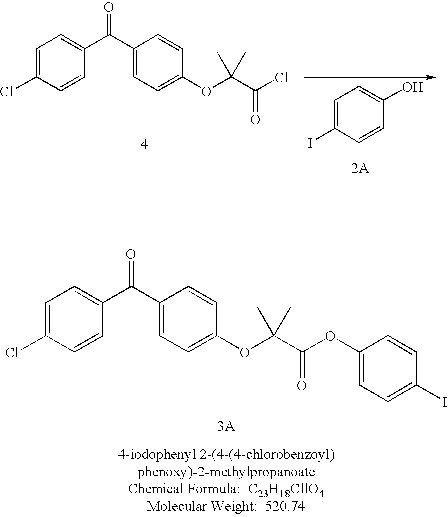

### Step 2. Preparation of (E)-4-(3,5-dimethoxystyryl)phenyl 2-(4-(4-chlorobenzoyl)phenoxy)-2-methylpropanoate (5A)

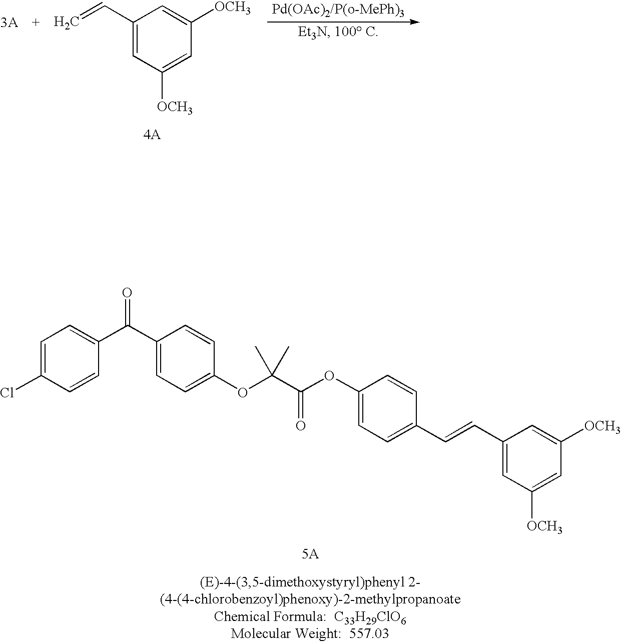

In the first step 2-(4-(4-chlorobenzoyl)phenoxy)-2-methylpropanoyl chloride (4) is condensed with 4-iodophenol (2) in the presence of cesium carbonate to provide 4-iodophenyl 2-(4-(4-chlorobenzoyl)phenoxy)-2-methylpropanoate (3A) as white prisms in 57% yield (mp 149-150° C. In the second step (3A) is reacted with 1,3-dimethoxy-5-vinylbenzene (4A) in the presence of palladium(II) acetate, tris-ortho-tolylphosphine and triethylamine at 100° C. to afford the Heck adduct (E)-4-(3,5-dimethoxystyryl)phenyl 2-(4-(4-chlorobenzoyl)phenoxy)-2-methylpropanoate (5A) as a white foam in 54% yield.

¹H-NMR (CDCl₃): δ 7.78 (d, 2H, J=9 Hz), 7.71 (d, 2H, J=9 Hz), 7.47 (d, 2H, J=8 Hz), 7.44 (d, 2H, J=8 Hz), 6.92-7.06 (m, 6H), 6.63 (d, 2H, J=2 Hz), 6.38 (t, 1H, J=2 Hz), 3.81 (s, 6H), 1.82 (s, 6H).

Alternatively, 5A was prepared directly from 4 by the condensation of pterostilbene ((E)-4-(3,5-dimethoxystyryl)phenol) (6A) in the presence of cesium carbonate in 84% yield.

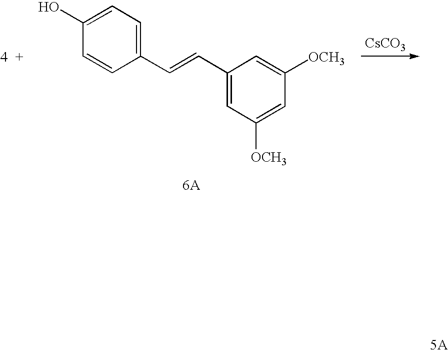

### REFERENCES

1. Farina, A.; Ferranti, C.; Marra, C. “An improved synthesis of resveratrol,” Natural Product Research 2006, 20, 247-252.
2. Guiso, M.; Marra, C.; Farina, A. “A new efficient resveratrol synthesis,” Tetrahedron Letters, 2002, 43, 597-598.

### EXAMPLE 32

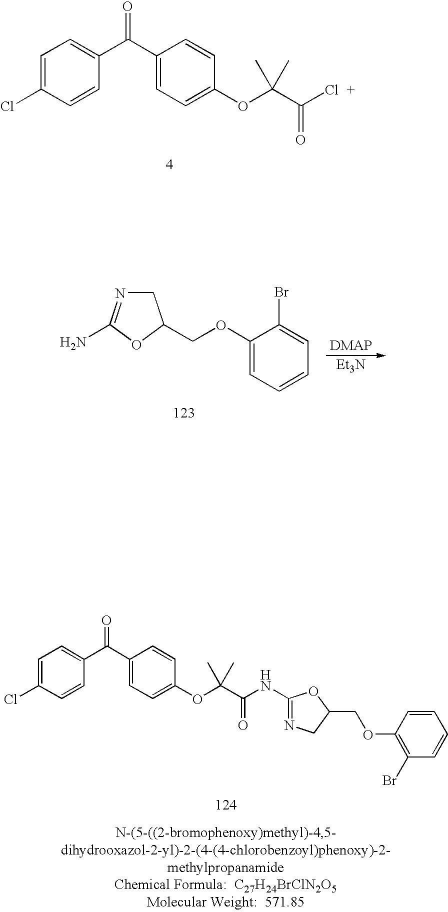

### EXAMPLE 33

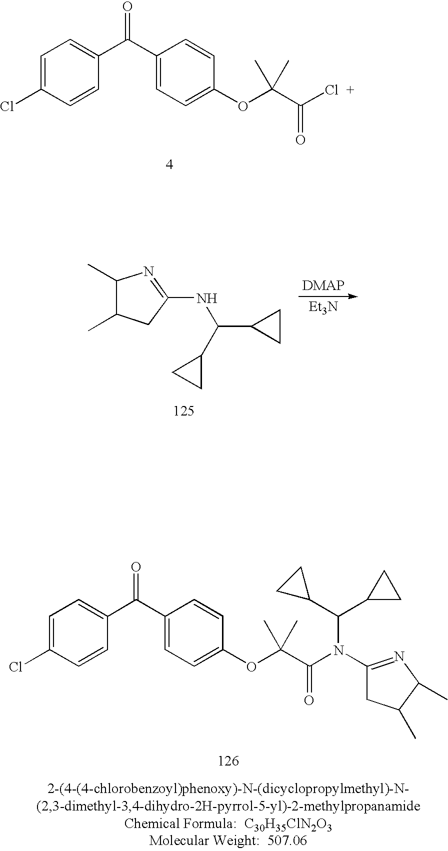

### EXAMPLE 34

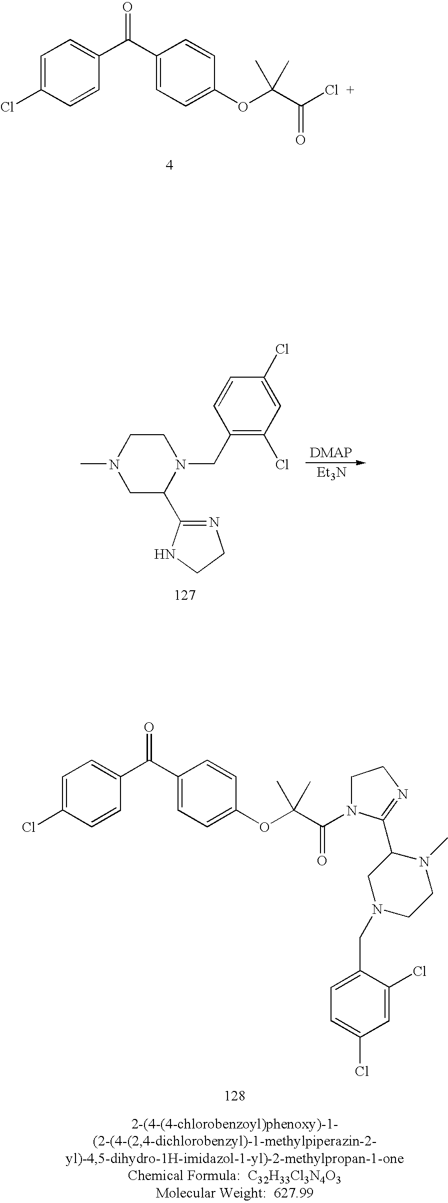

### EXAMPLE 35

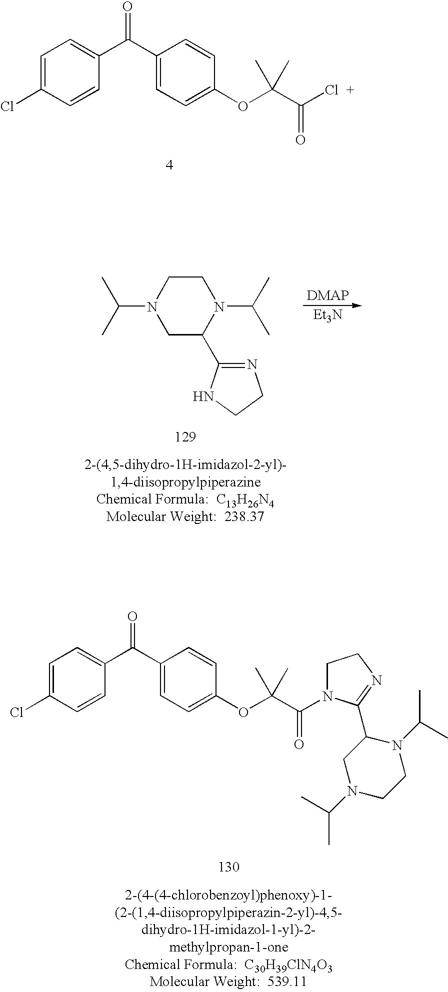

In other embodiments, the application provides compounds represented by the structure of Formula (II):

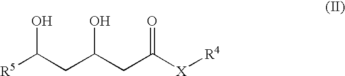

wherein

X is chosen from direct bond, O, NH, and an amino acid residue;

R⁴ is chosen from OH, NO, NO₂, an amino acid residue, a fibric acid residue, guanidine, tetrazolyl, agmatine, an amino-containing compound; lower alkyl terminating in ONO, (ONO₂)ₚ, or guanidine; a resveratrol residue; and an imidazoline receptor agonist residue;

wherein p is independently chosen from 1 to 3; and

R⁵ is chosen from a residue of a statin.

Methods of synthesis and specific experimental for representative compounds in the genus of Formula (II) include the following:

### EXAMPLE 36

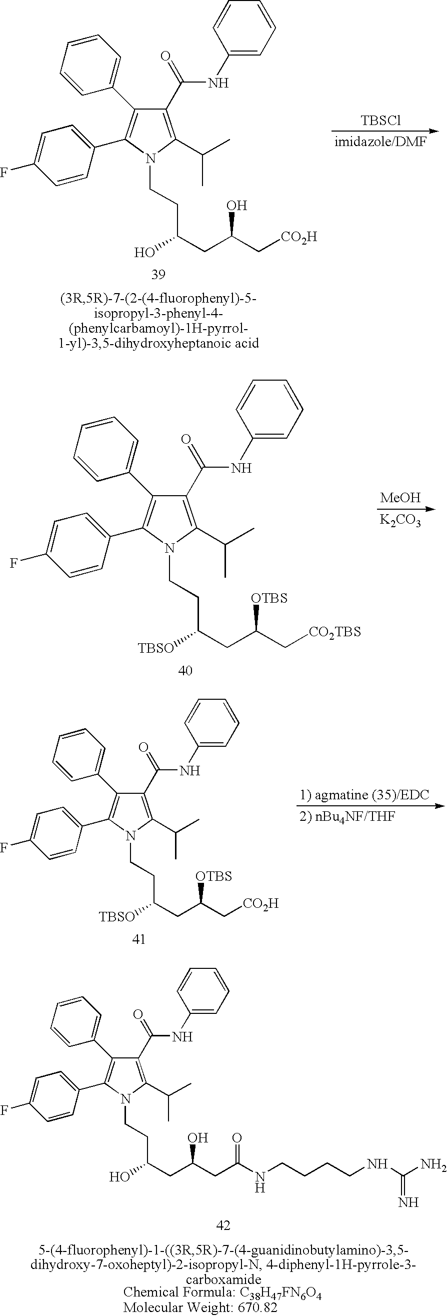

### EXAMPLE 37

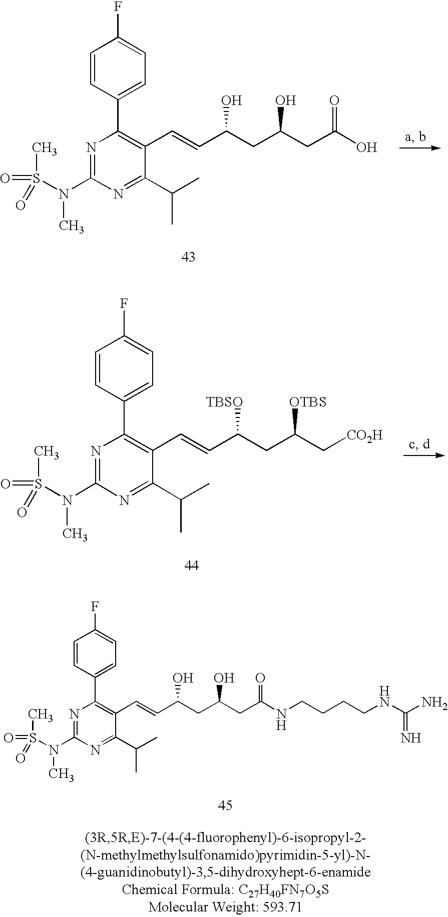

TBDMSCI, imidazole; (b) K₂CO₃; (c) agmatine (35), EDC; (d) nBu₄NF.

### EXAMPLE 38

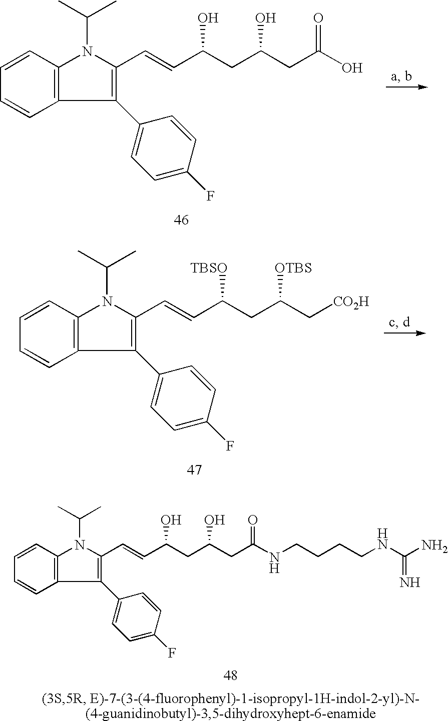

(a) TBDMSCl, imidazole; (b) MeOH, K₂CO₃; (c) agmatine (35), EDC; (d) nBu₄NF

### EXAMPLE 39

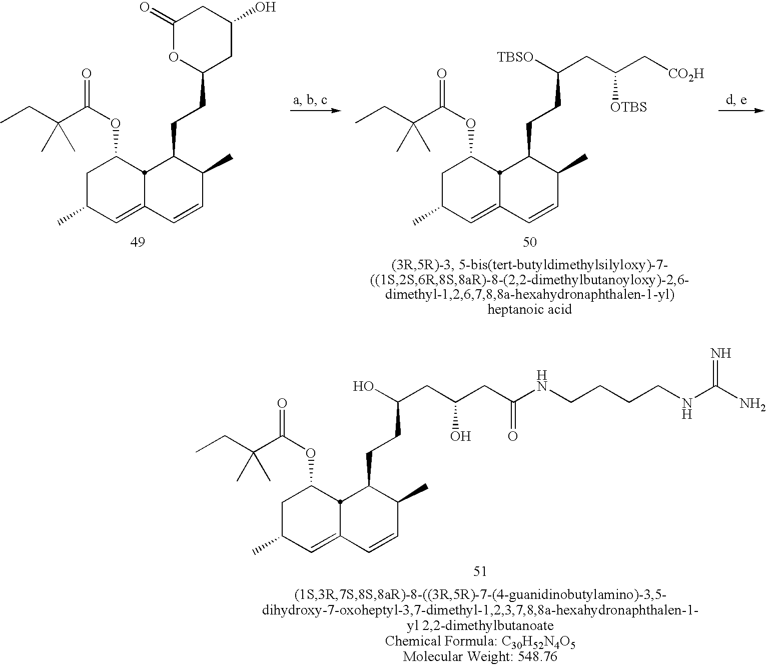

(a) LiOH, aq. THF; (b) TBDMSCl, imidazole; (c) MeOH, K₂CO₃; (d) agmatine (35), EDC; (e) nBU₄NF.

### EXAMPLE 40

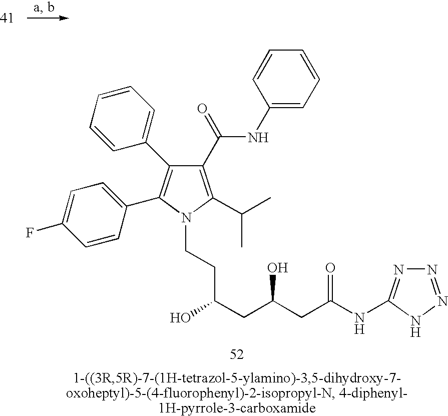

(a) BOP, NMM, DMF, 5-aminotetrazole (37); (b) nBu₄NF

### EXAMPLE 41

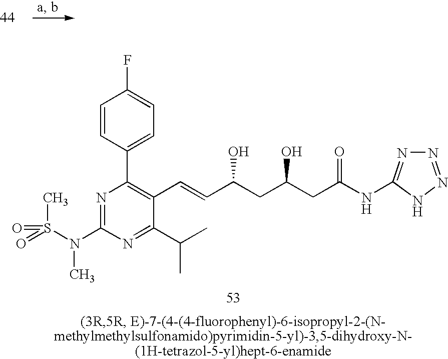

(a) BOP, NMM, DMF, 5-aminotetrazole (37); (b) nBu₄NF

### EXAMPLE 42

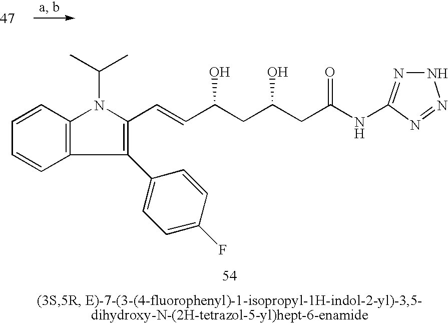

(a) BOP, NMM, DMF, 5-aminotetrazole (37); (b) nBu₄NF

### EXAMPLE 43

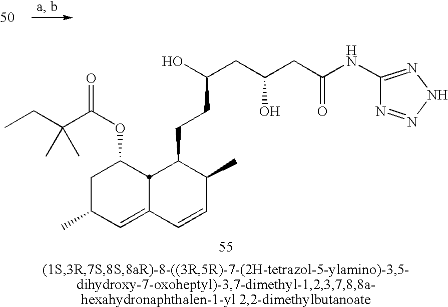

(a) BOP, NMM, DMF, 5-aminotetrazole (37); (b) nBu₄NF

### EXAMPLE 44

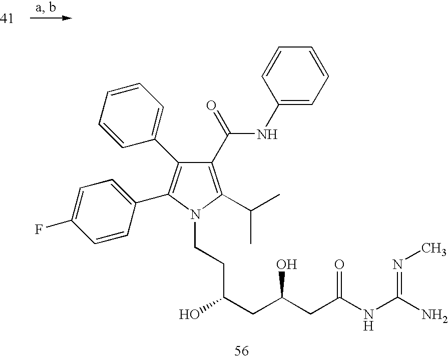

(a) BOP, NMM, DMF, methylguanidine; (b) nBu₄NF

### EXAMPLE 45

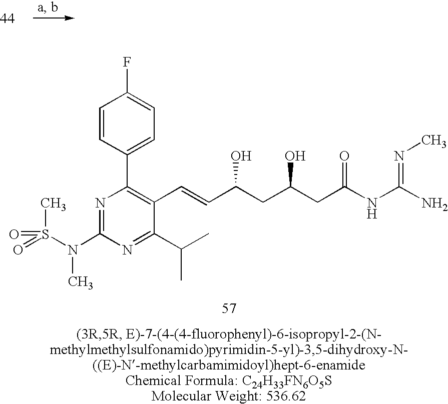

(a) BOP, NMM, DMF, methylguanidine; (b) nBu₄NF

### EXAMPLE 46

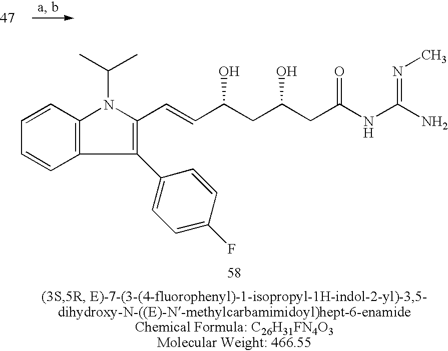

(a) BOP, NMM, DMF, methylguanidine; (b) nBu₄NF

### EXAMPLE 47

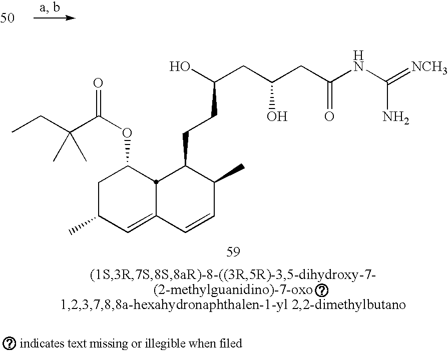

(a) BOP, NMM, DMF, methylguanidine; (b) nBu₄NF

### EXAMPLE 48

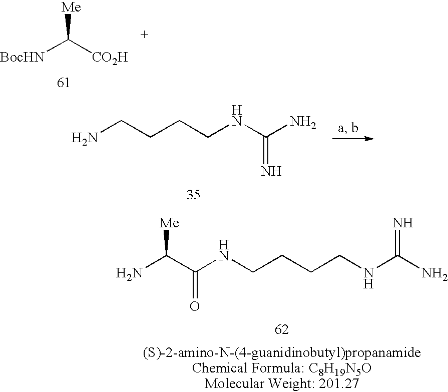

(a) EDC, DMF; (b) 4N HCl dioxane, then neutralize

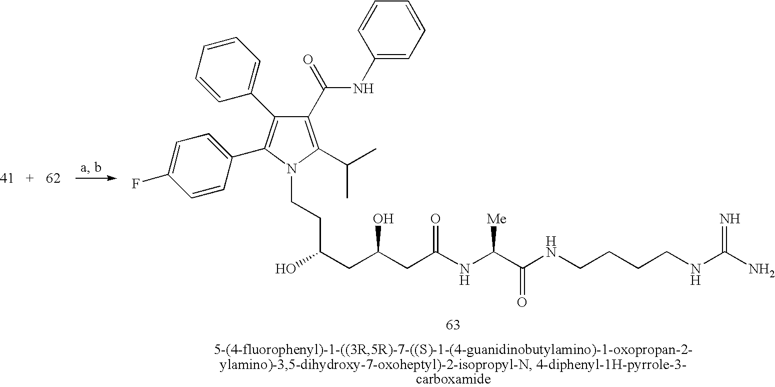

(a) (S)-2-amino-N-(4-guanidinobutyl)propanamide (62), EDC, DMF; (b) nBu₄NF

### EXAMPLE 49

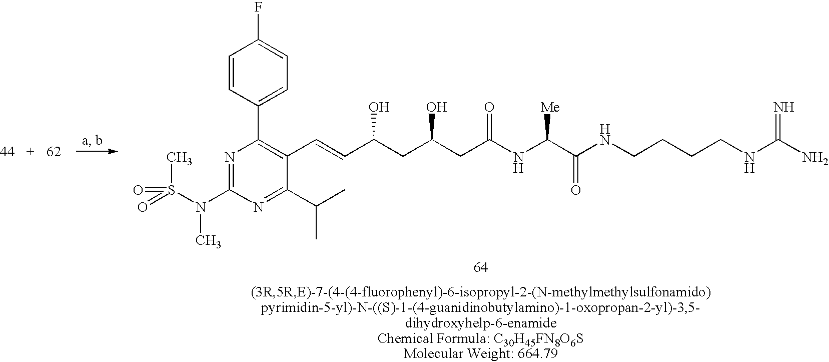

(a) (S)-2-amino-N-(4-guanidinobutyl)propanamide (62), EDC, DMF; (b) nBu₄NF

### EXAMPLE 50

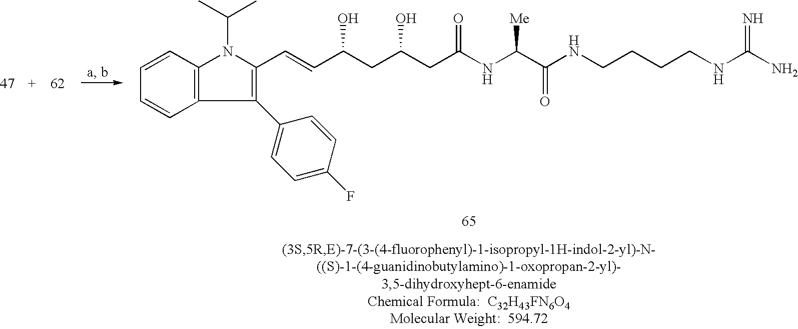

(a) (S)-2-amino-N-(4-guanidinobutyl)propanamide (62), EDC, DMF; (b) nBu₄NF

### EXAMPLE 51

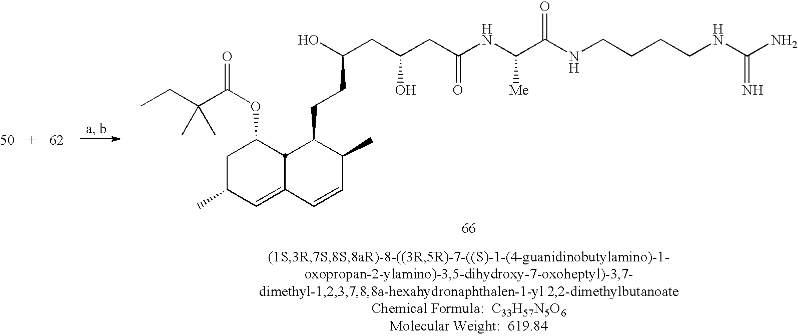

(a) (S)-2-amino-N-(4-guanidinobutyl)propanamide (62), EDC, DMF; (b) nBu₄NF

### EXAMPLE 52

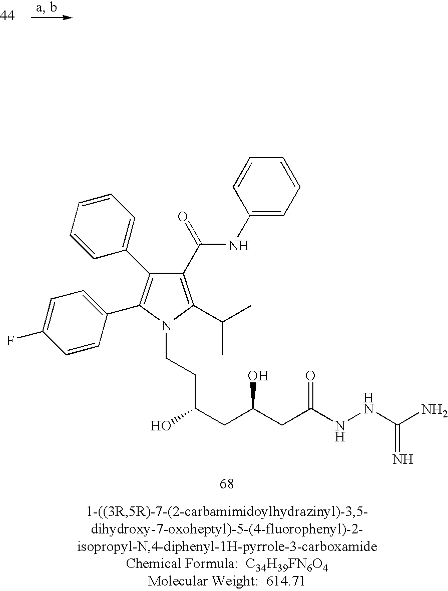

(a) aminoguanidine, EDC, DMF; (b) nBu₄NF

### EXAMPLE 53

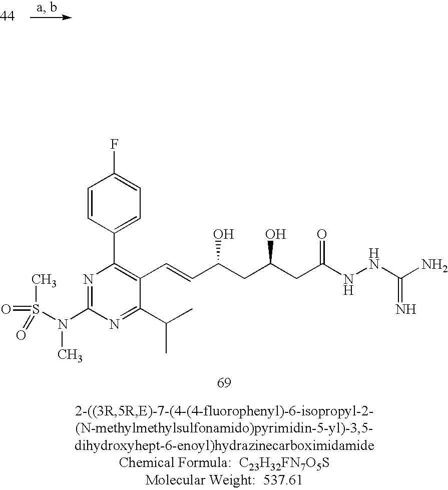

(a) aminoguanidine, EDC, DMF; (b) nBu₄NF

### EXAMPLE 54

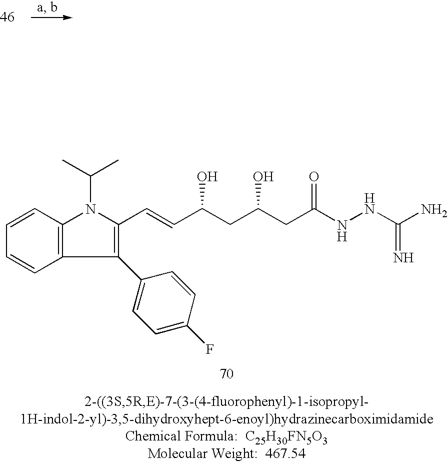

(a) aminoguanidine, EDC, DMF; (b) nBu₄NF

### EXAMPLE 55

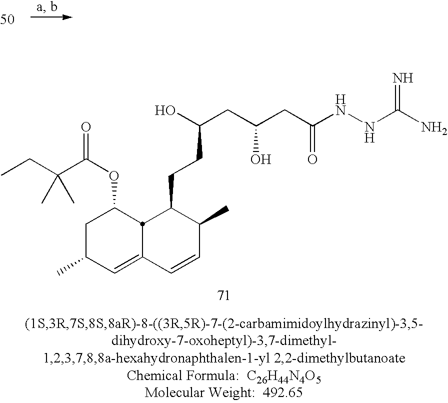

(a) aminoguanidine, EDC, DMF; (b) nBu₄NF

### EXAMPLE 56

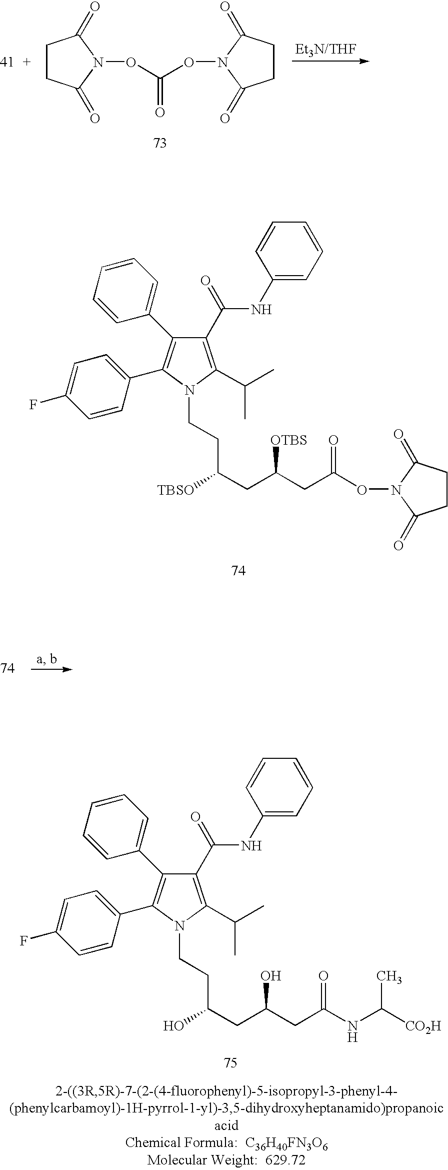

(a) treatment with 2-(trimethylsilyl)ethyl 2-aminopropanoate (D,L-alanine trimethylsilylethyl ester; from D,L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 57

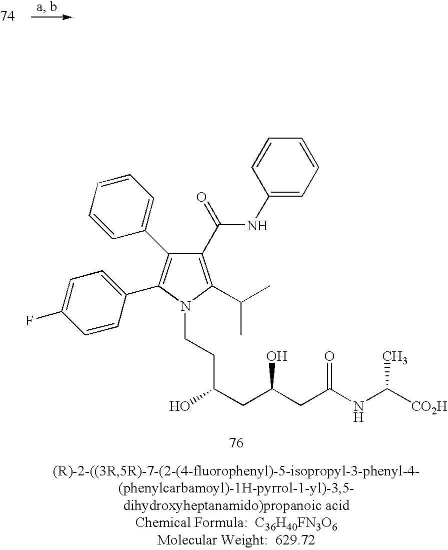

(a) treatment with (R)-2-(trimethylsilyl)ethyl 2-aminopropanoate (D-alanine trimethylsilylethyl ester; from D-alanine according to the method described in Bregman, Howard; Meggers, Eric. Organic Letters 2006, 8, 5465-5468) in DMF and N-methylmorpholine; (b) nBu₄NF

### EXAMPLE 58

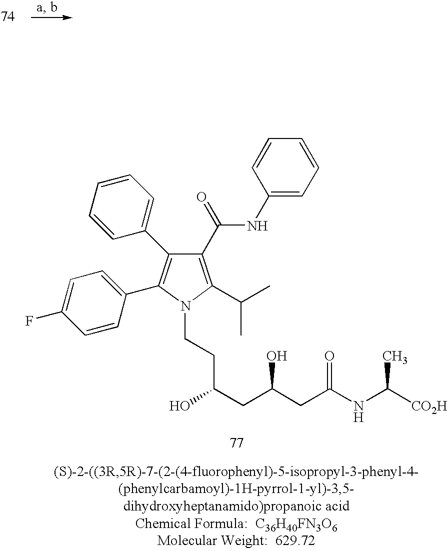

(a) treatment with (S)-2-(trimethylsilyl)ethyl 2-aminopropanoate (L-alanine trimethylsilylethyl ester; from L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 59

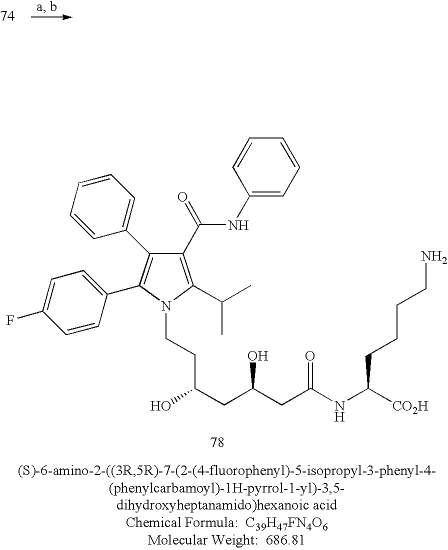

(a) treatment with (S)-2-amino-6-((2-(trimethylsilyl)ethoxy)carbonylamino)hexanoic acid, (from L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 60

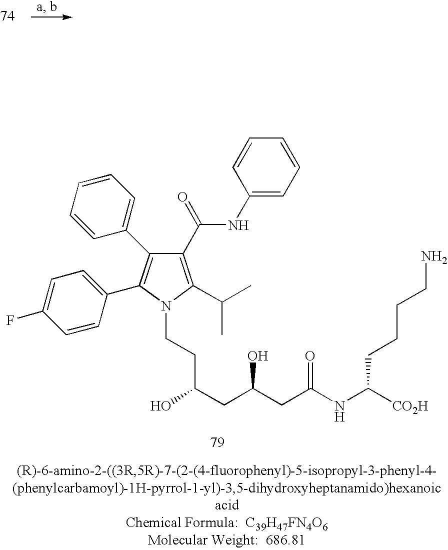

(a) treatment with (R)-2-amino-6-((2-(trimethylsilyl)ethoxy)carbonylamino)hexanoic acid, (from D-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 61

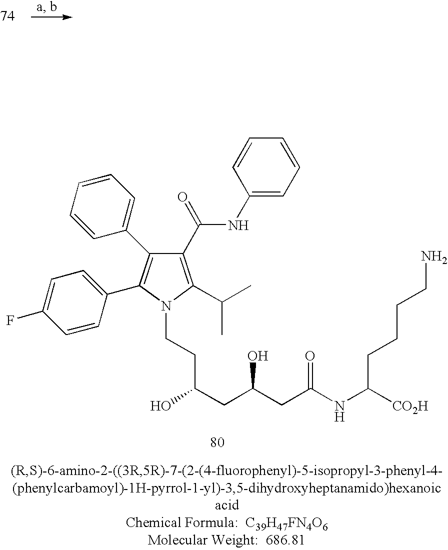

(a) treatment with (R,S)-2-amino-6-((2-(trimethylsilyl)ethoxy)carbonylamino)hexanoic acid, (from D,L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 62

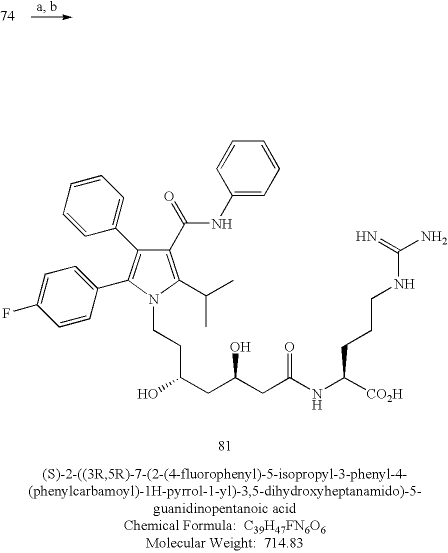

(a) treatment with (S)-2-amino-5-guanidinopentanoic acid, (L-arginine) in DMF and N-methylmorpholine; (b) nBu₄NF

### EXAMPLE 63

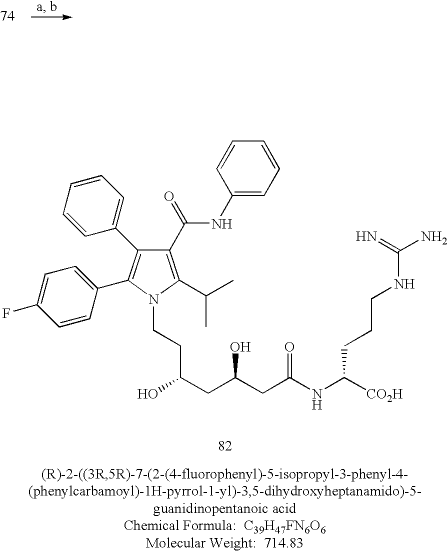

(a) treatment with (R)-2-amino-5-guanidinopentanoic acid, (D-arginine) in DMF and N-methylmorpholine; (b) nBu₄NF

### EXAMPLE 64

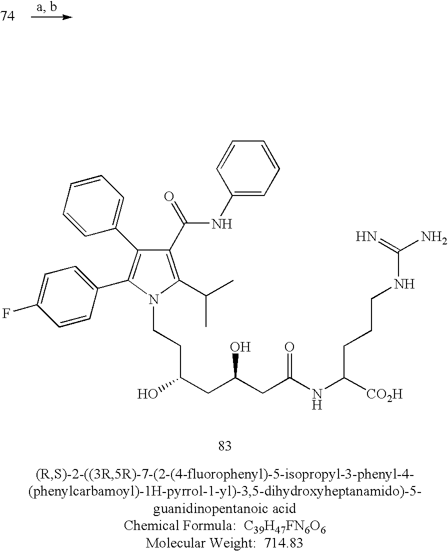

(a) treatment with (R,S)-2-amino-5-guanidinopentanoic acid, (D,L-arginine) in DMF and N-methylmorpholine; (b) nBu₄NF

### EXAMPLE 65

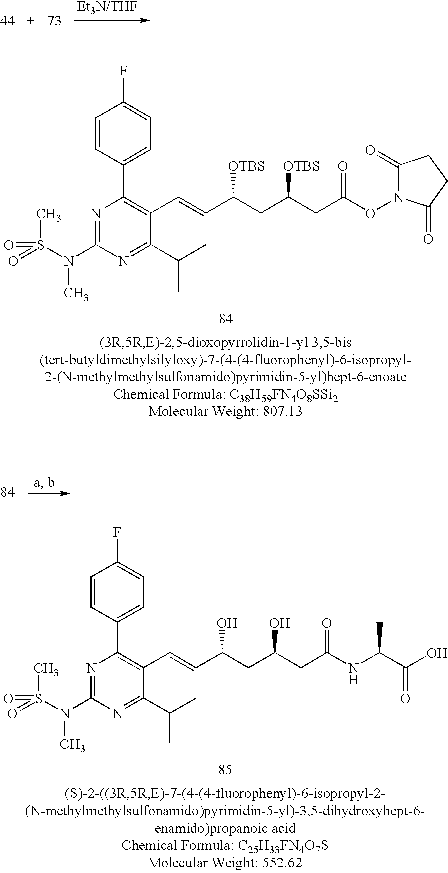

(a) treatment with (S)-2-(trimethylsilyl)ethyl 2-aminopropanoate (L-alanine trimethylsilylethyl ester; from L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 66

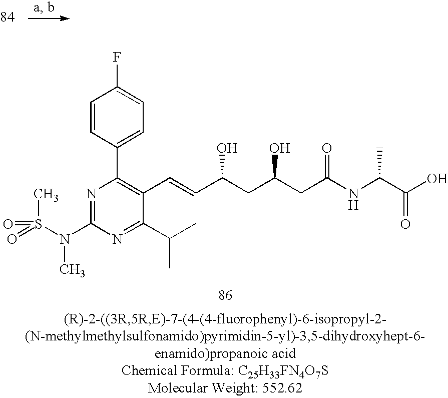

(a) treatment with (R)-2-(trimethylsilyl)ethyl 2-aminopropanoate (D-alanine trimethylsilylethyl ester; from D-alanine according to the method described in Bregman, Howard; Meggers, Eric. Organic Letters 2006, 8, 5465-5468) in DMF and N-methylmorpholine; (b) nBu₄NF

### EXAMPLE 67

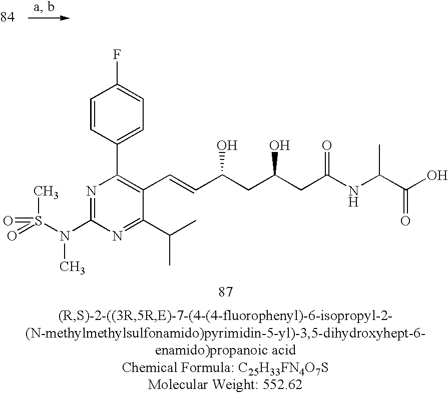

(a) treatment with 2-(trimethylsilyl)ethyl 2-aminopropanoate (D,L-alanine trimethylsilylethyl ester; from D,L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 68

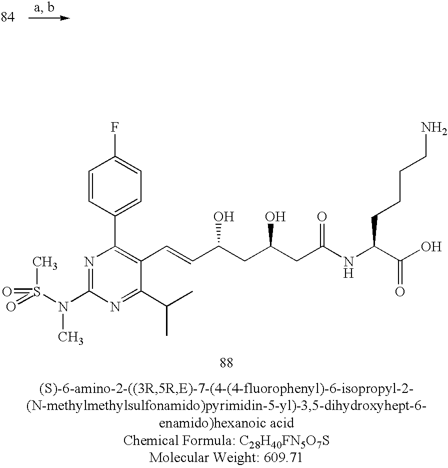

(a) treatment with (S)-2-amino-6-((2-(trimethylsilyl)ethoxy)carbonylamino)hexanoic acid, (from L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 69

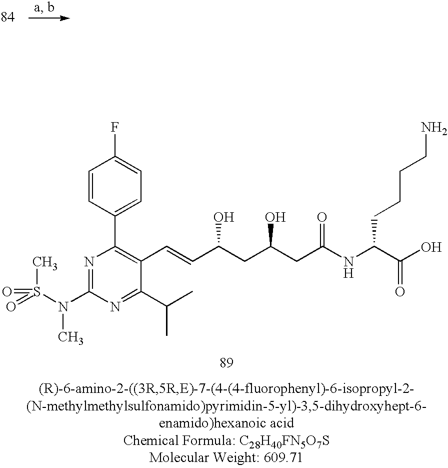

(a) treatment with (R)-2-amino-6-((2-(trimethylsilyl)ethoxy)carbonylamino)hexanoic acid, (from D-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 70

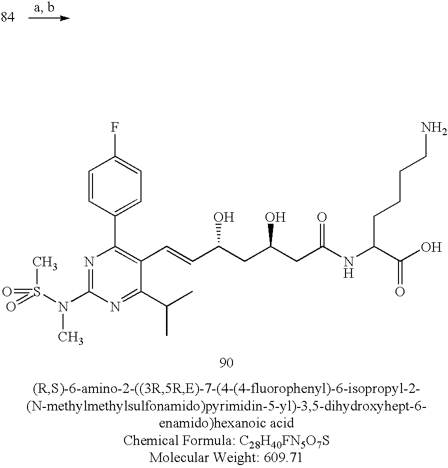

(a) treatment with (R,S)-2-amino-6-((2-(trimethylsilyl)ethoxy)carbonylamino)hexanoic acid, (from D,L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 71

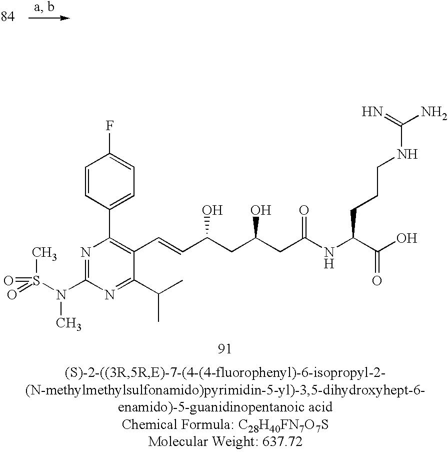

(a) treatment with (S)-2-amino-5-guanidinopentanoic acid, (L-arginine) in DMF and N-methylmorpholine; (b) nBu₄NF

### EXAMPLE 72

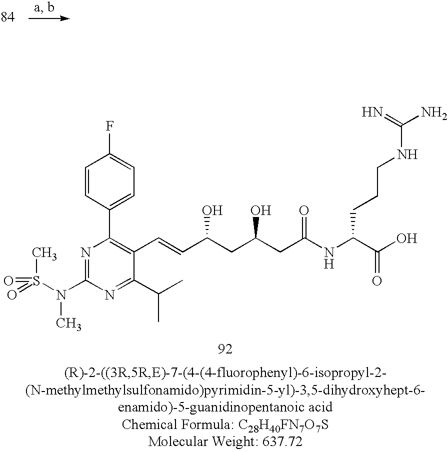

(a) treatment with (R)-2-amino-5-guanidinopentanoic acid, (D-arginine) in DMF and N-methylmorpholine; (b) nBu₄NF

### EXAMPLE 73

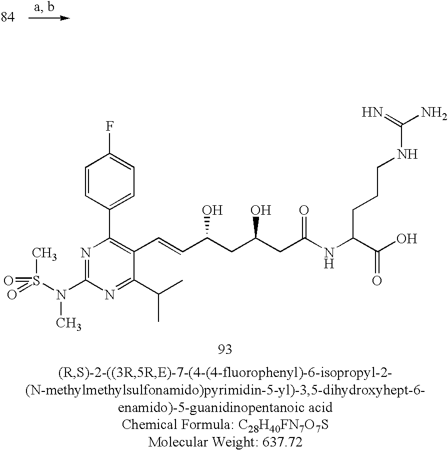

(a) treatment with (R,S)-2-amino-5-guanidinopentanoic acid, (D,L-arginine) in DMF and N-methylmorpholine; (b) nBu₄NF

### EXAMPLE 74

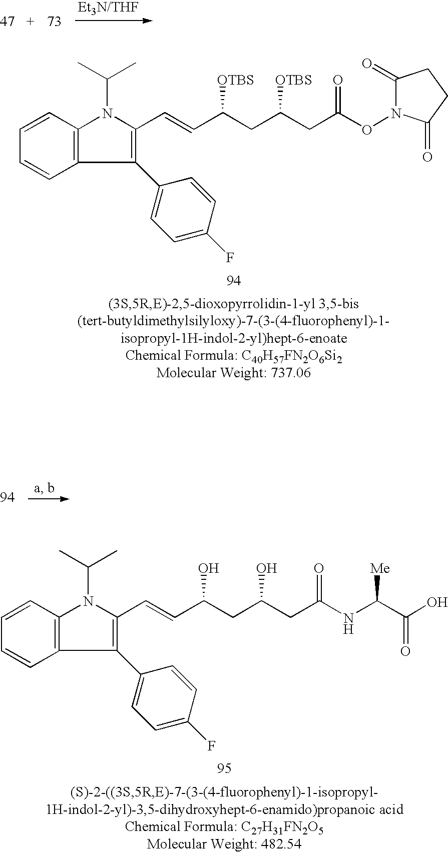

(a) treatment with (S)-2-(trimethylsilyl)ethyl 2-aminopropanoate (L-alanine trimethylsilylethyl ester; from L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 75

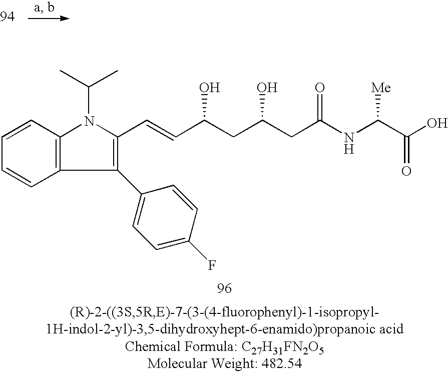

(a) treatment with (R)-2-(trimethylsilyl)ethyl 2-aminopropanoate (D-alanine trimethylsilylethyl ester; from D-alanine according to the method described in Bregman, Howard; Meggers, Eric. Organic Letters 2006, 8, 5465-5468) in DMF and N-methylmorpholine; (b) nBu₄NF

### EXAMPLE 76

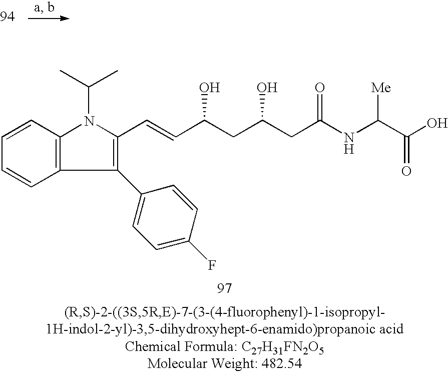

(a) treatment with 2-(trimethylsilyl)ethyl 2-aminopropanoate (D,L-alanine trimethylsilylethyl ester; from D,L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 77

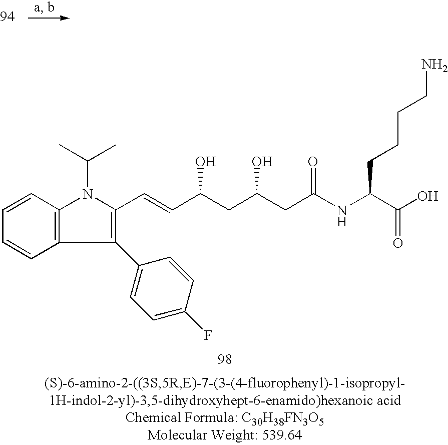

(a) treatment with (S)-2-amino-6-((2-(trimethylsilyl)ethoxy)carbonylamino)hexanoic acid, (from L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 78

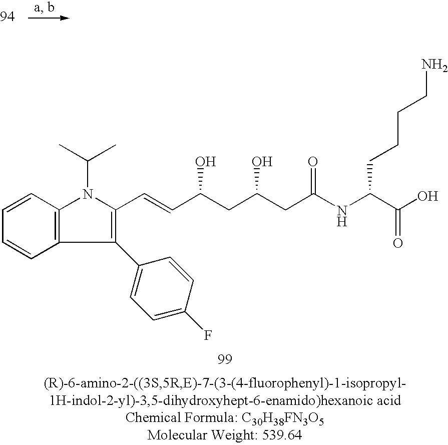

(a) treatment with (R)-2-amino-6-((2-(trimethylsilyl)ethoxy)carbonylamino)hexanoic acid, (from D-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 79

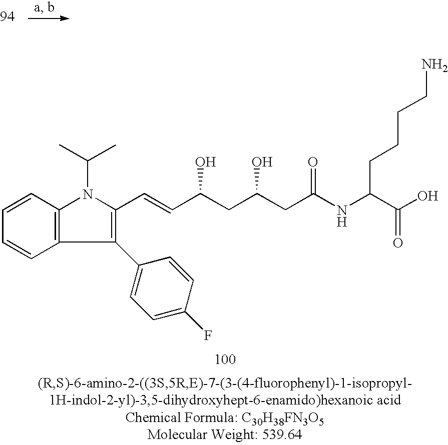

(a) treatment with (R,S)-2-amino-6-((2-(trimethylsilyl)ethoxy)carbonylamino)hexanoic acid, (from D,L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 80

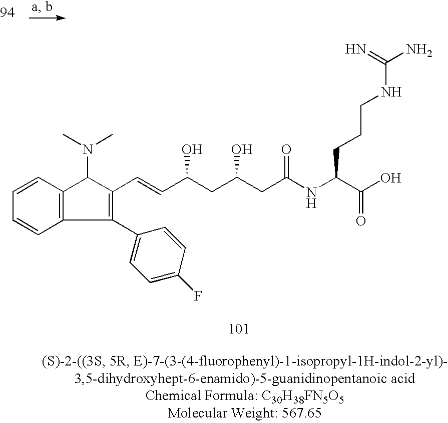

(a) treatment with (S)-2-amino-5-guanidinopentanoic acid, (L-arginine) in DMF and N-methylmorpholine; (b) nBu₄NF

### EXAMPLE 81

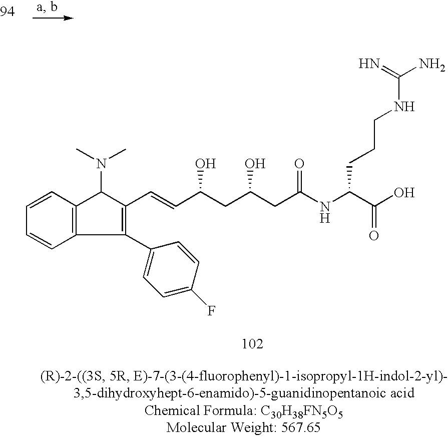

(a) treatment with (R)-2-amino-5-guanidinopentanoic acid, (D-arginine) in DMF and N-methylmorpholine; (b) nBu₄NF

### EXAMPLE 82

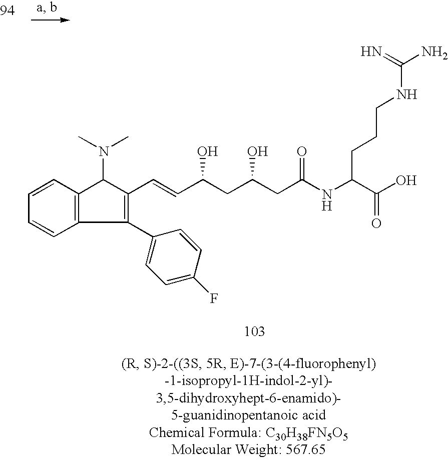

(a) treatment with (R,S)-2-amino-5-guanidinopentanoic acid, (D,L-arginine) in DMF and N-methylmorpholine; (b) nBu₄NF

### EXAMPLE 83

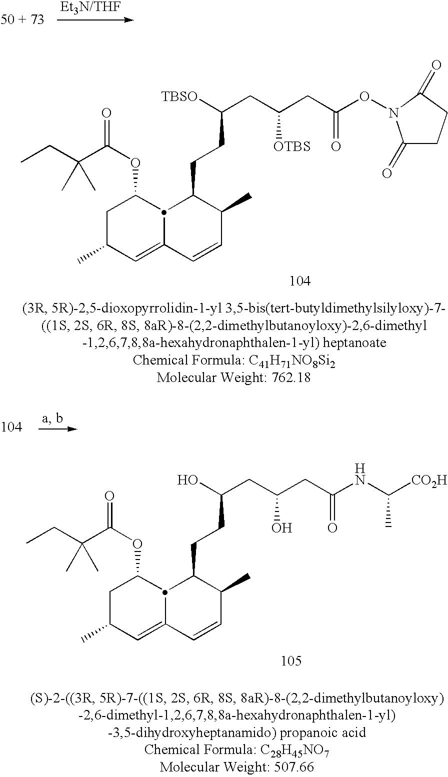

(a) treatment with (S)-2-(trimethylsilyl)ethyl 2-aminopropanoate (L-alanine trimethylsilylethyl ester; from L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 84

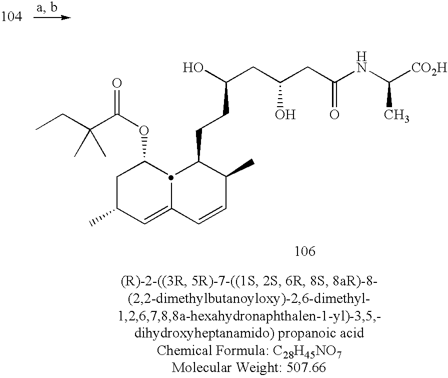

(a) treatment with (R)-2-(trimethylsilyl)ethyl 2-aminopropanoate (D-alanine trimethylsilylethyl ester; from D-alanine according to the method described in Bregman, Howard; Meggers, Eric. Organic Letters 2006, 8, 5465-5468) in DMF and N-methylmorpholine; (b) nBu₄NF

### EXAMPLE 85

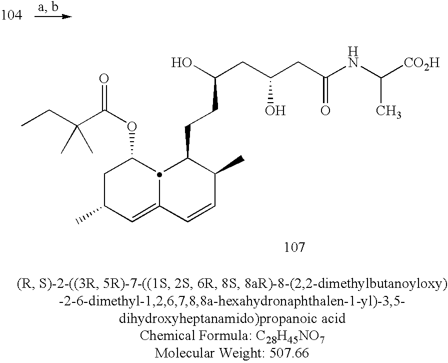

(a) treatment with 2-(trimethylsilyl)ethyl 2-aminopropanoate (D,L-alanine trimethylsilylethyl ester; from D,L-alanine according to the method described in Godfrey, J. D., Jr.; Gordon, E. M.; Von Langen, D.; Engebrecht, J.; Pluscec, Jelka. Journal of Organic Chemistry 1986, 51, 3073-3075) in DMF and N-methylmorpholine; (b) nBu₄NF

### EXAMPLE 86

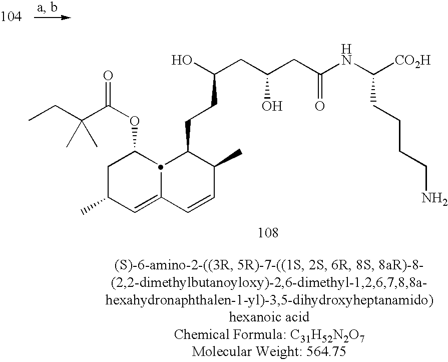

(a) treatment with (S)-2-amino-6-((2-(trimethylsilyl)ethoxy)carbonylamino)hexanoic acid, (from L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 87

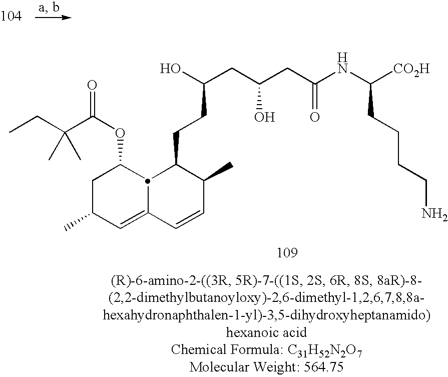

(a) treatment with (R)-2-amino-6-((2-(trimethylsilyl)ethoxy)carbonylamino)hexanoic acid, (from D-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 88

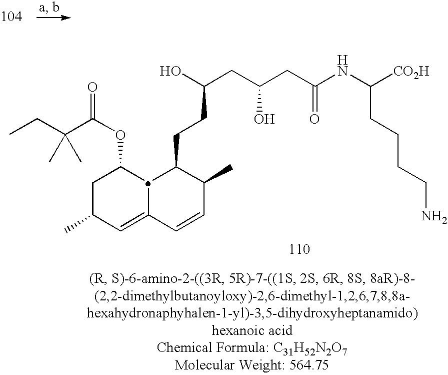

(a) treatment with (R,S)-2-amino-6-((2-(trimethylsilyl)ethoxy)carbonylamino)hexanoic acid, (from D,L-lysine according to the method described in Rosowsky, Andre; Wright, Joel E. Journal of Organic Chemistry 1983, 48, 1539-1541) in DMF and N-methylmorpholine; (b) nBu₄NF

### EXAMPLE 89

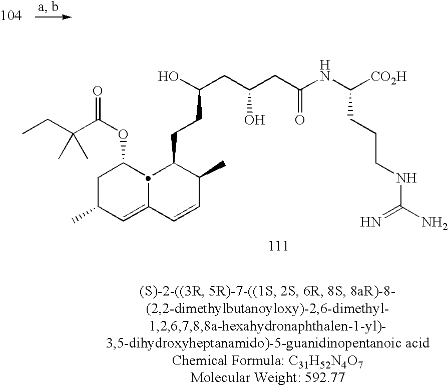

(a) treatment with (S)-2-amino-5-guanidinopentanoic acid, (L-arginine) in DMF and N-methylmorpholine; (b) nBu₄NF

### EXAMPLE 90

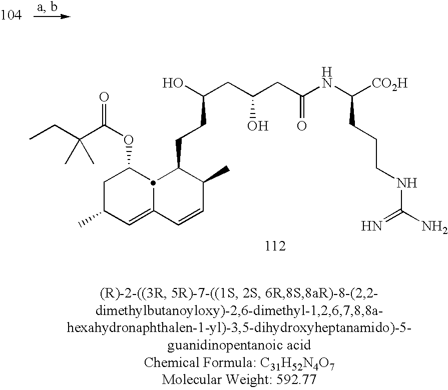

(a) treatment with (R)-2-amino-5-guanidinopentanoic acid, (D-arginine) in DMF and N-methylmorpholine; (b) nBu₄NF

### EXAMPLE 91

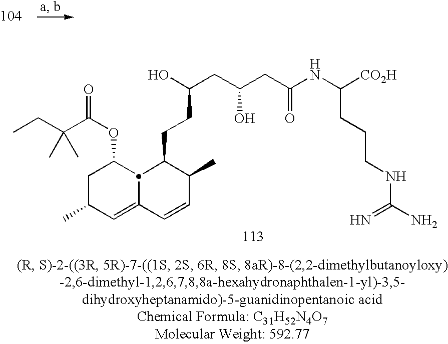

(a) treatment with (R,S)-2-amino-5-guanidinopentanoic acid, (D,L-arginine) in DMF and N-methylmorpholine; (b) nBu₄NF

### EXAMPLE 92

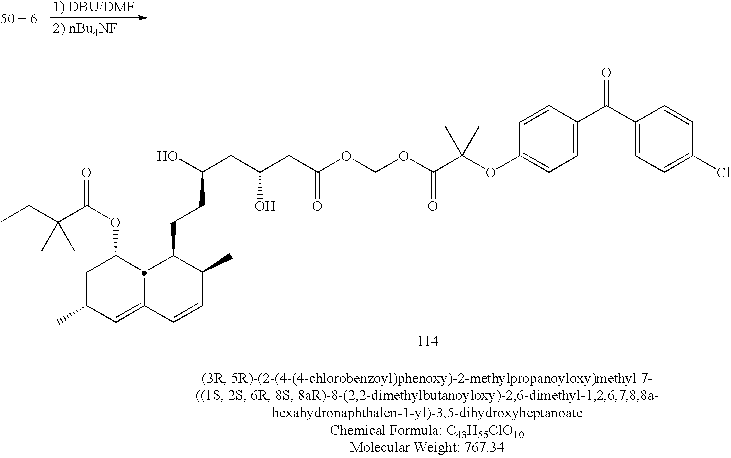

### EXAMPLE 93

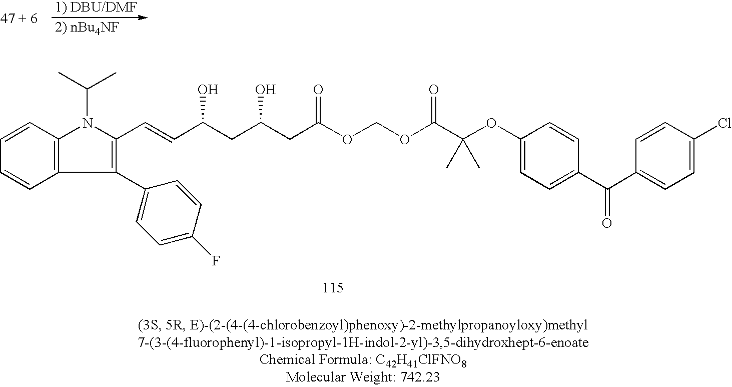

### EXAMPLE 94

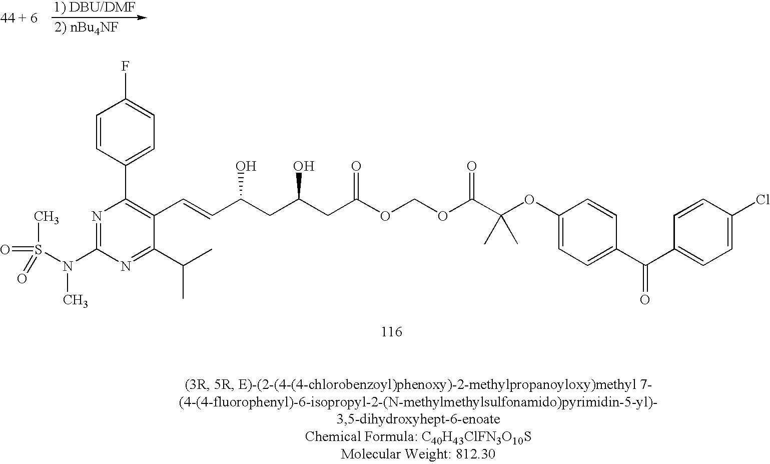

### EXAMPLE 95

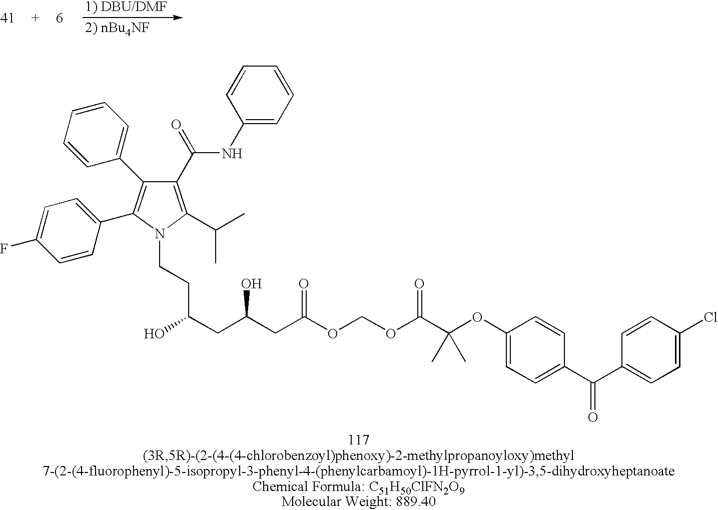

### EXAMPLE 96

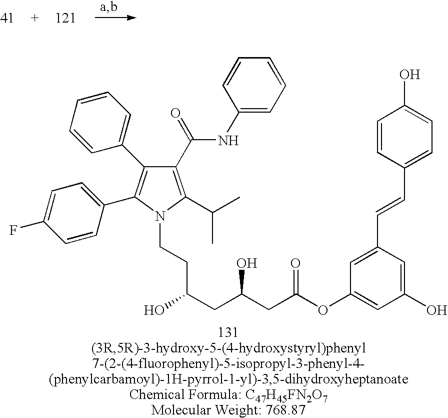

(a) DCC, DMAP; (b) nBu₄NF

### EXAMPLE 97

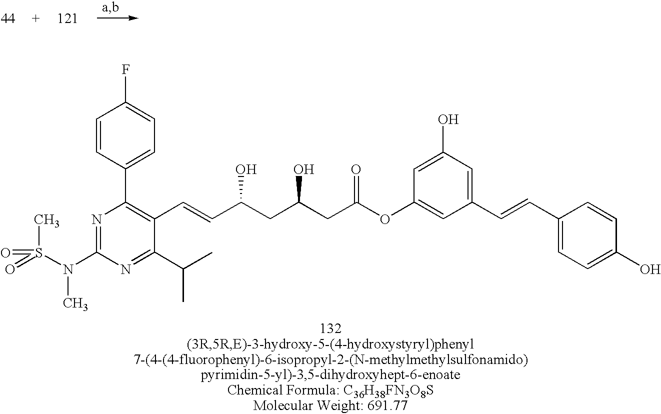

(a) DCC, DMAP; (b) nBu₄NF

### EXAMPLE 98

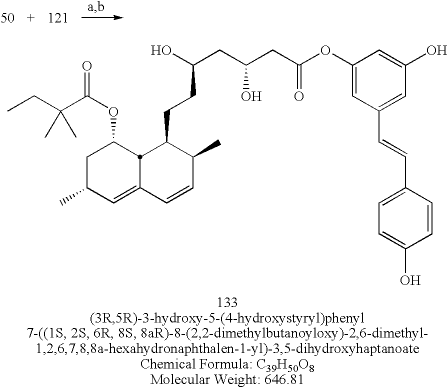

(a) DCC, DMAP; (b) nBu₄NF

### EXAMPLE 99

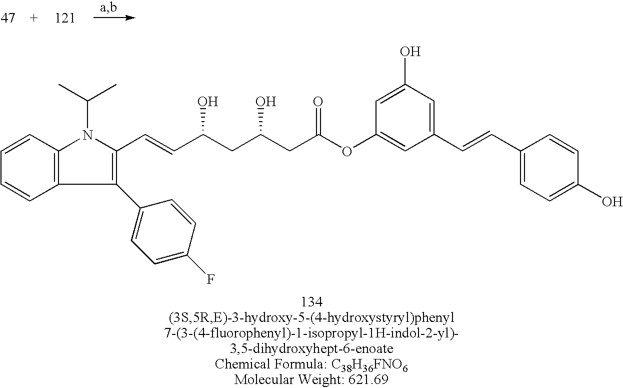

(a) DCC, DMAP; (b) nBu₄NF

### EXAMPLE 100

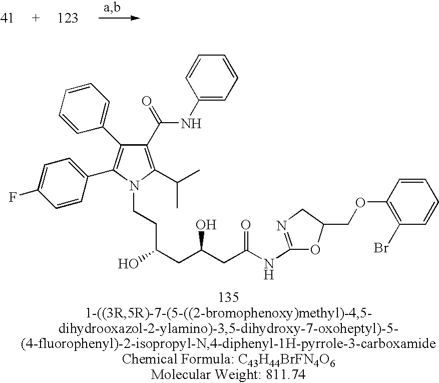

(a) DCC, DMAP; (b) nBu₄NF

### EXAMPLE 101

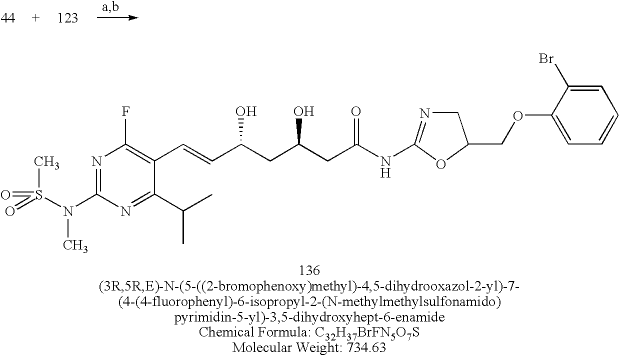

(a) DCC, DMAP; (b) nBu₄NF

### EXAMPLE 102

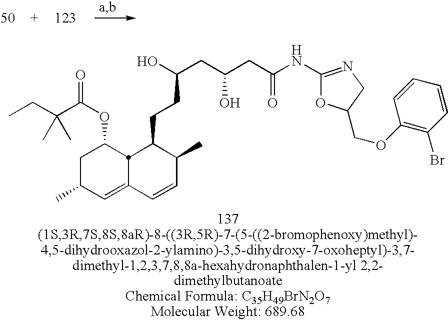

(a) DCC, DMAP; (b) nBu₄NF

### EXAMPLE 103

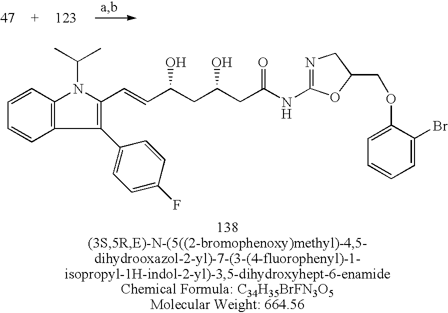

(a) DCC, DMAP; (b) nBu₄NF

### EXAMPLE 104

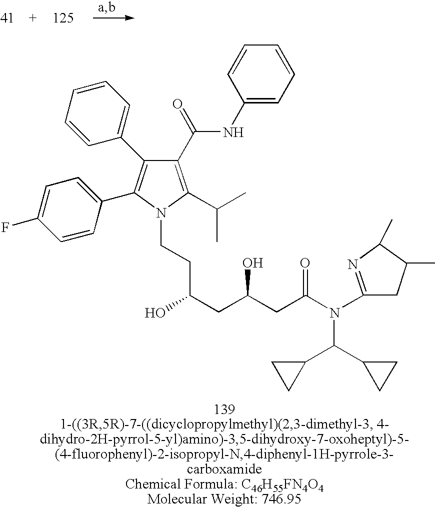

(a) DCC, DMAP; (b) nBu₄NF

### EXAMPLE 105

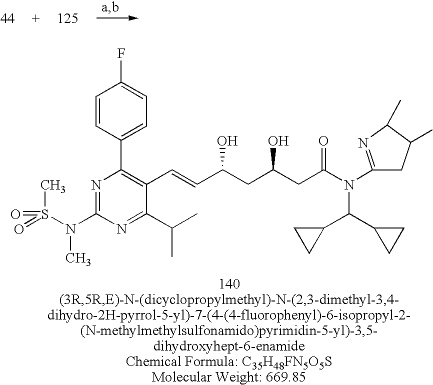

(a) DCC, DMAP; (b) nBu₄NF

### EXAMPLE 106

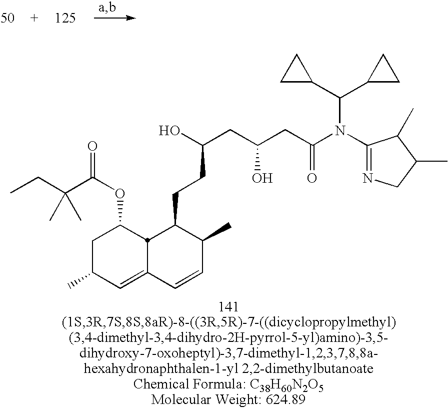

(a) DCC, DMAP; (b) nBu₄NF

### EXAMPLE 107

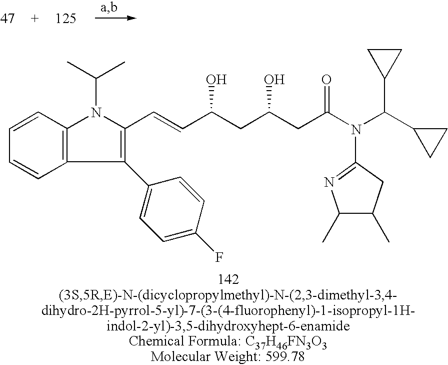

(a) DCC, DMAP; (b) nBu₄NF

### EXAMPLE 108

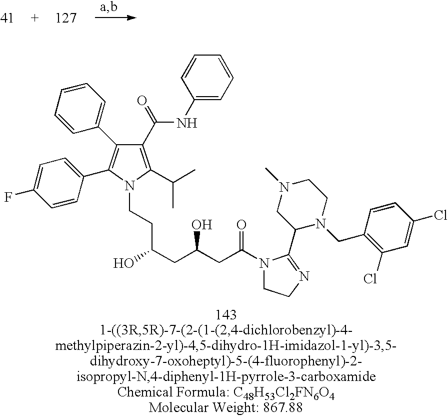

(a) DCC, DMAP; (b) nBu₄NF

### EXAMPLE 109

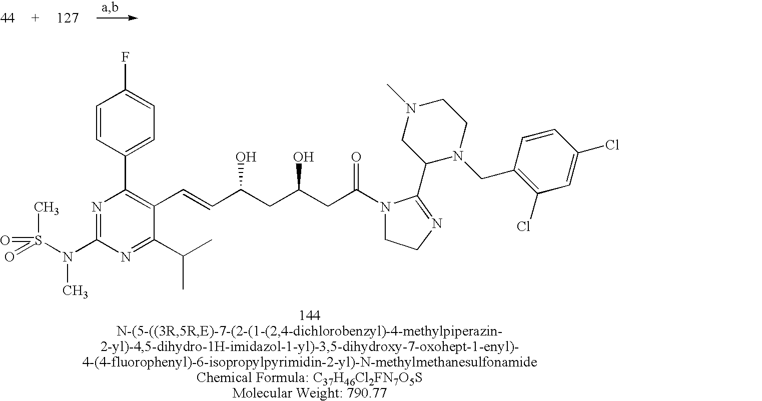

(a) DCC, DMAP; (b) nBu₄NF

### EXAMPLE 110

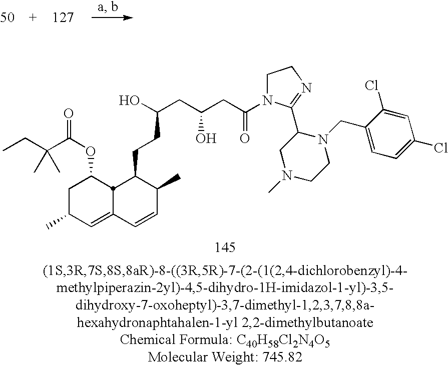

(a) DCC, DMAP; (b) nBu₄NF

### EXAMPLE 111

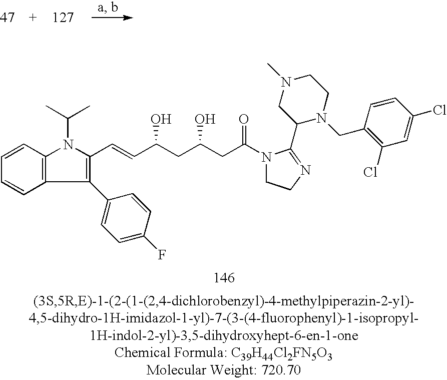

(a) DCC, DMAP; (b) nBu₄NF

### EXAMPLE 112

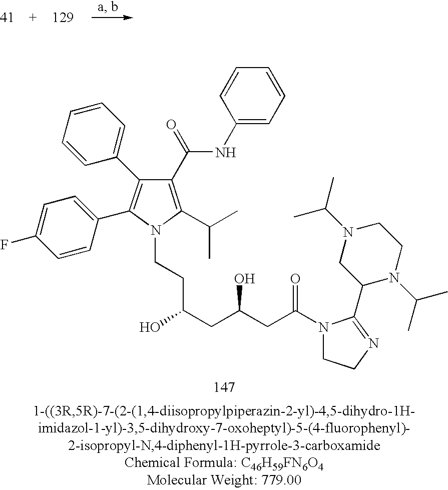

(a) DCC, DMAP; (b) nBu₄NF

### EXAMPLE 113

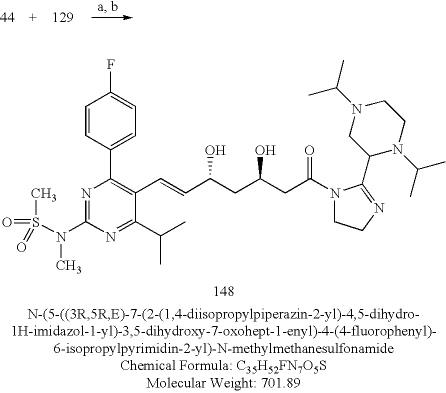

(a) DCC, DMAP; (b) nBu₄NF

### EXAMPLE 114

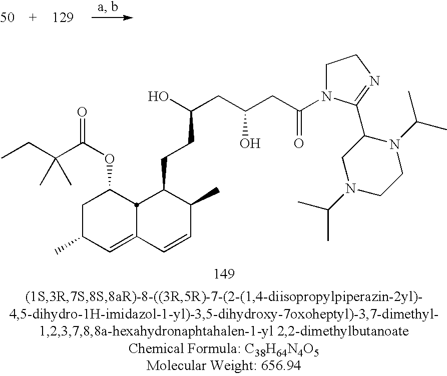

(a) DCC, DMAP; (b) nBu₄NF

### EXAMPLE 115

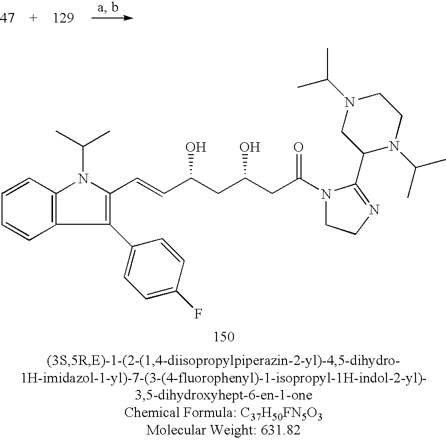

(a) DCC, DMAP; (b) nBu₄NF

In still other embodiments, the present disclosure provides compounds comprising a fibric acid or statin derivative in the form of a salt, wherein a fibric acid or statin residue is provided as a cation or anion, and another molecule is provided as a corresponding counterion. In certain embodiments, the counterion includes, but is not limited to, a NOS substrate or an amino-tetrazole compound. Although those of ordinary skill in the art would recognize that any fibric acid or statin compound can be used, along with counterions other than those disclosed immediately below, methods of synthesis and specific experimental for representative compounds include the following:

### EXAMPLE 116

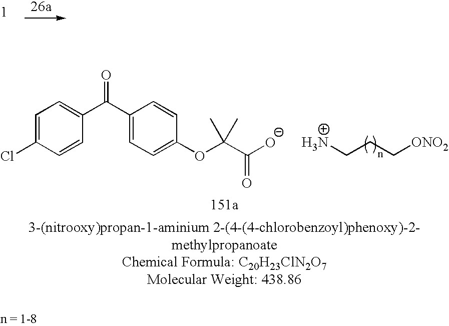

### EXAMPLE 117

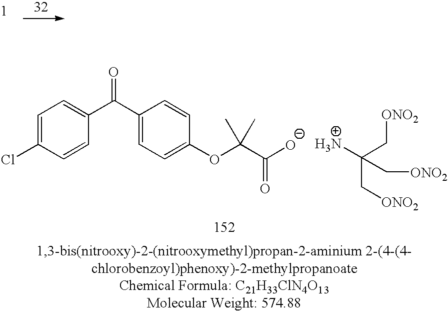

### EXAMPLE 118

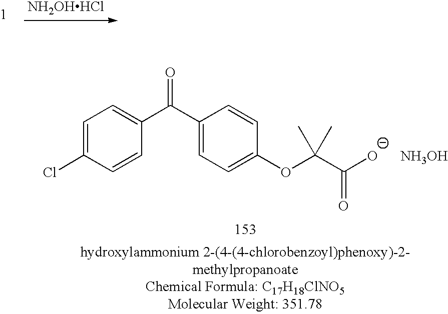

### EXAMPLE 119

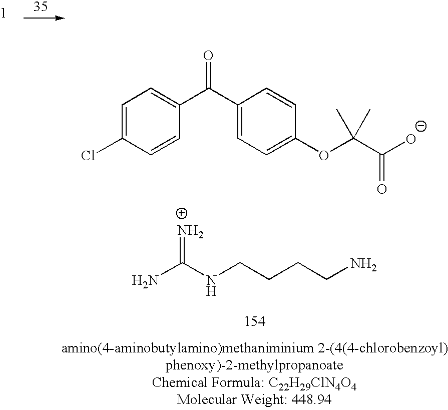

### EXAMPLE 120

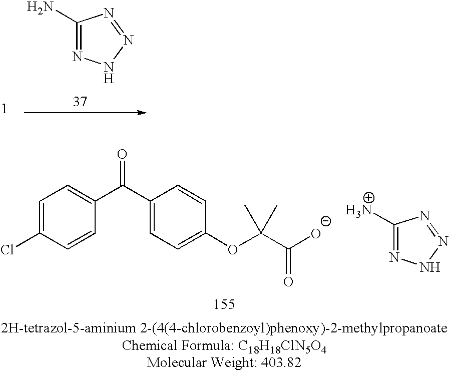

### EXAMPLE 121

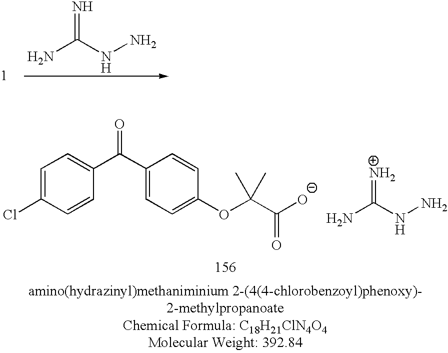

### EXAMPLE 122

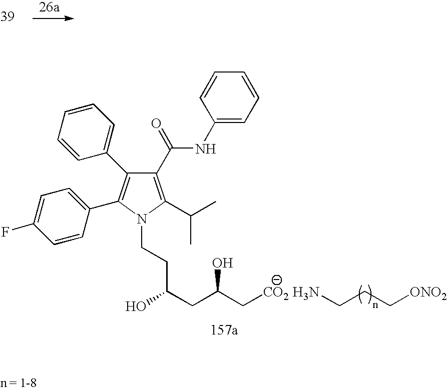

### EXAMPLE 123

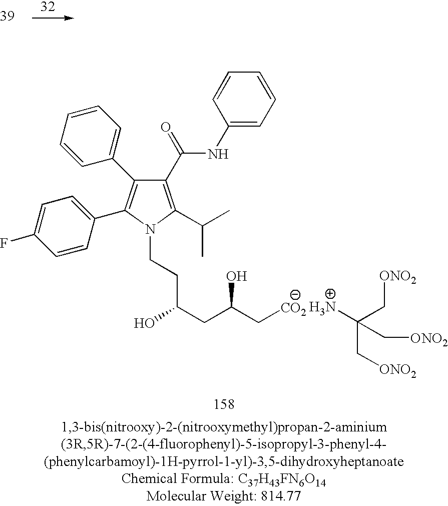

### EXAMPLE 124

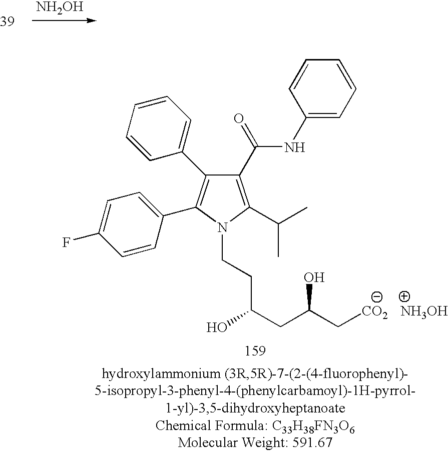

### EXAMPLE 125

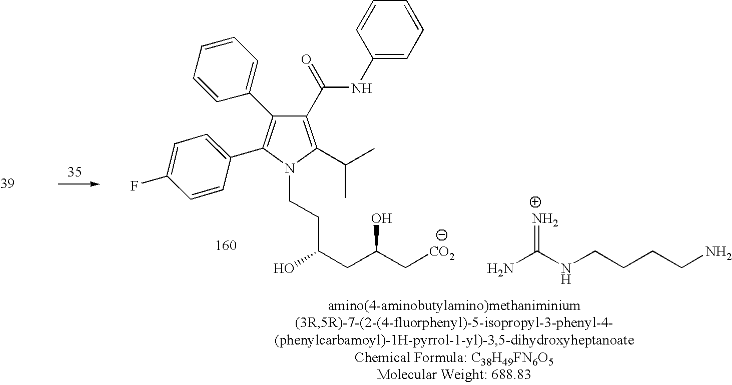

### EXAMPLE 126

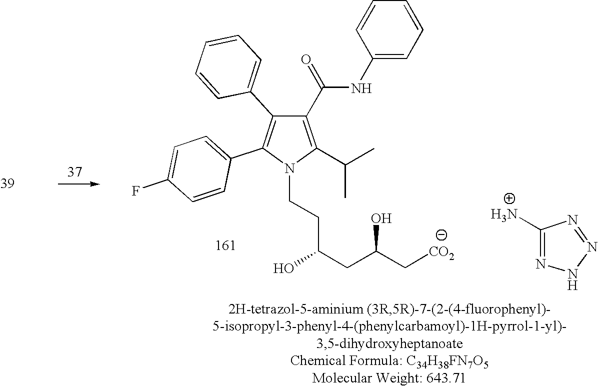

### EXAMPLE 127

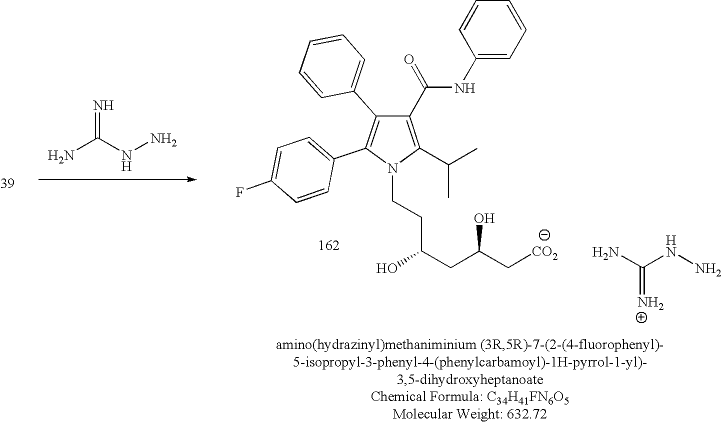

### EXAMPLE 128

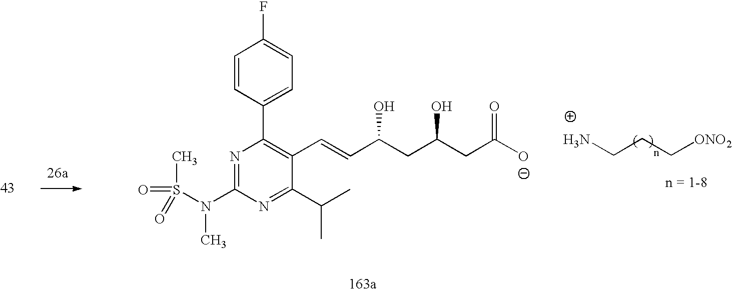

### EXAMPLE 129

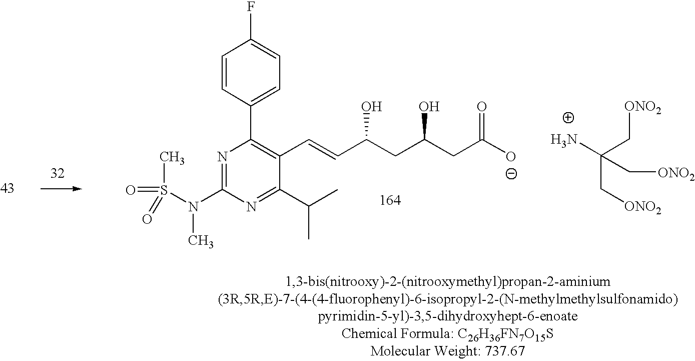

### EXAMPLE 130

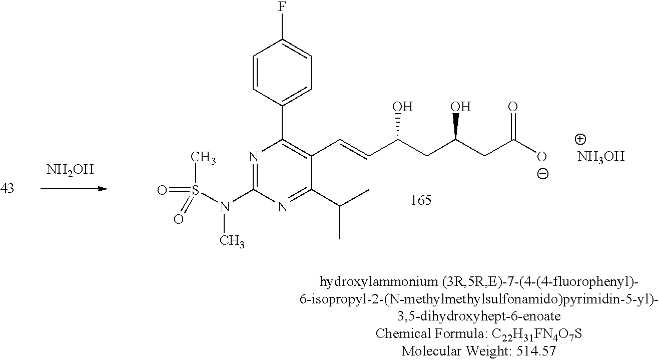

### EXAMPLE 131

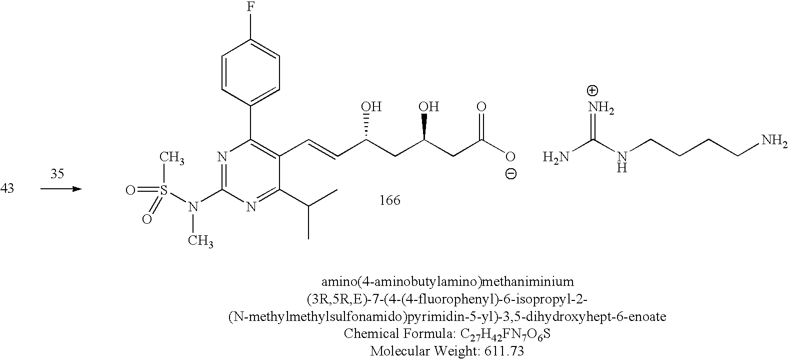

### EXAMPLE 132

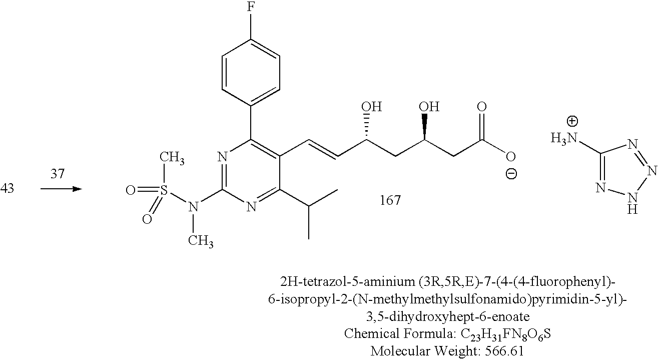

### EXAMPLE 133

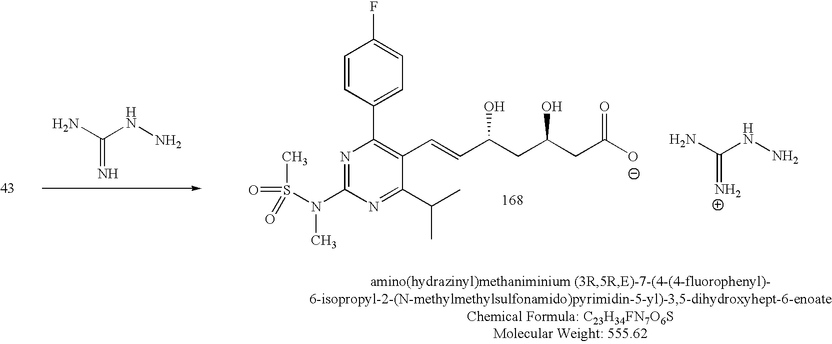

### EXAMPLE 134

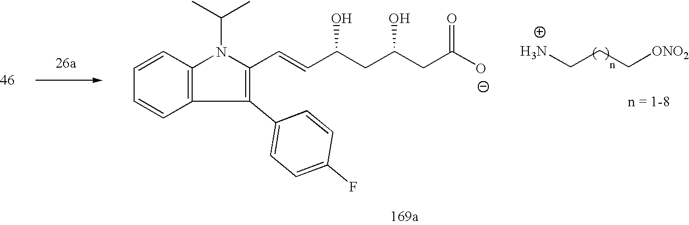

### EXAMPLE 135

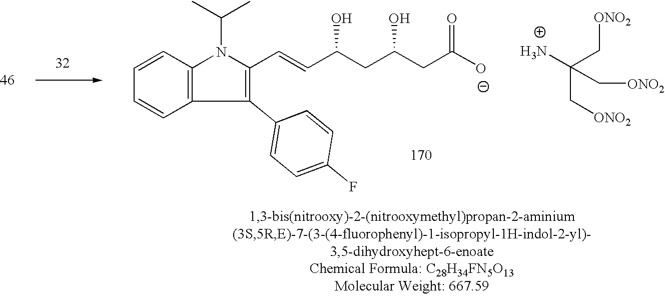

### EXAMPLE 136

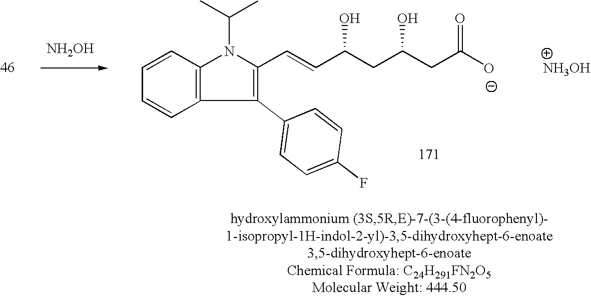

### EXAMPLE 137

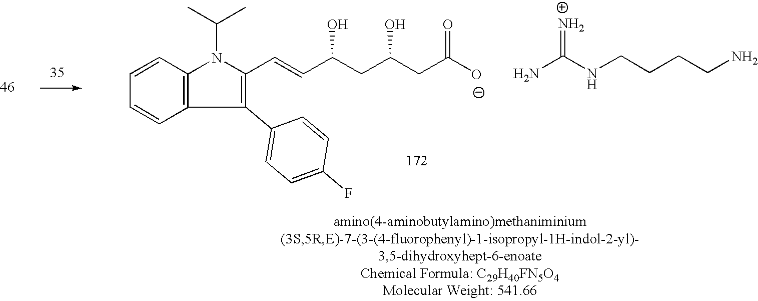

### EXAMPLE 138

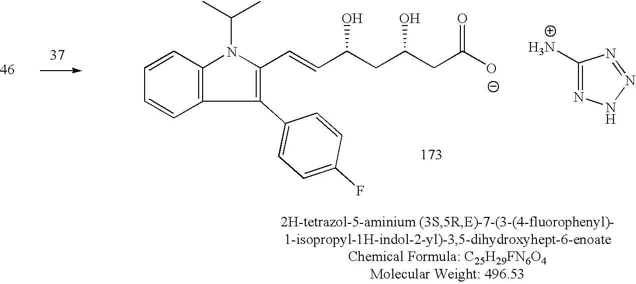

### EXAMPLE 139

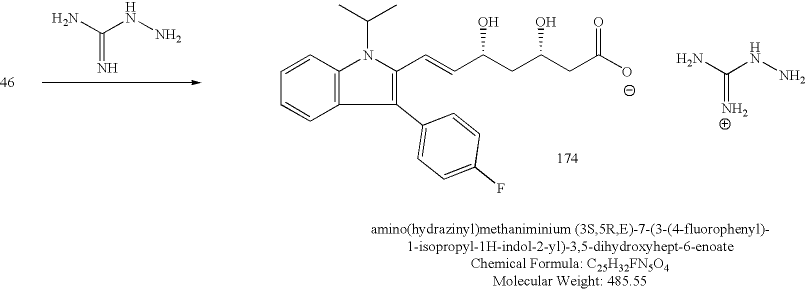

Although the foregoing Examples demonstrate and depict synthesis routes of particular embodiments of fibric acid and statin derivative compounds described herein, this has been done for purposes of illustration only, and is not intended to be limiting to a particular fibric acid or statin compound, to the way in which the fibric acid or statin compound is linked to another molecule, or to be limiting with respect to the scope of the appended claims that follow. Using the knowledge contained within the present disclosure and using the manner of making the various embodiments of fibric acid and statin derivative compounds illustrated herein, those skilled in the art would recognize that other fibric acid and/or statin derivative compounds could be readily formulated by using molecules and linkages other than those illustrated and described herein. Such other fibric acid and/or statin derivative compounds could be achieved by routine experimentation and would function in a similar manner to accomplish the same objectives as those fibric acid and statin derivative compounds illustrated and described herein, and thus, should not be considered as departing from the true scope and spirit of the present disclosure.

### Liver Function Test

Compounds of the present disclosure can be assayed for activity in liver function tests (Kaplan MM. Laboratory tests. In: Schiff L, Schiff E R, eds. Diseases of the liver. 7th ed. Philadelphia: Lippincott, 1993: 108-44). Liver function tests are commonly performed clinical laboratory tests which assay for a number of protein, enzymatic, metabolite and other cellular properties and activities. For example, one enzyme commonly assayed in liver function tests is alanine aminotransferase (ALT; also known as SGPT) which is produced in hepatocytes and is involved in liver degradation. ALT elevation is associated with numerous liver disorders and is a consequence of certain medications which inhibit liver function. A second commonly assayed enzyme in liver function tests is aspartate Aminotransferase (AST; also known as SGOT). AST is needed for energy utilization and is present in muscle, liver and other organs. AST elevation has been associated with liver disease and other conditions (for example, early in the course of a heart attack).

### LXR Antagonist Activity

The ability of compounds of the present disclosure to antagonize liver X receptor (LXR) activity can be determined using assays similar to those described in Thomas et al. (2003) J Biol Chem 278:2403-10. Briefly, the protocol is as follows. The human hepatocellular carcinoma cell line, HepG2 (HB-8065; American Type Culture Collection, Manassas, Va.), is maintained in monolayer culture at 37° C. in 5% CO₂. For transient transfection of HEK293 cells, 4×10⁴ cells are plated into 24-well dishes. HEK293 cells are co-transfected either with either 100 ng of pGL3B-E1b-3XLXRE (LXR response element luciferase reporter plasmid), 100 ng of pCMV6 LXRα (NR1H3, accession number NM_{—}005693), and 50 ng of CMV β-galactosidase or with the Gal4-responsive luciferase plasmid (pG5luc; Promega, Madison, Wis.), an expression plasmid encoding LXRα-LBD (amino acids 162-447) fused to the GAL4 DNA-binding domain (pM LXRα-LBD), and CMV β-galactosidase, as control. After transfection, cells are treated either with vehicle only or one or more compounds of the present disclosure. Following 24 hours of treatment, cells are lysed and firefly luciferase activity is measured using standard luciferase substrate reagents (BD PharMingen, San Diego, Calif.) and is corrected using β-galactosidase activity as a transfection control.

### In Vivo Animal Models

The fibrate and statin derivatives described herein, alone and in combination with other therapeutic agents can be tested for effects on lipid and cholesterol profiles in various lipid and cholesterol related animal models.

### Watanabe Rabbit Model

Fibrate and statin derivatives, alone and in combination, are tested for the ability to prevent or arrest atherosclerosis in a rabbit model of familial hypercholesterolemia. Fibrate and statin derivatives can be administered before or after onset of atherosclerosis for example as described in Havel et al. (1989) Arteriosclerosis 9:133-8, Aliev and Burnstock (1998) Histol Histopathol 13:797-817, Fan et al. (1999) Pathol Int. 49:583-94 and Brousseau and Hoeg (1999) J Lipid Res. 40:365-75.

### Rat High Fat, High Cholesterol Diet Model

Fibrate and statin derivatives, alone and in combination, are tested for effects on cholesterol and lipid profiles in a rat high fat diet model for example as described in Ghibaudi et al. (2002) Obes Res 10:956-63, Ricci and Levin (2003) Am J Physiol Regul Integr Comp Physiol 285:R610-8, and Gao et al. (2002) 936:87-90. Alternatively, animals are fed either a normal diet or a chow supplemented with added fat and/or cholesterol as described in Krause et al (1994) Pharm Res Vol 29, No. 4, Nishina (1993) J of Lipid Res vol 43. and Gajda et al. (2007) Animal Lab News. Based on the specific chows used in these reports, we fed animals a diet consisting of 40% fat, 1% cholesterol, and 0.5% cholic acid (catalog #D01061201, Research Diets, New Brunswick, N.J. 08901). After at least a week on this diet, animals (6-8/group) are dosed once daily perorally with 300 uL test compounds in PEG 400 or suitable vehicle. Groups are dosed for at least one week before whole blood samples are taken for analysis. Blood serum is analyzed using Vertical Auto Profile (VAP; Atherotech, Inc., Birmingham, Ala.) analysis similar to as described in Kulkami (2006) Clin Lab Med 26:787-802.

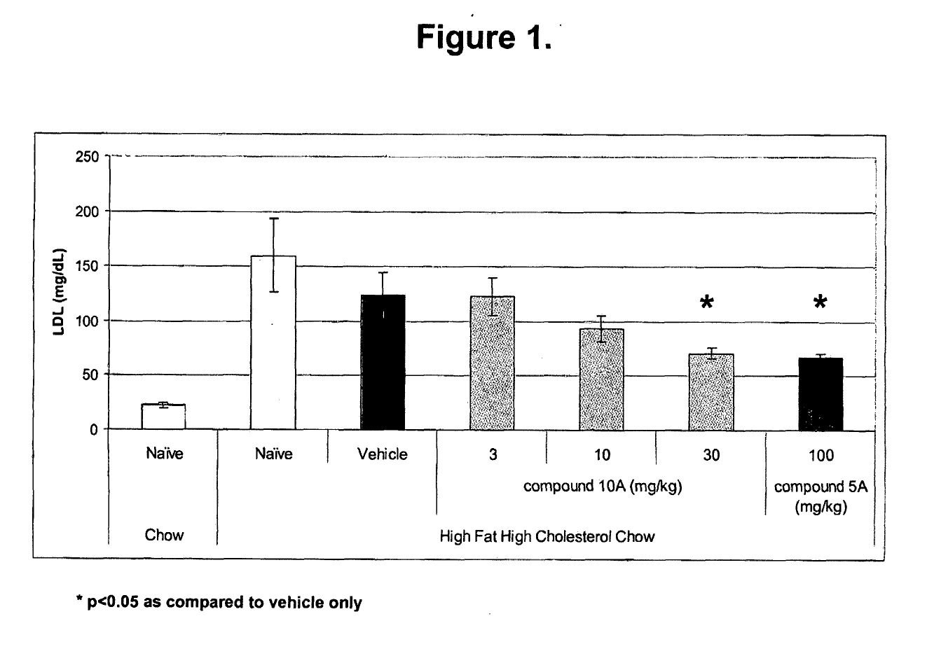

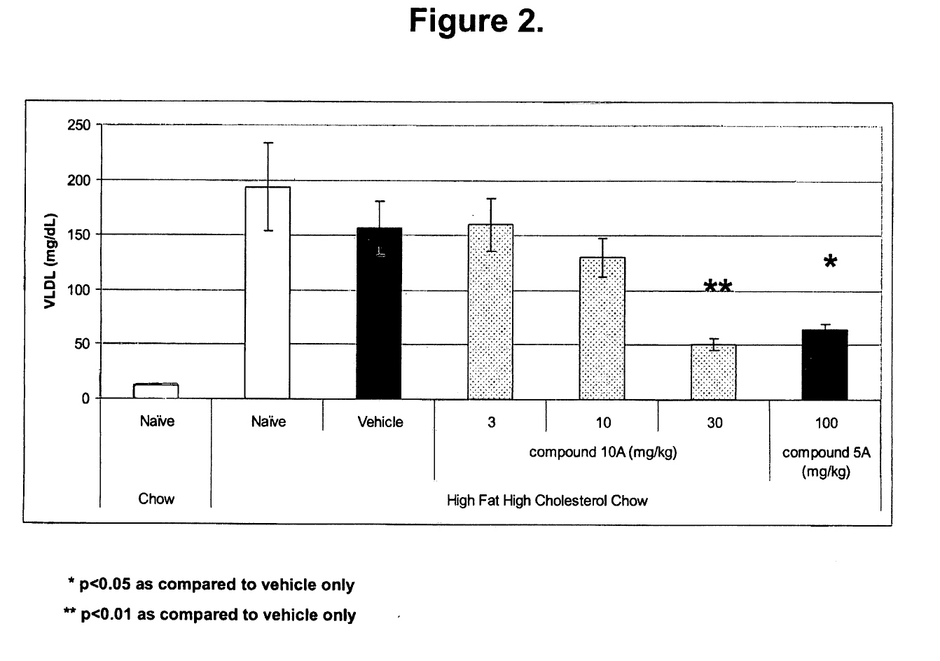

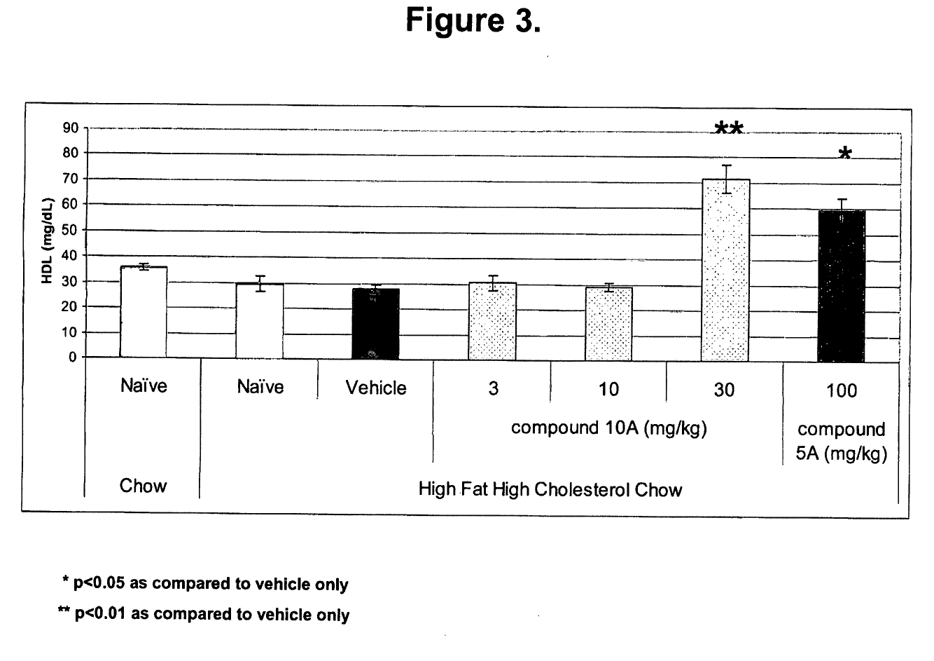

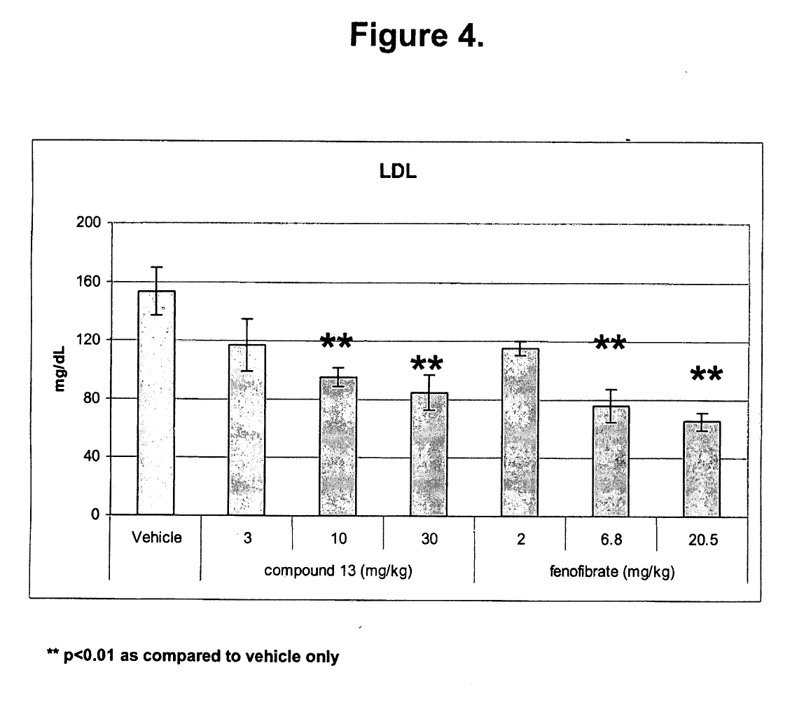

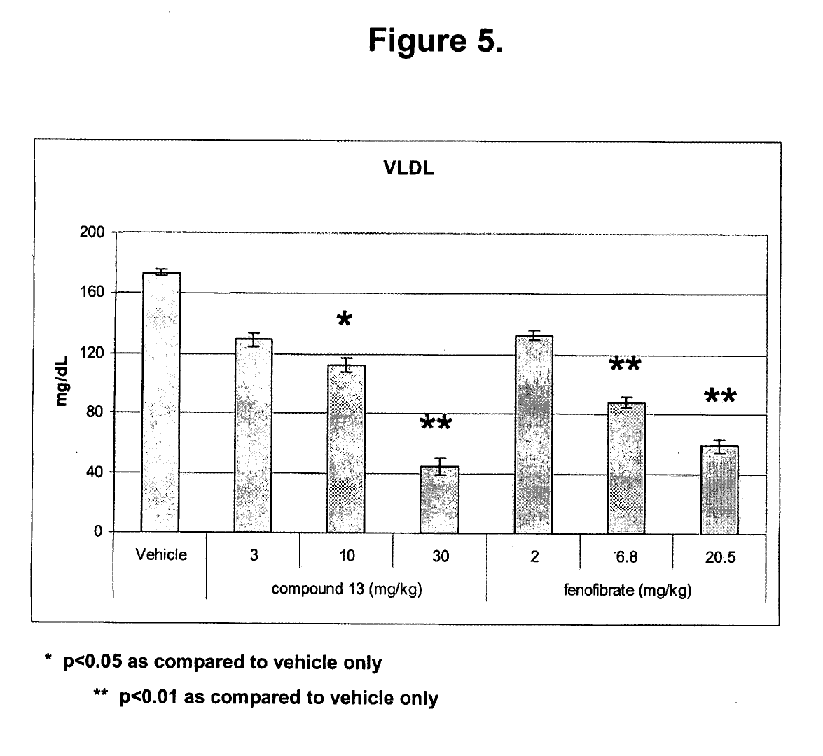

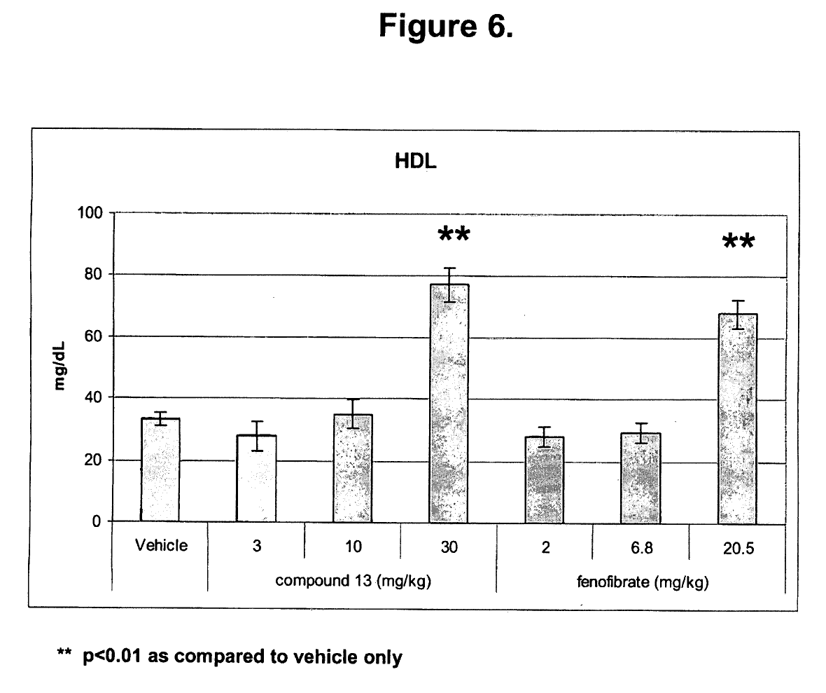

In this assay, Compound 10A [(E)-4-(3,5-dihydroxystyryl)phenyl 2-(4-(4-chlorobenzoyl)phenoxy)-2-methylpropanoate] was associated with dose related decreases of LDL and VLDL, decreased blood glucose, and increased HDL. Similarly, when tested in this assay, Compound 5A [(E)-4-(3,5-dimethoxystyryl)phenyl 2-(4-(4-chlorobenzoyl)phenoxy)-2-methylpropanoate] was associated with decreased LDL and VLDL, no change in blood glucose, and increased HDL. Graphical depictions showing the LDL, VLDL and HDL analyses for compounds 10A and 5A are shown in FIGS. 1, 2, and 3 respectively. Compound 13 (4-(nitrooxy)butyl 2-(4-(4-chlorobenzoyl)phenoxy)-2-methylpropanoate) was also tested in this assay. FIGS. 4-6 are graphs showing that Compound 13 decreased LDL (FIG. 4) and VLDL (FIG. 5) and increased HDL (FIG. 6). Fenofibrate was also tested in this assay at 2, 6.8 and 20.5 mg/kg (FIGS. 4-6) which are equimolar equivalents (for the fenofibric acid moiety) of Compound 10A dosed at 3, and 30 mg/kg. Similar untested dose equivalent amounts to Compound 13 at 3, 10, and 30 mg/kg would be 2.5, 8.3, and 24.8 mg/kg of fenofibrate. In these experiments animals (n=6 per group) were fed either a normal chow (chow) or high fat high cholesterol chow (HFHC; 40% fat, 1% cholesterol, 0.5% cholic acid) diet for one week before test compound administration. Animals received either no test compound (“naïve”), vehicle only (PEG 400) or test compound (compound 10A at 3, 10, or 30 mg/kg, compound 5A at 100 mg/kg, compound 13 at 3, 10, 30 mg/kg, or fenofibrate at 2, 6.8, or 20.5 mg/kg) for seven days before whole blood samples were taken for analysis. Although compound 8A was also tested in this model and actually releases more fenofibric acid than compound 10A, compound 8A does not show any efficacy in this rat high fat, high cholesterol diet model. Compound 8A may be useful for another contemplated use described herein however.

### Obese Mouse Model

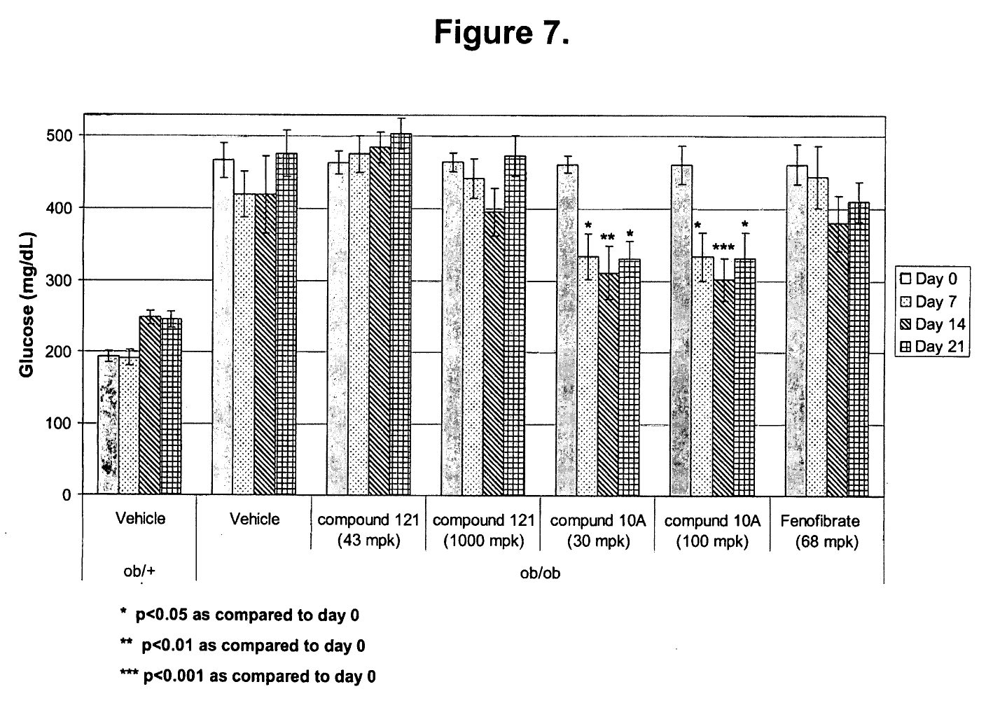

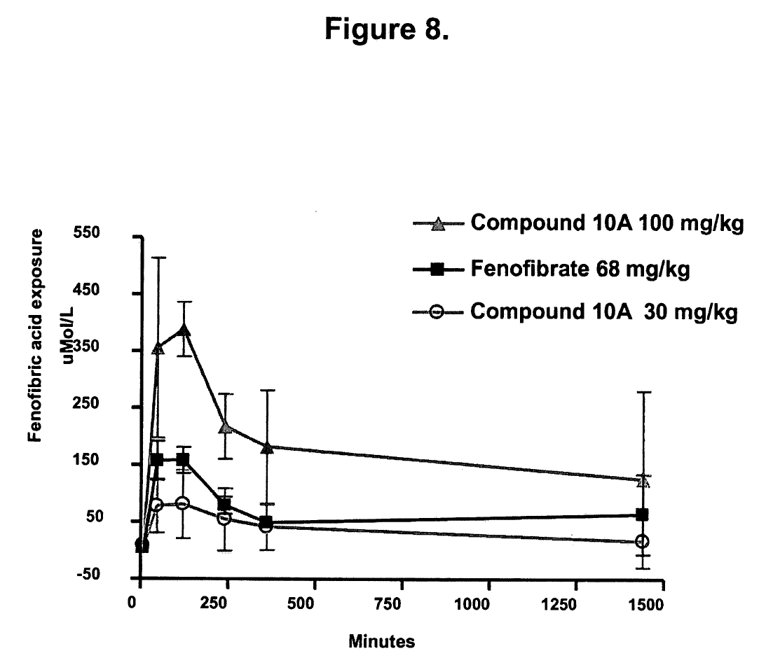

Mice homozygous for the obese (ob) mutation exhibit a diabetes-like syndrome of hyperglycemia, hypercholesterolemia and increased triglycerides (Nishina 1994, Metabolism, Vol 43, No 5). The increased expression of plasma lipids in ob/ob mice make them a useful model for the study of drugs designed to lower cholesterol. In these experiments animals (n=10 per group) were fed a high fat chow (HF; 60% fat,) for one week before test compound administration and continued on this diet for the duration of the experiment. Animals received either vehicle only (PEG 400), or test compound (Compound 121 (resveratrol) at 43 or 1000 mg/kg, Compound 10A at 30 or 100 mg/kg, or fenofibrate at 68 mg/kg) perorally once daily for a total of three weeks. Blood samples were collected weekly for the determination of fasting glucose levels (One touch Ultra, LifeScan Inc). Blood serum was analyzed after three weeks using Vertical Auto Profile (VAP; Atherotech, Inc., Birmingham, Ala.) analysis similar to as described in Kulkami (2006) Clin Lab Med 26:787-802. Compound 10A exhibited statistically significant (when compared to day 0) decreases in blood glucose at days 7, 14, and 21 when tested at both 30 and 100 mg/kg (FIG. 7). In contrast, neither Compound 121 (resveratrol; dosed at 43 or 1000 mg/kg) or fenofibrate (dosed at 68 mg/kg) resulted in statistically significant changes in blood glucose at any time point examined (FIG. 7). 43 mg/kg of compound 121 (resveratrol) and 68 mg/kg of fenofibrate are the equimolar equivalents to the amounts of resveratrol and fenofibric acid moieties in 100 mg/kg of Compound 10A. Although fenofibrate (100 mg/kg) has been shown to decrease blood glucose in ob/ob mice (McCarmona et al. (2005) Intl J. Obesity 29:864-871), fenofibric acid exposure alone does not fully explain the present observation that both 30 and 100 mg/kg of compound 10A decrease blood glucose while 68 mg/kg of fenofibrate does not. FIG. 8 is a pharmacokinetic profile (using the methodologies as described herein) analyzing fenofibric acid levels in blood plasma of ob/ob mice dosed with either 68 mg/kg of fenofibrate, or 30 mg/kg or 100 mg/kg of Compound 10A. There is less fenofibric acid exposure upon dosing with 30 mg/kg Compound 10A as compared to 68 mg/kg fenofibrate (FIG. 8), however the former and not the latter decreases blood glucose (FIG. 7). This data demonstrates the synergistic effect in function between resveratrol and fenofibrate that neither compound exhibits alone, but only in the context of Compound 10A.

### Hamster High Fat Diet Model

Fibrate and statin derivatives, alone and in combination, are tested for effects on cholesterol and lipid profiles in a hamster high fat diet model for example as described in Wang et al. (2001) Eur J. Pharmacol 427:285-93 and van Heek et al. (2001) Diabetes 50:1330-5.

### Measurement of Pharmacokinetic Parameters

Various pharmacokinetic parameters were calculated by collecting plasma samples from animals dosed with a test compound and analyzed by LC/MS-MS. Blood samples were collected at specified time points into tubes containing NaEDTA/NaF. Additional amounts of NaF and NaEDTA were added to the tubes prior to collection so the final concentration was 4 mg/mL of each NaF and NaEDTA. All blood samples were placed on wet ice (or an ice block) and protected from light exposure following collection. The samples were centrifuged and the plasma was separated and immediately transferred into amber tubes upon centrifugal separation. Following separation, 40 μL of 5 M sodium formate pH 4.0 buffer was added for every 1 mL of plasma collected. Samples were stored frozen within 15 minutes of centrifugation at approximately −80° C. Samples (10 μL injections) were separated using a reverse phase HPLC column (Thermo Electron Hypersil Gold C18, 2.1×50 mm, 5 um particle size) with gradient elution (Mobile Phase A:0.05% v/v acetic acid in 95:5 water:acetonitrile; Mobile Phase B: 0.05% acetic acid v/v in 5:95 water:acetonitrile) at 0.4 mL/min with a gradient-time profile as follows: initial condition of 15% B, ramping to 40% B over 1.5 min, ramping to 55% B over the next 0.5 min, ramping to 100% B over the next 0.5 minutes with a 1.6 min hold at 100% B. The column was allowed to re-equilibrate at 15% B for 0.9 min. A Waters Quattro micro (Waters Corp.; Milford, Mass.) triple quadrupole mass spectrometer operating in MRM mode was used to detect test compounds as they eluted from the HPLC column using characteristic precursor-to-product ion transitions. Concentrations were determined by relative responses to an internal standard and calculated based on a standard concentration curve of the test compound. MassLynx software (Waters, Corp.; Milford, Mass.) was used to calculate the absolute concentration of the test compound in each plasma sample and exported into Microsoft Excel (Microsoft Corp., Redmond, Wash.) or Graphpad Prism (GraphPad Software, Inc., San Diego, Calif.) for analysis. A concentration versus time plot was generated from the data in Graph Pad Prism to generate PK curves and calculate the AUCₙ (Area Under the Curve, n=length of experiment in hours) for both intravenous (IV) and orally dosed animals. Oral Bioavailability (Fₙ) was calculated using the equation: F=(AUCₒᵣₐₗ/AUC_{IV})*Dose_{IV}.Doseₒᵣₐₗ). Cₘₐₓ and Tₘₐₓ are determined by visual inspection of the oral concentration curve. Cₘₐₓ is the maximum concentration of the test compound circulating in the blood through the duration of the experiment reported at time, T (Tₘₐₓ).

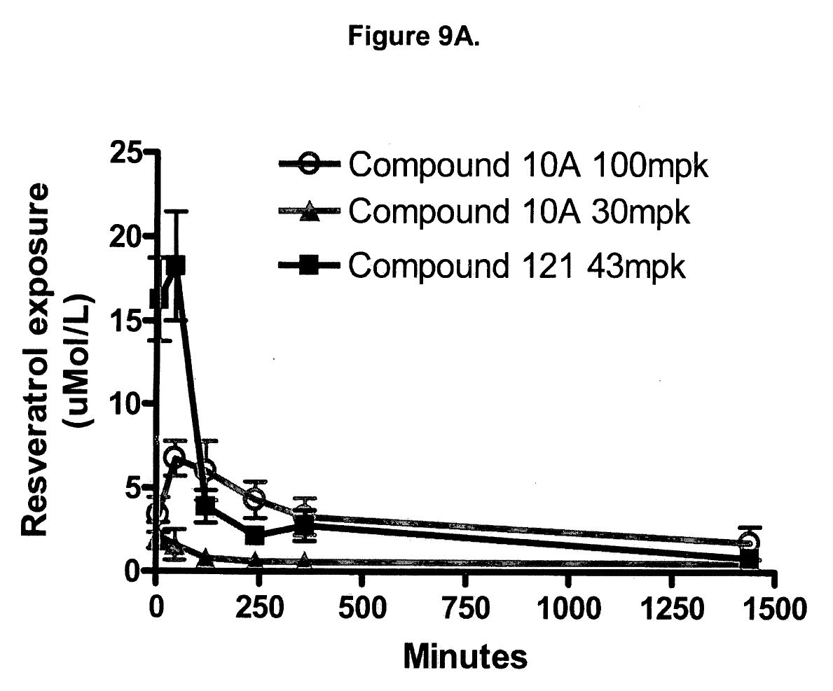

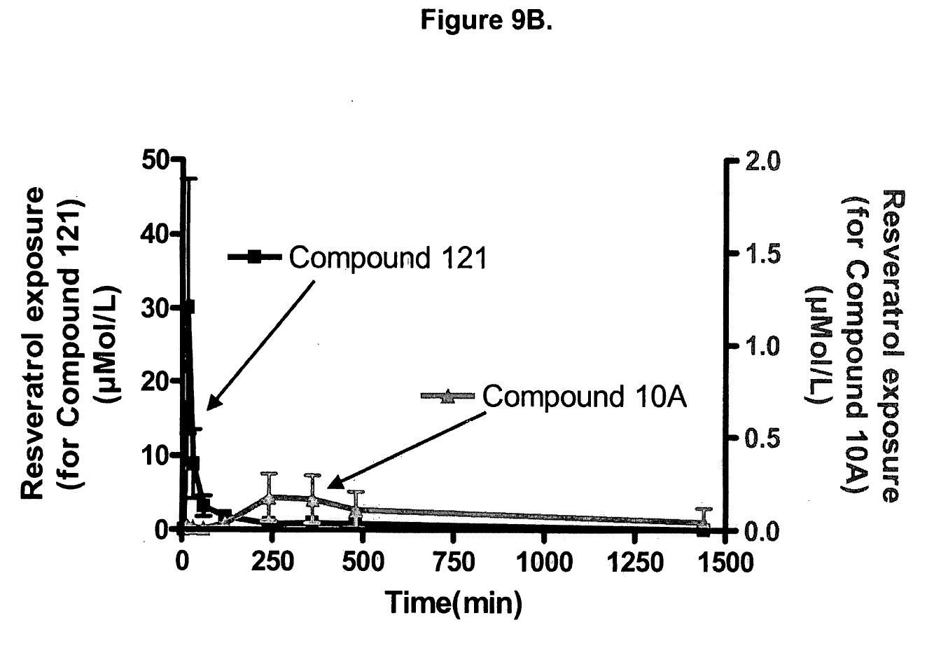

Resveratrol pharmacokinetic profiles were determined after oral administration of either Compound 10A or Compound 121 (resveratrol) in either the obese mouse model (FIG. 9A) or rat high fat high cholesterol diet model (FIG. 9B) as described herein. In both rodent species, Compound 10A generated a different resveratrol pharmacokinetic profile compared to administration of resveratrol (compound 121) alone, evidencing that Compound 10A possesses some added function that neither fenofibrate nor resveratrol exhibits alone. In FIG. 9A, 43 mg/kg of compound 121 (resveratrol) is the equimolar equivalent to the amount resveratrol moiety in 100 mg/kg of Compound 10A.

Combination Therapy with PDE Inhibitors

The fibric acid and statin derivative compounds and compositions described herein can be used in combination therapy (for example in methods to treat a lipid related disorder) with one or more phosphodiesterase inhibitors. Phosphodiesterase (PDE) inhibitors slow the degradation of cyclic AMP (cAMP) and/or cyclic GMP (cGMP) by inhibition of the phosphodiesterases, which can lead to a relative increase in the intracellular concentration of cAMP and cGMP. Thus, co-administration of fibric acid and statin derivative compounds or compositions and PDE inhibitors which slow the degradation of cGMP by phosphodiesterases is useful for treating lipid metabolism related disorders.

PDE inhibitors include PDE3 inhibitors, PDE4 inhibitors, PDE5 inhibitors, and inhibitors with multiple specificity including PDE3/4 and PDE3/4/5 inhibitors. Specific PDE inhibitors include those disclosed in patent publications DE1470341, DE2108438, DE2123328, DE2305339, DE2305575, DE2315801, DE2402908, DE2413935, DE2451417, DE2459090, DE2646469, DE2727481, DE2825048, DE2837161, DE2845220, DE2847621, DE2934747, DE3021792, DE3038166, DE3044568, EP000718, EP0008408, EP0010759, EP0059948, EP0075436, EP0096517, EP0112987, EP0116948, EP0150937, EP0158380, EP0161632, EP0161918, EP0167121, EP0199127, EP0220044, EP0247725, EP0258191, EP0272910, EP0272914, EP0294647, EP0300726, EP0335386, EP0357788, EP0389282, EP0406958, EP0426180, EP0428302, EP0435811, EP0470805, EP0482208, EP0490823, EP0506194, EP0511865, EP0527117, EP0626939, EP0664289, EP0671389, EP0685474, EP0685475, EP0685479, JP92234389, JP94329652, JP95010875, U.S. Pat. No. 4,963,561, U.S. Pat. No. 5,141,931, WO9117991, WO9200968, WO9212961, WO9307146, WO9315044, WO9315045, WO9318024, WO9319068, WO9319720, WO9319747, WO9319749, WO9319751, WO9325517, WO9402465, WO9406423, WO9412461, WO9420455, WO9422852, WO9425437, WO9427947, WO9500516, WO9501980, WO9503794, WO9504045, WO9504046, WO9505386, WO9508534, WO9509623, WO9509624, WO9509627, WO9509836, WO9514667, WO9514680, WO9514681, WO9517392, WO9517399, WO9519362, WO9522520, WO9524381, WO9527692, WO9528926, WO9535281, WO9535282, WO9600218, WO9601825, WO9602541, WO9611917, DE3142982, DE1116676, DE2162096, EP0293063, EP0463756, EP0482208, EP0579496, EP0667345 U.S. Pat. No. 6,331,543, US20050004222 (including those disclosed in formulas I-XIII and paragraphs 37-39, 85-0545 and 557-577), WO9307124, EP0163965, EP0393500, EP0510562, EP0553174, WO9501338 and WO9603399, as well as PDE5 inhibitors (such as RX-RA-69, SCH-51866, KT-734, vesnarinone, zaprinast, SKF-96231, ER-21355, BF/GP-385, NM-702, vardenafil (LEVITRA®); and tadalafil (CIALIS® and sildenafil (Viagra™)), PDE4 inhibitors (such as etazolate, ICI63197, RP73401, imazolidinone (RO-20-1724), MEM 1414 (R1533/R1500; Pharmacia Roche), denbufylline, rolipram, oxagrelate, nitraquazone, Y-590, DH-6471, SKF-94120, motapizone, lixazinone, indolidan, olprinone, atizoram, KS-506-G, dipamfylline, BMY-43351, atizoram, arofylline, filaminast, PDB-093, UCB-29646, CDP-840, SKF-107806, piclamilast, RS-17597, RS-25344-000, SB-207499, TIBENELAST, SB-210667, SB-211572, SB-211600, SB-212066, SB-212179, GW-3600, CDP-840, mopidamol, anagrelide, ibudilast, aminone, pimobendan, cilostazol, quazinone and N-(3,5-dichloropyrid-4-yl)-3-cyclopropylmethoxy-4-difluoromethoxybenzamide, PDE3 inhibitors (such as ICI153, 100, bemorandane (RWJ 22867), MC1-154, UD-CG 212, sulmazole, ampizone, cilostamide, carbazeran, piroximone, imazodan, CI-930, siguazodan, adibendan, saterinone, SKF-95654, SDZ-MKS-492, 349-U-85, emoradan, EMD-53998, EMD-57033, NSP-306, NSP-307, revizinone, NM-702, WIN-62582 and WIN-63291, enoximone and milrinone, PDE3/4 inhibitors (such as benafentrine, trequinsin, ORG-30029, zardaverine, L-686398, SDZ-ISQ-844, ORG-20241, EMD-54622, and tolafentrine) and other PDE inhibitors (such as vinpocetin, papaverine, enprofylline, cilomilast, fenoximone, pentoxifylline, roflumilast, and theophylline.

The present disclosure provides, in various embodiments, pharmaceutical combination kits and oral drug dosage forms that contain at least one fibric acid and/or statin derivative composition and at least one lipid altering agent or phosophodiesterase inhibitor. In other embodiments, the present disclosure provides pharmaceutical combinations kits and oral dosage forms that contain at least one fibric acid and/or statin derivative composition and at least one lipid altering agent and/or at least one phosophodiesterase inhibitor. The therapeutic agents may be contained in the same oral dosage form or in separate dosage forms that are administered sequentially. When more than two therapeutic agents are present in the pharmaceutical combination kit, all agents may be present in the same or different dosage forms and may be administered sequentially or simultaneously.

The active ingredients used in oral formualtions, i.e., fibric acid and statin derivative compositions, either alone or in combination with one or more additional agents (e.g., lipid altering agents or PDE inhibitors), are well known in the art and many are commercially available. If desired, drugs can also be manufactured using methodology well known in the art.

### Formulation and Administration

The pharmaceutical compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Compositions of the present disclosure may also optionally include other therapeutic ingredients, anti-caking agents, preservatives, sweetening agents, colorants, flavors, desiccants, plasticizers, dyes, and the like. Any such optional ingredient must, of course, be compatible with the compound of the disclosure to insure the stability of the formulation.

Examples of excipients for use as the pharmaceutically acceptable carriers and the pharmaceutically acceptable inert carriers and the aforementioned additional ingredients include, but are not limited to:

Binders: corn starch, potato starch, other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch (e.g., STARCH 1500® and STARCH 1500 LM®, sold by Colorcon, Ltd.), hydroxypropyl methyl cellulose, microcrystalline cellulose (e.g. AVICEL™, such as, AVICEL-PH-101™, -103™ and -105™, sold by FMC Corporation, Marcus Hook, Pa., USA), or mixtures thereof;

Fillers: talc, calcium carbonate (e.g., granules or powder), dibasic calcium phosphate, tribasic calcium phosphate, calcium sulfate (e.g., granules or powder), microcrystalline cellulose, powdered cellulose, dextrates, kaolin, mannitol, silicic acid, sorbitol, starch, pre-gelatinized starch, or mixtures thereof,

Disintegrants: agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, clays, other algins, other celluloses, gums, or mixtures thereof,

Lubricants: calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laurate, agar, syloid silica gel (AEROSIL 200, W.R. Grace Co., Baltimore, Md. USA), a coagulated aerosol of synthetic silica (Degussa Co., Plano, Tex. USA), a pyrogenic silicon dioxide (CAB-O-SIL, Cabot Co., Boston, Mass. USA), or mixtures thereof,

Anti-caking agents: calcium silicate, magnesium silicate, silicon dioxide, colloidal silicon dioxide, talc, or mixtures thereof,

Antimicrobial agents: benzalkonium chloride, benzethonium chloride, benzoic acid, benzyl alcohol, butyl paraben, cetylpyridinium chloride, cresol, chlorobutanol, dehydroacetic acid, ethylparaben, methylparaben, phenol, phenylethyl alcohol, phenylmercuric acetate, phenylmercuric nitrate, potassium sorbate, propylparaben, sodium benzoate, sodium dehydroacetate, sodium propionate, sorbic acid, thimersol, thymo, or mixtures thereof, and

Coating agents: sodium carboxymethyl cellulose, cellulose acetate phthalate, ethylcellulose, gelatin, pharmaceutical glaze, hydroxypropyl cellulose, hydroxypropyl methylcellulose, hydroxypropyl methyl cellulose phthalate, methylcellulose, polyethylene glycol, polyvinyl acetate phthalate, shellac, sucrose, titanium dioxide, carnuba wax, microcrystalline wax, or mixtures thereof.

It can be useful to administer a fibric acid or statin derivative composition or pharmaceutical formulation described herein together with an HMG-CoA reductase inhibitor such as a statin, or with a PDE inhibitor. It can be particularly useful to combine a fibric acid or statin derivative composition described herein together and an HMG-CoA reductase inhibitor such as a statin, or a PDE inhibitor, in a single pharmaceutical composition. The precise amount of each of these two active ingredients in a dosage unit will depend on the desired dosage of each component. Thus, it can be useful to create a dosage unit that will, when administered according to a particular dosage schedule (e.g., a dosage schedule specifying a certain number of units and a particular timing for administration), deliver the same dosage of each component as would be administered if the patient was being treated with only a single component. In other circumstances, it might be desirable to create a dosage unit that will deliver a dosage of one or both components that is less than that which would be administered if the patient was being treated only with a single component. Finally, it might be desirable to create a dosage unit that will deliver a dosage of one or both components that is greater than that which would be administered if the patient was being treated only with a single component. The pharmaceutical composition can include additional ingredients such as stabilizers or bulking agents.

Making of Pharmaceutical Preparations: The active agents used in the compositions of the present disclosure will typically be formulated in accordance with methods that are standard in the art (see e.g., Remington's Pharmaceutical Sciences, 16th edition, A. Oslow, editor, Easton, Pa. (1980)). Drugs may be prepared in admixture with conventional excipients, carriers, buffers, flavoring agents, etc. Typical carriers include, but are not limited to: water; salt solutions; alcohols; gum arabic; vegetable oils; benzyl alcohols; polyethylene glycols; gelatin; carbohydrates, such as lactose, amylose or starch; magnesium stearate; talc; silicic acid; paraffin; perfume oil; fatty acid esters; hydroxymethylcellulose; polyvinyl pyrrolidone; etc. Pharmaceutical preparations can be sterilized and, if desired, mixed with auxiliary agents such as: lubricants; preservatives; disintegrants; stabilizers such as cyclodextrans; wetting agents; emulsifiers; salts; buffers; natural or artificial coloring agents; natural or artificial flavoring agents; or aromatic substances. Pharmaceutical preparations can also include one or more of the following: acetylated monoglyceride, aspartame, beta carotene, calcium stearate, carnauba wax, cellulose acetate phthalate, citric acid, citric acid anhydrous, colloidal silicon dioxide, confectioner's sugar, crospovidone, docusate sodium, ethyl alcohol, ferric oxide, fructose, gelatin, glycerine, glyceryl monostearate (e.g. glyceryl monostearate 40-50), glyceryl triacetate, HPMC (hydroxypropyl methylcellulose), hydroxypropyl cellulose, hypromellose, iron oxide, isopropyl alcohol, lactose monohydrate, low substituted hydroxypropyl cellulose, magnesium carbonate, magnesium stearate, maltol, mannitol, methacrylic acid, methacrylic acid copolymer (e.g. methacrylic acid copolymer type C), methylcellulose, microcrystalline cellulose, mono ammonium glycyrrhizinate, n-butyl alcohol, paraffin, pectin propylene glycol alginate, polyacrylate, polyethylene glycol (e.g. polyethylene glycol 6000), polysorbate 80, polyvinyl pyrrolidone, povidone, propylene glycol, shellac, silicon dioxide, sodium carbonate, sodium citrate, sodium hydroxide, sodium lauryl sulfate, sodium stearyl fumarate, sorbitol, starch, sucrose, sugar sphere, talc, titanium dioxide, triethyl citrate, and xanthan gum. In certain embodiments, buffers that can raise the pH of the stomach are used. For example bicarbonate buffers may be included in the outer coating or as a rapidly dissolving, separate layer immediately below the outer coating.

The enteric coating surrounding the core may be applied using standard coating techniques. Materials used to form the enteric coating may be dissolved or dispersed in organic or aqueous solvents and may include one or more of the following: methacrylic acid copolymers; shellac; hydroxypropylmethylcellulose phthalate; polyvinyl acetate phthalate; hydroxypropylmethylcellulose trimellitate; carboxymethylcellulose; cellulose acetate phthalate; or other suitable enteric coating polymers. The pH at which the enteric coat will dissolve can be controlled by the polymer or combination of polymers selected and/or ratio of pendant groups. For example, dissolution characteristics of the coating can be altered by the ratio of free carboxyl groups to ester groups. Enteric coating layers may also contain pharmaceutical plasticizers such as: triethyl citrate; dibutyl phthalate; triacetin; polyethylene glycols; polysorbates; etc. Additives such as dispersants, colorants, anti-adhering and anti-foaming agents may also be included.

Making of Tablet Dosage Forms: Tablets can be made using standard technology well known in the art. Drugs used in the core or the outer coating may be granulated by methods such as slugging, low-shear or high-shear granulation, wet granulation, or fluidized bed granulation. Outer coatings may be formed by preparing a mixture containing appropriate polymers and a sufficient amount of drug to produce a therapeutically effective dose. The solution may then be sprayed on preformed, enterically-coated cores to produce the final tablets. If desired, a buffer layer or layer containing other agents may be interspersed between the enterically coated core and the outer coating.

In certain embodiments a pharmaceutical composition is prepared by adding a pharmaceutically acceptable carrier to the aforementioned compound, a pharmaceutically acceptable salt thereof, or a hydrate thereof as an active ingredient of the medicament of the present disclosure. As the medicament of the present disclosure, a substance, per se, that is selected from the group consisting of the alkylenedioxybenzene derivative and a pharmaceutically acceptable salt thereof, and a hydrate thereof and a solvate thereof may be administered to a mammal including human. In certain embodiments, pharmaceutical compositions comprising one or more of the aforementioned substances as an active ingredient and one or more of pharmaceutical additives are administered to a patient.

A variety of administration routes can be used in accordance with the present disclosure. An effective amount of the composition described herein can be administered parenterally, orally, by inhalation, nasally, buccally, or via an implanted reservoir. In certain embodiments the composition is administered orally. In certain embodiments oral sustained/extended release formulations are used.

Examples of the pharmaceutical composition include formulations for oral administration such as tablets, capsules, subtilized granules, powders, pills, troches, sublingual tablets and liquid preparations, and formulations for parenteral administration such as injections, suppositories, ointments, patches and the like.

In certain embodiments, formulations including those which slowly release the agent over time (i.e., sustained/extended release), such as found in lozenges, gums, and buccal patches are used. In other embodiments, formulations including agents in a bioadherent ingestible composition, such as those found in U.S. Pat. Nos. 5,858,391 and 5,670,163 to Cuca, et al. are used. The agent may also be formulated as a liquid or as a tablet, pill, capsule or powder to be dissolved in a liquid, and is preferably slowly sipped by the patient.

Tablets and capsules for oral administration are usually provided in a unit dosage form, and can be prepared by adding ordinary pharmaceutical carriers such as binders, fillers, diluents, compressing agents, lubricants, disintegrating agents, coloring matters, flavoring agents, and moistening agents. Tablets may be coated according to a well-known method, for example, by using an enteric coating agent. For example, fillers such as cellulose, mannitol and lactose; disintegrating agents such as starch, polyvinylpyrrolidone, starch derivatives and sodium starchglycolate; lubricants such as magnesium stearate; moistening agents such as sodium laurylsulfate and the like may be used.

Liquid preparations for oral administration can be provided in the forms of, for example, aqueous or oily suspensions, solutions, emulsions, syrups and elixirs, as well as dried formulations that are re-dissolvable before use by water or a suitable medium. Those liquid preparations may contain ordinary additives, for example, suspending agents such as sorbitol, syrups, methylcellulose, gelatin, hydroxyethylcellulose, carboxymethylcellulose, aluminum stearate gel and hydrogenated edible fats; emulsifiers such as lecitin, sorbitan monooleate and gum arabic; non-aqueous media including edible oils such as almond oil, rectified coconut oil, oily esters (e.g., esters of glycerin), propylene glycol and ethyl alcohol; preservatives such as methyl ester, ethyl ester and propyl ester of p-hydroxybenzoic acid and sorbic acid; and usual flavoring agents and coloring matters as required.

Formulations for oral administration can be manufactured according to a method well known in the art, for example, by mixing, filling, compressing and the like. In addition, it is also possible to disperse the active ingredient in a formulation containing a large amount of filler by repetitive mixing. Formulations for parenteral administration are generally provided as unit dosage form preparations containing the compound as the active ingredient and a sterilized medium. The solution for parenteral administration may generally be prepared by dissolving the compound in a medium, subjecting the resulting solution to filtration for sterilization, filling the solution in vials or ampoules, and sealing the vials or ampoules. It is also possible to freeze the composition and fill the result in vials, and then eliminate the moisture in vacuo to improve stability. Parenteral suspensions can be prepared by substantially the same method as that applied to solutions for parenteral administration; however, the suspensions can preferably be manufactured by suspending the active ingredient in a medium, and then subjecting the result to sterilization by using ethylene oxide or the like. Furthermore, surface active agents, moistening agents and so forth may also be added so that a uniform dispersion of the active ingredient can be obtained.

Combining two or more active ingredients in single dosage form results in the possibility of chemical interactions between the active drug substances. For example, acidic and basic active ingredients can react with each other and acidic active ingredients can facilitate the degradation of acid labile substances. Thus, in certain dosage forms, acidic and basic substances can be physically separated as two distinct or isolated layers in a compressed tablet, or in the core and shell of a press-coated tablet. Additional agents that are compatible with acidic as well as basic substances, have the flexibility of being placed in either layer. In certain multiple layer compositions at least one active ingredient can be enteric-coated. In certain embodiments thereof at least one active ingredient can be presented in a controlled release form. In certain embodiments where a combination of three or more active substances are used, they can be presented as physically isolated segments of a compressed mutlilayer tablet, which can be optionally film coated.

The therapeutic combinations described herein can be formulated as a tablet or capsule comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

The therapeutic combinations described herein can be formulated as a capsule comprising microtablets or minitablets of all active ingredients. Microtablets of the individual agents can be prepared using well known pharmaceutical procedures of tablet making like direct compression, dry granulation or wet granulation. Individual microtablets can be filled into hard gelatin capsules. A final dosage form may comprise one or more microtablets of each individual component. The microtablets may be film coated or enteric coated.

The therapeutic combinations described herein can be formulated as a capsule comprising one or more microtablets and powder, or one or more microtablets and granules or beads. In order to avoid interactions between drugs, some active ingredients of a said combination can be formulated as microtablets and the others filled into capsules as a powder, granules, or beads. The microtablets may be film coated or enteric coated. At least one active ingredient can be presented in controlled release form.

The therapeutic combinations described herein can be formulated wherein the active ingredients are distributed in the inner and outer phase of tablets. In an attempt to divide chemically incompatible components of proposed combination, few interacting components are converted in granules or beads using well-known pharmaceutical procedures in prior art. The prepared granules or beads (inner phase) are then mixed with outer phase comprising the remaining active ingredients and at least one pharmaceutically acceptable excipient. The mixture thus comprising inner and outer phase is compressed into tablets or molded into tablets. The granules or beads can be controlled release or immediate release beads or granules, and can further be coated using an enteric polymer in an aqueous or non-aqueous system, using methods and materials that are known in the art.

The therapeutic combinations described herein can be formulated as single dosage unit comprising suitable buffering agent. All powdered ingredients of said combination are mixed and a suitable quantity of one or more buffering agents is added to the blend to minimize possible interactions.

The agents described herein, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques. The agents described herein, alone or in combination, can be formulated using Nanocrystal® technology (Elan Corporation, Dublin, Ireland).

The agents can be a free acid or base, or a pharmacologically acceptable salt thereof. Solids can be dissolved or dispersed immediately prior to administration or earlier. In some circumstances the preparations include a preservative to prevent the growth of microorganisms. The pharmaceutical forms suitable for injection can include sterile aqueous or organic solutions or dispersions which include, e.g., water, an alcohol, an organic solvent, an oil or other solvent or dispersant (e.g., glycerol, propylene glycol, polyethylene glycol, and vegetable oils). The formulations may contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Pharmaceutical agents can be sterilized by filter sterilization or by other suitable means.

Suitable pharmaceutical compositions in accordance with the disclosure will generally include an amount of the active compound(s) with an acceptable pharmaceutical diluent or excipient, such as a sterile aqueous solution, to give a range of final concentrations, depending on the intended use. The techniques of preparation are generally well known in the art, as exemplified by Remington's Pharmaceutical Sciences, 18th Ed., Mack Publishing Company, 1995.

The agent can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylamino ethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include tris, arecoline, arginine, barium, betaine, bismuth, chloroprocaine, choline, clemizole, deanol, imidazole, and morpholine ethanol.

The agents of the disclosure can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein.

### Dosing and Regimen

Doses of the aforementioned compounds as the active ingredient can be suitably decided depending on the purpose of administration, i.e., therapeutic or preventive treatment, nature of a disease to be treated or prevented, conditions, body weight, age, gender and the like of a patient. In the method for administering the pharmaceutical preparation according to the present disclosure, the fibric acid or statin derivative compositions may be administered simultaneously with a lipid altering agent or PDE inhibitor, or the two may be sequentially administered in an optional order. The practically desirable method and sequence for administration varies depending on the purpose of administration, i.e., therapeutic or preventive treatment, nature of a disease to be treated or prevented, conditions, body weight, age, gender and the like of a patient. The optimum method and sequence for administration of the compounds described in detail herein under preset given conditions may be suitably selected by those skilled in the art with the aid of the routine technique and the information contained in the present specification and field of disclosure. In certain embodiments, an amount of about 0.05 mg to 20 mg, about 0.05 mg to 10 mg, about 0.01 mg to 3 mg, about 1 mg to 3 mg, about 0.1 mg to 1 mg, about 0.5 mg to 3 mg, about 2 mg to 3 mg, about 1 mg to 5 mg, or about 2 mg to 5 mg of a fibric acid or statin derivative composition per day for an adult can be orally administered in the formulation of a sublingual tablet, buccal tablet, extended-release (long-acting) capsule, or spray. In certain embodiments, an amount of about 10 mg to 120 mg, about 10 mg to 90 mg, about 30 mg to 60 mg, about 60 mg to 100 mg, or about 20 mg to 60 mg (e.g. 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg) of a fibric acid or statin derivative composition per day for an adult can be orally administered in the formulation of a sublingual tablet, buccal tablet, extended-release (long-acting) capsule, or spray. When administered in combination with a lipid altering agent where the lipid altering agent is a statin, about 2 mg to 80 mg, about 5 mg to 40 mg, or about 10 to 80 mg of a statin per day for an adult can be orally administered. When administered in combination with a lipid altering agent where the lipid altering agent is a bile acid sequestrant, about 1 g to 30 g, about 0.2 g to 6 g, about 0.1 g to 3 g, about 0.02 g to 0.6 g, about 0.01 g to 0.3 g, about 5 g to 150 g, about 2 g to 60 g or about 10 g to 300 g of a bile acid sequestrant (e.g. cholestyramine, colesevelam or colestipol) per day for an adult can be orally administered. When administered in combination with a lipid altering agent where the lipid altering agent is a fibrate about 5 mg to 150 mg of a fibrate (e.g. fenofibrate (Tricor®)) per day for an adult can be orally administered. When administered in combination with a lipid altering agent where the lipid altering agent is a cholesterol absorption inhibitor (e.g. ezetimibe), about 2 mg to 80 mg, about 5 mg to 40 mg, or about 10 to 80 mg of a cholesterol absorption inhibitor per day for an adult can be orally administered. When administered in combination with a lipid altering agent where the lipid altering agent is an sGC modulator (e.g., nitroglycerin), about 0.05 mg to 20 mg, about 0.05 mg to 10 mg, about 0.01 mg to 3 mg, about 1 mg to 3 mg, about 0.1 mg to 1 mg, about 0.5 mg to 3 mg, about 2 mg to 3 mg, about 1 mg to 5 mg, or about 2 mg to 5 mg of an sGC modulator per day for an adult can be orally administered. When administered in combination with a PDE inhibitor, about 1 mg to 100 mg of a PDE inhibitor per day for an adult can be orally administered (e.g., 25 mg, 50 mg, or 100 mg of sildenafil; 2.5 mg, 5 mg, 10 mg, or 20 mg of vardenafil; or 5 mg, 10 mg, or 20 mg of tadalafil). Such doses may be desirably administered once a day to several times a day as divided portions. For example, the compositions of the present disclosure may be administered at least 1×, 2×, 3×, 4×, 5×, 6×, 8×, 10× or 20× a day. In certain embodiments the composition described herein is administered at least once a day for a period of days, weeks, months or years. The agent may be administered at least once, twice, three, or four times daily. Depending upon the desired therapeutic action, patient response and other factors, the dosage form may be administered between meals, during meals, prior to a meal (i.e., within 5, 10, 15, 20, 25, 30, 35, 40, 45, 50, 55, or 60 minutes, 2 hours, 4 hours, 8 hours, or 12 hours prior to eating) or after a meal (i.e., within 5, 10, 15, 20, 25, 30, 35, 40, 45, 50, 55, or 60 minutes, 2 hours, 4, hours, 8 hours, or 12 hours following a meal). In certain embodiments the dosage unit and daily dose are equivalent. In various embodiments, the dosage unit is administered with food at anytime of the day, without food at anytime of the day, with food after an overnight fast (e.g. with breakfast), at bedtime after a low fat snack.

### Kits

The compounds and pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second or further container. The container or containers are placed within a package, and the package can optionally include administration or dosage instructions in the form of a label on the package or in the form of an insert included in the packaging of the kit. A kit can include additional components such as syringes or other means for administering the agents as well as diluents or other means for formulation.

Thus, the kits can comprise: a) a pharmaceutical composition comprising at least one fibric acid or statin derivative compound or composition described herein and at least one lipid altering agent and/or PDE inhibitor and a pharmaceutically acceptable carrier, vehicle or diluent; and b) a container or packaging. The kits may optionally comprise instructions describing a method of using the pharmaceutical compositions in one or more of the methods described herein (e.g., preventing or treating dyslipidemia, hyperlipidemia, hypercholesterolemia, hypertriglyceridemia, sitosterolemia, familial hypercholesterolemia, xanthoma, combined hyperlipidemia, lecithin cholesterol acyltransferase deficiency, tangier disease, abetalipoproteinemia, and fatty liver disease). The pharmaceutical composition comprising the at least one fibric acid or statin derivative compound or composition and at least one lipid altering agent and/or PDE inhibitor contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or more compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter that indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Various patent and/or scientific literature references have been referred to throughout this application. The disclosures of these publications in their entireties are hereby incorporated by reference as if written herein. In view of the above description and the examples below, one of ordinary skill in the art will be able to practice the disclosure as claimed without undue experimentation.

Although the foregoing disclosure has been described and depicted in terms of certain preferred embodiments, other specific embodiments may be effected by those skilled in the art to accomplish the same objectives and without departing from the true spirit of the scope of the present disclosure. Accordingly, the scope of the Applicant's disclosure is to be determined by reference to the attached claims, which are not limited to any of the particular embodiments disclosed herein.

1-149. (canceled)

150. A compound represented by the structure (I) 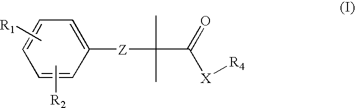
wherein
R¹ is chosen from H and halogen;
R² is chosen from H, halogen, cycloalkyl substituted with from 1 to 3 halogens, COR³, and (CH₂)ₘNHOR³;
R³ is phenyl substituted with from one to three halogen groups;
Z is chosen from O and (CH₂)ₙO;
X is chosen from direct bond, O, NH, and an amino acid residue;
R⁴ is chosen from OH, NO, NO₂, an amino acid residue, a fabric acid residue, a guanidine residue, a tetrazolyl residue, an agmatine residue, an amino-containing compound residue; a lower alkyl group terminating in ONO, (ONO₂)ₚ, or guanidine; a resveratrol residue; and an imidazoline receptor agonist residue; and
m, n, and p are independently chosen from 1 to 3.

151. A compound according to claim 150 represented by the structure (II):

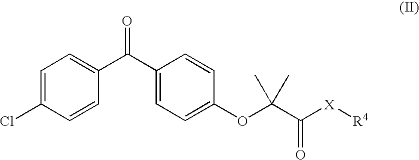

152. The compound according to claim 151, wherein X is O and R⁴ is a lower alkyl terminating in (ONO₂)ₚ, and p=1.

153. The compound according to claim 152, chosen from the following structures:

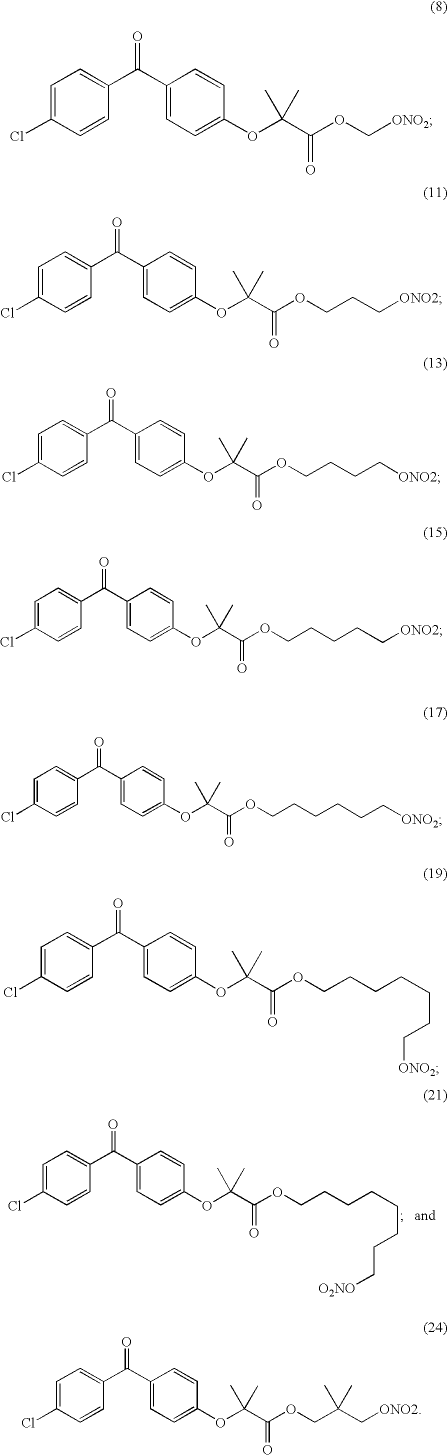

154. The compound according to claim 151, wherein X is NH and R⁴ is OH.

155. A composition for the prevention and/or treatment of lipid related disorders, said composition comprising: (1) a pharmaceutically acceptable carrier and (2) a therapeutically effective amount of a compound represented by the structure (I) 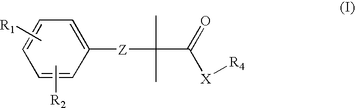
wherein
R¹ is chosen from H and halogen;
R² is chosen from H, halogen, cycloalkyl substituted with from 1 to 3 halogens, COR³, or (CH₂)ₘNHOR³;
R³ is phenyl substituted with from one to three halogen groups;
Z is chosen from O or (CH₂)ₙO;
X is chosen from a direct bond, O, NH, or an amino acid residue;
R⁴ is chosen from OH, NO, NO₂, an amino acid residue, a fabric acid residue, a guanidine residue, a tetrazolyl residue, an agmatine residue, an amino-containing compound residue; a lower alkyl group terminating in ONO, (ONO₂)ₚ, or guanidine; a resveratrol residue; or an imidazoline receptor agonist residue; and
m, n, and p are independently chosen from 1 to 3.

156. The composition according to claim 155 comprising a compound represented by the structure (II)

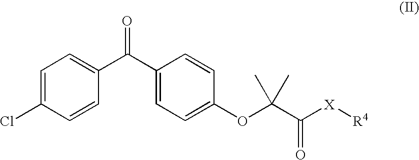

157. The composition according to claim 156, wherein said compound (II) is chosen from the following structures:

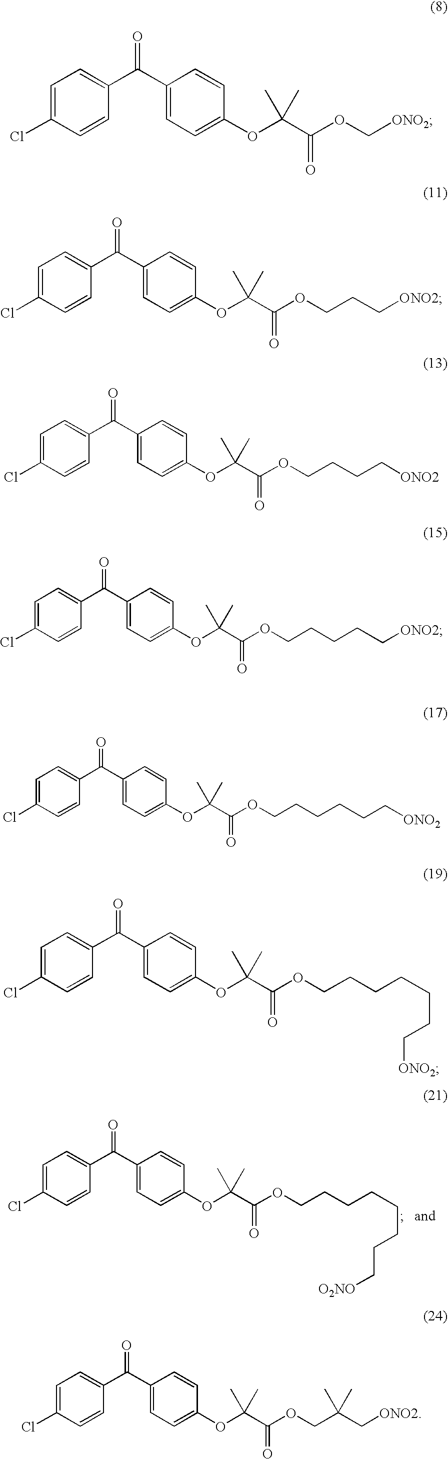

158. The composition according to claim 156, wherein said compound (II) is chosen from the following structures:

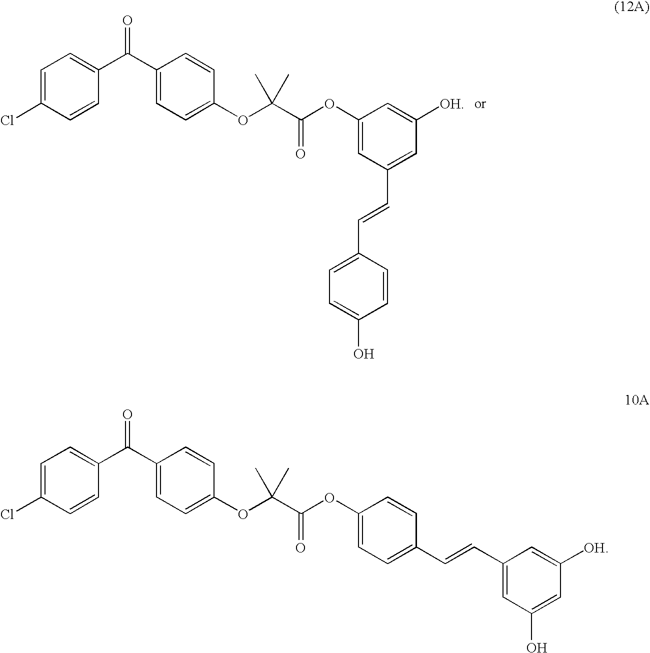

159. The composition according to claim 156, wherein said compound (II) is chosen from the following structures: 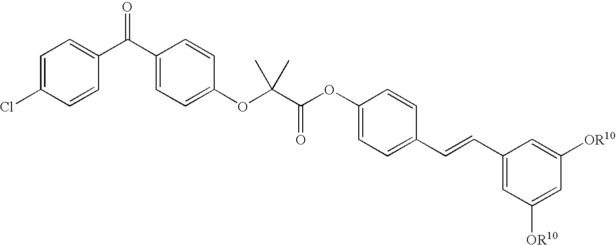
wherein R¹⁰ is C₁-C₆ acyl.

160. The composition according to claim 159, wherein said compound (II) is represented by the following structure:

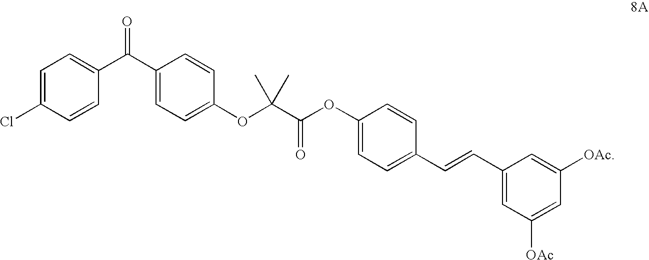

161. The composition according to claim 156, wherein said compound (II) is chosen from the following structures: 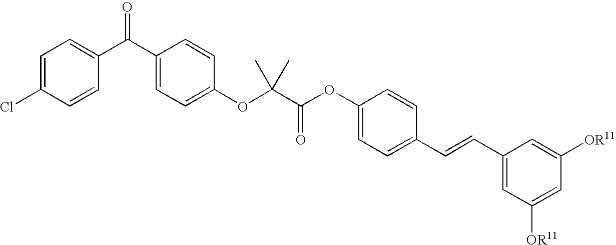
wherein R¹¹ is C₁-C₆ alkyl.

162. The composition according to claim 161, wherein said compound (II) is represented by the following structure:

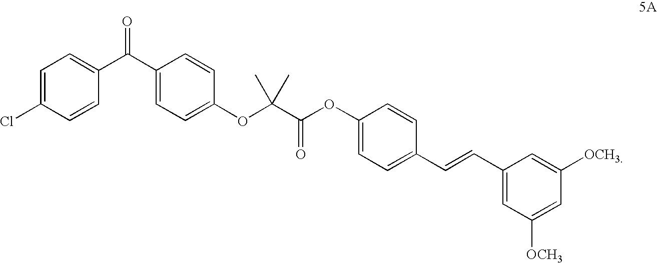

163. The composition according to claim 155 further comprising at least one lipid altering agent.

164. The composition according to claim 163, wherein said lipid altering agent is chosen from among: statins, fibrates, cholesterol-ester-transfer-protein (CETP) inhibitors, squalene synthase inhibitors, microsomal-triglyceride-transfer-protein (MTTP) inhibitors, cholesterol absorption inhibitors, soluble guanylate cyclase modulators, bile acid sequestrants, thyroid receptor agonists, LXR modulators, or antisense inhibitors of apoB-100 or C-reactive protein.

165. The composition according to claim 155 further comprising a therapeutically effective amount of at least one phosphodiesterase inhibitor.

166. A kit for treating a lipid metabolism disorder comprising, in one or more containers, a therapeutically effective amount of the composition according to claim 155, together with a label or packaging insert containing instructions for use.

167. A method for treating or preventing a lipid metabolism disorder, said method comprising: administering to a patient in need thereof a therapeutically effective amount of a composition comprising a compound represented by the structure (I) 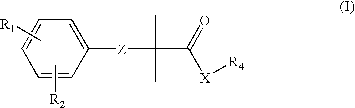
wherein
R¹ is chosen from H and halogen;
R² is chosen from H, halogen, cycloalkyl substituted with from 1 to 3 halogens, COR³, or (CH₂)ₘNHOR³;
R³ is phenyl substituted with from one to three halogen groups;
Z is chosen from O or (CH₂)ₙO;
X is chosen from a direct bond, O, NH, or an amino acid residue;
R⁴ is chosen from OH, NO, NO₂, an amino acid residue, a fabric acid residue, a guanidine residue, a tetrazolyl residue, an agmatine residue, an amino-containing compound residue; a lower alkyl group terminating in ONO, (ONO₂)ₚ, or guanidine; a resveratrol residue; or an imidazoline receptor agonist residue; and
m, n, and p are independently chosen from 1 to 3.

168. The method according to claim 167 wherein said composition comprises a compound represented by the structure (II)

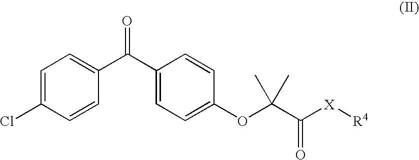

169. The method according to claim 167, wherein said lipid metabolism disorder is chosen from: dyslipidemia, hypercholesterolemia, hyperlipidemia, hypertriglyceridemia, sitosterolemia, or fatty liver disease.

170. The method according to claim 167, wherein said patient is diabetic.

171. The method according to claim 167, wherein said method is effective in said patient for: (i) lowering glycosylated hemoglobin levels (HbA_{1C}); (ii) lowering fasting plasma glucose (FPG) levels: (iii) lowering peak and 2-hour post-prandial glucose (PPG) levels; (iv) improving insulin sensitivity or reducing insulin resistance; (v) increasing insulin secretion; or (vi) reducing the risk of developing diabetes-associated complications.
